SEQUENCE LISTING (47 sequences)

SEQ ID NO: 1 - protein, 695 aa, synthetic construct
MGSASGVDELVRIKQNYARMLVPSGKDPFGLLSILSSIQPETEISDQVVVELHQRYPFDLKKVETYLSSFTEAGTWPDINYDDKKRSGWEPKIHAERILELVKLYNSDQTSYYRSSEVEAVIHKALNYWFTAKPVCLNWWYNQIGIPKTLGTVFILFEKQLTPVEKQNAITVMENAKFGMTGQNKVWLAGNVMMRALLQNDYELVKMARDTIASEIVTGGTEGIKDDWCFHQHGAQQQFGNYGLSFVSGMSFFSGLFSGTSLAFDDKQLSILSTLIDKGYRWVIWKGMMDVNALGRQLFHHAPVHKALSLAFAASELGGGESDECMAVATALLRDNYPAPAVNVLTGHKHFWQSDYTIHRRPSWMASIKMASDRIIGTEMMNGDNMKGYYMADGATYIYKDGKEYLDIFPLWDWRKLPGVTAFEDNAPMPLIKSYQPRNKGTFVGAVSDEKQGMTVMELDRAGVKAHKAWVCTDDFILCLGAGIQADSNLVVTTSIEQCHKNGELLSWENARWNVVSTKQSVKGKEQRYFHNNTGYIVWGNTHEVVAETAERTGSWYDVMQMYHPEETHGEVTAIYLTHGVAPKQGTYQYLILPGMDKENVAAFNFSDIRILRNDATVQAVYSEGNTTCWVAAYQPVQLTVSTDLILNIQTPGIYMIRKNESGRYIINYADPTQQRNVAELELNHKKVRLSLLEG

SEQ ID NO: 2 - protein, 704 aa, synthetic construct
MGSGVDELVRIKQNYARMLVPSGKDPFGLLSILSSIQPETEISDQVVVELHQRYPFDLKKVETYLSSFTEAGTWPDINYDDKKRSGWEPKIHAERILELVKLYNSDQTSYYRSSEVEAVIHKALNYWFTAKPVCLNWWYNQIGIPKTLGTVFILFEKQLTPVEKQNAITVMENAKFGMTGQNKVWLAGNVMMRALLQNDYELVKMARDTIASEIVTGGTEGIKDDWCFHQHGAQQQFGNYGLSFVSGMSFFSGLFSGTSLAFDDKQLSILSTLIDKGYRWVIWKGMMDVNALGRQLFHHAPVHKALSLAFAASELGGGESDECMAVATALLRDNYPAPAVNVLTGHKHFWQSDYTIHRRPSWMASIKMASDRIIGTEMMNGDNMKGYYMADGATYIYKDGKEYLDIFPLWDWRKLPGVTAFEDNAPMPLIKSYQPRNKGTFVGAVSDEKQGMTVMELDRAGVKAHKAWVCTDDFILCLGAGIQADSNLVVTTSIEQCHKNGELLSWENARWNVVSTKQSVKGKEQRYFHNNTGYIVWGNTHEVVAETAERTGSWYDVMQMYHPEETHGEVTAIYLTHGVAPKQGTYQYLILPGMDKENVAAFNFSDIRILRNDATVQAVYSEGNTTCWVAAYQPVQLTVSTDLILNIQTPGIYMIRKNESGRYIINYADPTQQRNVAELELNHKKVRLSLLEGKEKGKTTSIVG

SEQ ID NO: 3 - protein, 706 aa, synthetic construct
MGSASGVDELVRIKQNYARMLVPSGKDPFGLLSILSSIQPETEISDQVVVELHQRYPFDLKKVETYLSSFTEAGTWPDINYDDKKRSGWEPKIHAERILELVKLYNSDQTSYYRSSEVEAVIHKALNYWFTAKPVCLNWWYNQIGIPKTLGTVFILFEKQLTPVEKQNAITVMENAKFGMTGQNKVWLAGNVMMRALLQNDYELVKMARDTIASEIVTGGTEGIKDDWCFHQHGAQQQFGNYGLSFVSGMSFFSGLFSGTSLAFDDKQLSILSTLIDKGYRWVIWKGMMDVNALGRQLFHHAPVHKALSLAFAASELGGGESDECMAVATALLRDNYPAPAVNVLTGHKHFWQSDYTIHRRPSWMASIKMASDRIIGTEMMNGDNMKGYYMADGATYIYKDGKEYLDIFPLWDWRKLPGVTAFEDNAPMPLIKSYQPRNKGTFVGAVSDEKQGMTVMELDRAGVKAHKAWVCTDDFILCLGAGIQADSNLVVTTSIEQCHKNGELLSWENARWNVVSTKQSVKGKEQRYFHNNTGYIVWGNTHEVVAETAERTGSWYDVMQMYHPEETHGEVTAIYLTHGVAPKQGTYQYLILPGMDKENVAAFNFSDIRILRNDATVQAVYSEGNTTCWVAAYQPVQLTVSTDLILNIQTPGIYMIRKNESGRYIINYADPTQQRNVAELELNHKKVRLSLLEGKEKGKTTSIVG

SEQ ID NO: 4 - protein, 693 aa, synthetic construct
MGSGVDELVRIKQNYARMLVPSGKDPFGLLSILSSIQPETEISDQVVVELHQRYPFDLKKVETYLSSFTEAGTWPDINYDDKKRSGWEPKIHAERILELVKLYNSDQTSYYRSSEVEAVIHKALNYWFTAKPVCLNWWYNQIGIPKTLGTVFILFEKQLTPVEKQNAITVMENAKFGMTGQNKVWLAGNVMMRALLQNDYELVKMARDTIASEIVTGGTEGIKDDWCFHQHGAQQQFGNYGLSFVSGMSFFSGLFSGTSLAFDDKQLSILSTLIDKGYRWVIWKGMMDVNALGRQLFHHAPVHKALSLAFAASELGGGESDECMAVATALLRDNYPAPAVNVLTGHKHFWQSDYTIHRRPSWMASIKMASDRIIGTEMMNGDNMKGYYMADGATYIYKDGKEYLDIFPLWDWRKLPGVTAFEDNAPMPLIKSYQPRNKGTFVGAVSDEKQGMTVMELDRAGVKAHKAWVCTDDFILCLGAGIQADSNLVVTTSIEQCHKNGELLSWENARWNVVSTKQSVKGKEQRYFHNNTGYIVWGNTHEVVAETAERTGSWYDVMQMYHPEETHGEVTAIYLTHGVAPKQGTYQYLILPGMDKENVAAFNFSDIRILRNDATVQAVYSEGNTTCWVAAYQPVQLTVSTDLILNIQTPGIYMIRKNESGRYIINYADPTQQRNVAELELNHKKVRLSLLEG

SEQ ID NO: 5 - protein, 723 aa, Bacteroides intestinalis
MRLLRNLIIISVLLVSVTVQASGVDELVRIKQNYARMLVPSGKDPFGLLSILSSIQPETEISDQVVVELHQRYPFDLKKVETYLSSFTEAGTWPDINYDDKKRSGWEPKIHAERILELVKLYNSDQTSYYRSSEVEAVIHKALNYWFTAKPVCLNWWYNQIGIPKTLGTVFILFEKQLTPVEKQNAITVMENAKFGMTGQNKVWLAGNVMMRALLQNDYELVKMARDTIASEIVTGGTEGIKDDWCFHQHGAQQQFGNYGLSFVSGMSFFSGLFSGTSLAFDDKQLSILSTLIDKGYRWVIWKGMMDVNALGRQLFHHAPVHKALSLAFAASELGGGESDECMAVATALLRDNYPAPAVNVLTGHKHFWQSDYTIHRRPSWMASIKMASDRIIGTEMMNGDNMKGYYMADGATYIYKDGKEYLDIFPLWDWRKLPGVTAFEDNAPMPLIKSYQPRNKGTFVGAVSDEKQGMTVMELDRAGVKAHKAWVCTDDFILCLGAGIQADSNLVVTTSIEQCHKNGELLSWENARWNVVSTKQSVKGKEQRYFHNNTGYIVWGNTHEVVAETAERTGSWYDVMQMYHPEETHGEVTAIYLTHGVAPKQGTYQYLILPGMDKENVAAFNFSDIRILRNDATVQAVYSEGNTTCWVAAYQPVQLTVSTDLILNIQTPGIYMIRKNESGRYIINYADPTQQRNVAELELNHKKVRLSLLEGKEKGKTTSIVG

SEQ ID NO: 6 - protein, 301 aa, synthetic construct
KIVKQGTDEMVVLRGVNVPSMDWGMAEHLFESMTMVYDSWGANLIRLPINPKYWKNGSVWDEKNLTKEQYQKYIDDMVKAAQARGKYIILDCHRYVMPQQDDLDMWKELAVKYGNNSAVLFGLLNEPHDIKPVGVEKPTTVEQWDVWYNGGQIIVGGEEVTAIGHQQLLNEIRKQGANNICIAGGLNWAFDISGFADGYNERPNGYRLIDTAEGHGVMYDSHAYPVKGAKTAWDTIIGPVRRVAPVIIGEWGWDSSDKNISGGDCTSDIWMNQIMNWMDDTDNQYDGIPVNWTAWNLHMSS

SEQ ID NO: 7 - protein, 884 aa, synthetic construct
MEEAAADAQNAEINYNRSVPLEVKGNKIVKQGTDEMVVLRGVNVPSMDWGMAEHLFESMTMVYDSWGANLIRLPINPKYWKNGSVWDEKNLTKEQYQKYIDDMVKAAQARGKYIILDCHRYVMPQQDDLDMWKELAVKYGNNSAVLFGLLNEPHDIKPVGVEKPTTVEQWDVWYNGGQIIVGGEEVTAIGHQQLLNEIRKQGANNICIAGGLNWAFDISGFADGYNERPNGYRLIDTAEGHGVMYDSHAYPVKGAKTAWDTIIGPVRRVAPVIIGEWGWDSSDKNISGGDCTSDIWMNQIMNWMDDTDNQYDGIPVNWTAWNLHMSSSPKMLYSWDYKTTAYNGTHIKNRLLSYNTAPEKLDGVYSTDFSTDDVFRSYTAPSGKASIKYSDESGNVAITPAAANWYATLNFPFDWDLNGIQTITMDISAATAGSVNIGLYGSDMEVWTKAVDVNTEVQTVTIGINELVKQGNPQTDGKLDAALSGIYFGAATADTGSITIDNVKIVKLATPVYTANTYPHKDMGEESYIDIDTTGFKKQTTAWNSKFTGTTMQITDANVLNINGETTKTKCVTYTRDATDTEGCRAKFDLNTVPSMDAKYFTIDIKGNGIAQKLTVSLSGLAYITVNMAEGDTDWHQYIYSLEGNVEYPEDITYVQISADTRTTAEFYIDNIGFSNTKSERLIPYPEKTFVYDFATYNKNTTKYEAAISTESGSEGDTIVATKEEGGLGFDSKALEVKYSRNGNTPSKAKVVYSPNDFFKGNVNDDERTANRATLKADMEYMTDFVFYGKSTSGKNEKINVGVIDTASAMTTYTDTKEFTLTTEWKQFRVPFDEFKILDGGSNLDCARVRGFIFSSAENSGEGSFMIDNITHTSIKGDIEWGLP

SEQ ID NO: 8 - protein, 2149 aa, unidentified
MERVIFMKKFLSLVTAIVMTVSLCIMPVYAQTYEEAAADAQNAEINYNRSVPLEVKGNKIVKQGTDEMVVLRGVNVPSMDWGMAEHLFESMTMVYDSWGANLIRLPINPKYWKNGSVWDEKNLTKEQYQKYIDDMVKAAQARGKYIILDCHRYVMPQQDDLDMWKELAVKYGNNSAVLFGLLNEPHDIKPVGVEKPTTVEQWDVWYNGGQIIVGGEEVTAIGHQQLLNEIRKQGANNICIAGGLNWAFDISGFADGYNERPNGYRLIDTAEGHGVMYDSHAYPVKGAKTAWDTIIGPVRRVAPVIIGEWGWDSSDKNISGGDCTSDIWMNQIMNWMDDTDNQYDGIPVNWTAWNLHMSSSPKMLYSWDYKTTAYNGTHIKNRLLSYNTAPEKLDGVYSTDFSTDDVFRSYTAPSGKASIKYSDESGNVAITPAAANWYATLNFPFDWDLNGIQTITMDISAATAGSVNIGLYGSDMEVWTKAVDVNTEVQTVTIGINELVKQGNPQTDGKLDAALSGIYFGAATADTGSITIDNVKIVKLATPVYTANTYPHKDMGEESYIDIDTTGFKKQTTAWNSKFTGTTMQITDANVLNINGETTKTKCVTYTRDATDTEGCRAKFDLNTVPSMDAKYFTIDIKGNGIAQKLTVSLSGLAYITVNMAEGDTDWHQYIYSLEGNVEYPEDITYVQISADTRTTAEFYIDNIGFSNTKPERLIPYPEKTFVYDFATYNKNTTKYEAAISTESGSEGDTIVATKEEGGLGFDSKALEVKYSRNGNTPSKAKVVYSPNDFFKGNVNDDERTANRATLKADMEYMTDFVFYGKSTSGKNEKINVGVIDTASAMTTYTDTKEFTLTTEWKQFRVPFDEFKILDGGSNLDCARVRGFIFSSAENSGEGSFMIDNITHTSIKGDIEWGLPTPTPEPTPTPLPDPVTVTTAEQLAAITSTEGNIILGADIDLGTTGFTTKSVTHLDLNGHTLTSSGPFVVDPRHEITIVDTGSTKGAIINTGTTQTSYGIRGTTEAATINIDGAEIDAGGQAILINVAGRKCNIKDAVINGGSYAINVGTNGGEINIDNALINNKADYKGYALYLQGGIAIIDDGTFGYNGTTNTLLVARSSELTINGGTFTNPNSGRGAIVTDKQFVGTVTINGGVFENTNAGGYSILDSNEGYQSIDAETSEIIASPVININDGTFKSAIGKTKSTNSSATEISIKGGQFAADPTVLYPNCIDTDIYSITKVAEGKYVVTKKGVEPTPEPTPEPVAKIVSSIEEINTLTASDDYVKLGADIDLGTSSIKTKCAMRLDLNGHTLSGGGSTVIEAMYNLTVVDTGTTKGTIKNVNTSTSYGIKFAVKDAVLTIDGAKVEAMSQAIMLSGTGSILHLKDSVINGNSYAVNLSNGIINIENTVINDDSEYKGYALSVANGTAVINSGIFNYNGNMSSITFSGSSEITINGGTFKNSVSKRGAINTVKGFSGTLTINGGTFENTAENNGYSILDGDEATTETVPVINITGGTFKSTIGATKPANTTTVITISGGTYSFDPTSYVTDTETYRVIDNGDGTYKVAPNSQVYSVTLNACGGSEVMVEDFKEENIPDNGIELPIPTKAGYKFDGWYTEENNGSQVNGITKDNLSDIFRNEATVTLYAHWTLLNYTITYEGLNDATNTNPSNYTVETEAITLAAPGTRKGYTFGGWYTDVEYQNKIEIIEQGTTGNKILYAKWDEIASGSITASFVSTGTIPSDIVQGTINVTEKAYENDEVSFMVTLPKGYTLENVLCTADGENLNTITEENGSYTFIMPGKNVTITVNVRPIQYTINLDLQEGTGTTTTIYGSVENLPVLPNDNPKKQGYNFKGWFDAPTKGTVITMDNLNTASNMLALFGNNTELTIYAQYTEVGNFVVIYSAVGADEETIPTDNTQYNIAETSIIKIPNQEPKKLGYTFEGWKTGTDDTVYKYGTQNDTYTVPNDINGAITFIAQWSINEYEITYELNGGINAENAPVSYTIETDTITLPVPTKDGYNFEGWYTDAAFENAVAAIAKGSVGDMVLYAKWSEKDMAVYKINNYEKGNVSVRKRTDTDDSSSVVIVAFYKTLNNNSVLIKTSIAEIGAIEKGDDISKTVEEPEDYSYAKVFMWNDLNGMMPRCNSPKMDK

SEQ ID NO: 9 - protein, 727 aa, unidentified
MILLIHIKMGGMIMTDFNILRKRYSDVLCGRGYNGKKTADCILQSDERTEQRLVQLGGRIEKAITSNEPGVINATLKGILDISISFSQNNSQFYHNKNIKNEIFNALNTLEKVYNDTTVPKGNWWYWEIGIPLSINSIFTLMYDYTDKSQLKRYMAAEKHFNDRIKLTGANRIWESVIFAVRGILLSDNDSIKNAISGIQDVMVITDSGDGFYKDGSFIQHDNIPYNCGYGRSLIQELAPMLYIFKDTEFENKNTDIINTWIEKSYLPFIYNGRAMDMVRGREISRYYEQSDLACTHILSAMLILSEMPEFNELKGTIKTQITDNFFEYASVFTAELAEHLQEDNNIKPKEIKPYFMAFNSMDRVVKHGNGYTIGLAMHSERTAAYESINDENQNAHHTSDGMMYIYKKNEPKSDFFWQTIDLQRLPGTTVLRGSTVKPNINAAGDFTGGCGIGENGVCTMKLISNENSLKANKSWFFFDKEVVCLGSCINSEEESEVETIIENRLVTDNSRFTVHGNEESEGYIIKGAYLDGSHDVGYCFPEEQEVNIFREIRSGDWNNMSIKSDGKSYKGRYLTMWIKHGRKVKDVSYEYIVIPKCHEEEINDYYRKSGIRIIENSDSIQCVKKNGTTGVVFLKDKTHSAGGISCDRRCIVMTTQTCGTLELSISDITQKQDKIYIELDYSAQEIISKSERINIIQLVPYVCMEIDTCAARGEEQHIKFGGVKNV

SEQ ID NO: 10 - protein, 815 aa, unidentified
MENLLVRRTNMKYGYFDDLNKEYVIETPRTPLPWINYLGTNGFFSLISNTSGGYCFYKDAKHRRILRYRYNNIPADNGGRYFYINDNGDCWTPSYMPMKKELDFYECRHGMGYTKITGERNGVRVEQTAFVPVDDNCEIHRIKVTNTSGEAKNINLFSFVEFCLWNAQDDMLNYQRNLNTGEVEIDGSAIYHKTEYRERRNHYAFYSVNTEISGFDTDRDTFLGAFNGLDTPDRVINGKSGNSVASGWYPIASHQIDVSLDAGESREYIFVLGYIENEKDEKFESLNVINKTKAKEMIARYESSAQCDAELDKLKLYWDNLLSVFTLESNDEKLNRMVNIWNPYQCMVTFNMSRSASYYESGIGRGMGFRDSNQDLLGFVHQIPERARERIIDIASTQFEDGSAYHQYQPLTKQGNNEIGGGFNDDPLWLILGTVAYIKETGDYGILDEQVPFDCDKNNTATLLEHLNRSFGHVTNNLGPHGLPLIGRADWNDCLNLNCFSEIPDESFQTTGDDDGRVAESVLIAGMFVYIGREFARLYKTLNNDEMYKYISDEVEKMTEAVLEYGYDGEWFIRAYDANGNKVGSDECDEGKIFIESNGFCVMAGIGKEDGRAQKALDSVKKYLECEYGIVLNYPPYSGYRLELGEISSYPPGYKENAGEFCHNNPWVIIGETVMGNGERAFELYKKIAPAYLEEISEIHKTEPYVYSQMIAGRDAVRAGEAKNSWLTGTAAWNYYTVSQYLLGIRPDFDGLVIEPCISKDISEFKVTRKFRGKTYNILVKNTGEGTVKITADNGTVNGTTVSSDAEICNVEVVM

SEQ ID NO: 11 - protein, 928 aa, unidentified
MILIYNSDIMYNKYIKPKFIIWYKKEFQMSKNVHIISHSHWDREWYLPFEQHRMRLVELIDKCMEVFEKDDSFKSFFLDGQTIVLDDYLEIRPENKEKLIKYTKEGKFIIGPWYILQDEFYTSGEANIRNLLVGMKEAEKYGAMCKMGYFPDAFGNAGQMPQLLKQAGMDTVTFGRGVRPVGFDNEVQENGNYESPYSEMMWESPDGTKIFGILFANWYNNGNEVPTDKKIAKEYWDDRLKKVATFASTDEYLLMNGCDHQPVQADLGKAIEVASELYPDINFKHSNFPEYIKAIKEKVPNDLAVVKGELTSQDTDGWSTLMNCASSHIYLKQMNRKCESALENGAEPVRVLSSVLGQNYPSDELEYSWKKLMQNHPHDSICCCSVDEVQDEMATRFNKSKQVADYLVSEGKRYIADKINTKEYEKYKNALPFVVFNTAGRERTSVVSVEIDVTRKSGWLKKCAYDLDEINVPNYKLIDSDGNSIPFKIEDLGVKFGYDLPKDKFRQPYMARRVRVTFEAENISAVGYKTYALVEGDTEKVTDTLVSSENCMENDAIRVEINKNGSLNVTDKASGRTYKGVAYYEETGDLGNEYMYKMPEGSKAITTQDTVAKIELAEDEPYRAMYKITNTITVPKSGDDNFEDEKSHMVFFKERVGGRSNDTVEMKIETFVSLDKNGKGVKIKTRFDNEVKDHRVRIMVPTGINSDVHKADSVFEVVTRNNRHNAGWNNPSACEHEQGFVSIDDGEKGIAVANIGLYEYEMLPDLDNTIAVTILRAVGEMGDWGVFPTPKAQCLGISETEIEIVPFKGDLISSGAYEECYQFRTDIITADTDCHDGVMPLDYSMINWQGNGLILTGIKQKGNGEDIIIRWVNVSDKTTTLTIQKSDVIDNLYISNIIEKKIKKIDSNNNYFNIEVKPYEIMTVGIAK

SEQ ID NO: 12 - protein, 950 aa, unidentified
MERKNIKCHFISNTHWDREWKFSAQRTRHMLVTAIDMLLDIFEKEPDYKHFHLDSQTLPIQDYLEINPEKKEILKKYISEGKLAVGPWFCLPDEFCVGGESLIRNLLLGHKIANEFGKVSKTGYSPFGWGQISQMPQLYHGFGIDFASFYRGLNTYMAPKSEFYWEGADGTTIYASRLGQRPRYNMWYIMQRPVFYGKRDGDNRRVSWGAGDGIFRFADPARCEYEYQYSHRKYEYHDEYIAEKTEQALSEQDDEWTTPNRFWSNGHDSSIPDMRESRLIKDANAVYEGVDVFHSTVYDFEQSVIRDFDKNSPVLKGEMRYPFTKGSVSALFGWVLSARIKVKQENFETERLLTSYAEPMAVFASVCGAVYPQAFINKAYNYMLQNHGHDSIGACGRDVVYKDVEYRFRQSREIATCVLERALMDLSGDIDFAGWDKNDMALVMFNPAPFKRSLTVPCELEIPLEWECDSFEIVDAEGNVCPHQNISSINPMYQIVQDLADAVDVLPVSRHTIRIFVKDIPSMGYKTLKVVPKYHTRATTPVNMLCGINTMENEYLKVKINSNGTLKVTEKETGREYDNIGYFKDTGENGSPWEHKTPELDEEYTTVNERAIVSLVYSGELETKYRIVLNWAIPENIVDGGKKRSSRLAPYRIETLVTLRKGARWVEFETKINNNVPNHYLQAAFPTDVDAEFVYAQGQFDVVKRPIAKPDYSKYDEIPMTEQPMNSFVDICNENEGAAILNTGLKAYESDDDYNHTVYLSLLRCFELRIYVTPEEQNYSRIENGSQSFGEHTFRYAFMPHKGDWEDAQVWKAAEDFNMEILIGQTAPTEHGKNPLEKSFIELENENLHISAVKRSEDGLGCVVRLFNPSSETVKNRIRFNGGIAEISDKQSPIERQVHSFELPCTENRKWASVKKVTLEELSETELSVDTNGWCDVEVTPKQIYTLKYE

SEQ ID NO: 13 - protein, 367 aa, unidentified
VNIDKAITYAESIVRKSLNYFYDCFPTEQSENLVFKKFENVSWTTGFYEGILWLMYELTGDKAFYNSAKHHSEMFHKRLVDRVELEHHDMGFLFTLSSVADYRITGDEQAKQDGIEAAEWLLKRYQPKGKFIQAWDAMDDSQSYRFIVDCMLNIPLLFWASEVTGYKKYYDAAYNHMQTSIANIIRPDASSYHTFFFDPVTNKPLRGETHQGFSDDSSWARGQSWAVYGLALCYHYTKEKSILPLFERVTHYFIDHLPEDSVPYWDLIFSDGSDEPRDTSAAVVAVCGILEMEKYYHNQEFLDAAEKMMTSLSEKYTTVDYPQSNGIIKDGMYSRKHGHEPECTSWGDYFYLEALMRMKKSDWKIYW

SEQ ID NO: 14 - protein, 1604 aa, unidentified
MKKIISLMLAVTMICASIGLTAFAATTTTVEAEADGVSAYTLPSSDKSNSKILKNTVSSKESVTYYIQANNTPRATMFKLAQVNTGDKINVDINFTYLDTATMELEYCLFVSDSEITLTSHSQDLVKEELEKHTDESNIKNWSTNKSNMKYSLPNGITASKDGFVYLYIGCGDLSEDKTQVTKKIQWSIDSFDVNIDSDGGGETEPDTTPTPTTTINPDVTPTPTPTASPTPTPTLEPELTLNAVYSSNMVLQRKEPITITGTGKSGNTVSVNFNGADEQTTIEHGLWEITLPAMEAVKSATMTVSSGDNMITLDNVAVGDVIFCTGQSNMFNRLETFPTLMNEELSEAYEDVRYMNSFDEISEWKVATMENSKQFSALGFLIGKRMIKKDSDVPIGLISSSLGGSSIMQWIPTYSVNWDSQAKRMMAGASSKGGLYTQRLLPLKNLKASAVVWYQGEANTTFESGTVYEQALTSLINNWRKTFNDEDLPFVVIQLPTANFAKIYSTIRIGTGVRAGQWNVSQRMDNVKTVVSNDTGTTNNVHPNDKGPIADRAVAYIEDFINNTQSNVESPSFDYMERSGDKLILHFKNTYGSLSTDDGGVPLGFELKDDDGIYKDVTPTINGDTIEIDVTDITNPQVKYAWSDTPGIAKDLVEAQTDTPAVINTFNAAGRPIAPFMTDLTEKYASKAVNKELSTTEFYNYAPYISKVEQSGDDIVISAYDTDGVVSKVEVYIDEGEIKAGDAKQRDDGKWVFTPDVTSGVHSVYAIATDNDNINSLTCVDYTTYNIIRPTRYDYVKGYTESPSSVEYNNGDDMLAKATNDVNGTTTTVTSAIPTGETTKSLKLSATGNKATANATIPISKADNPQKTLTIEYDTMFESADDAIGASRGMYAKTKEGNELWLTYFTASSLRTAITNTGGNWCYEQAMSIKNNQWHHIKLELHPNTGIFSIWLDGTMLQDNVSFVKEGSSFDTCKGAFDTLKEGITDLRFYHTASNNIENATYIDNVKVTEVSYSEEEIIPPAKIQEATPQISIDYINETLTGFESQEPYTIKVGEGNAKDITLGEGVTTISLDDEKIGYAGKLLSIEIVKKARNTETYTDSDVQQLTVKARPKAPTTVQGVNATEIGGKGKLTGMNGMQYKLKRTDEWSSTQLVDTVEVDAGEYNVRKAATDTDFASEKTTITVETFIAEKEMTPEIAIDYTTEELINFVEDGTYTINGLDVTLTDNKLSLANYITNEQITLSIVKKGNNVTTVASEAQTLIVKARPAAPTKSEIIVTQPSVIGGKGTIAGIADTMEYSTNNGINWTTGDGDDIGDIEPGTTYKIRYKAVSADEEAERQFKSAEYSVTIIAYDAMPETQPTISINYVNEKLTGFTEGCDYIIKIDDGVATDKDNVTEDIDIDNTYFGHTLKIVKKDDGIKTSNSEAFELSIPKRSSAPNVAAVEEQTYQGNDGKITGVDTTMEYKSLSEPTFTWMQCVGTEITNLAPGSYIVRVAAVADESFASEVMSVTINAAAKDEPTEPTVNITYDDKNGNVNAIFTNITEEGMVYVAEYNENGTLLSIKSDEISDSVIIPFTCVNKSKVKVFIWKNDMKPLFNKVFTLN

SEQ ID NO: 15 - protein, 266 aa, unidentified
MFNKKFNLLKEATEYGFMPYMETYILDGKKRPIVVIFPGGGYGMVSEREAERIAMAYNAAGFHAAVVYYCVEPHTHPLPIQNAANAVAMLRENAEKWNIDTDKVIVCGFSAGGHLAASLSALWNDSEIFSEREIELAMHKPNAQILSYPVITSGEFAHKDSFKNLTGTDDESNHLWSSLSLERRITDIIPPTFLWHTYEDICVPVENTLMYAAGLRRVGVPFELHIFEKGEHGLSRVSDELIWSKRKFEREYPWLSLSVDWLNQLF

SEQ ID NO: 16 - protein, 532 aa, Bacteroides intestinalis
MKKRHIIGSFLLGLLLTVNTGCEDFLDQKDTSGINENSLFLKPEDGYSLVTGVYSTFHFSVDYMLKGIWFTANFPTQDFHNDGSDTFWNTYEVPTDFDALNTFWVGNYIGISRANAAIPILQRMKDNGVLSEKEANTLIGECYFLRGVFYYYLAVDFGGVPLELETVKDEGLHPRNSQDEVFASVVSDMNIAAGLLPWKAEQGSADRGRATREAALAYQGDALMWLKQYKEAVEVFNQLDSKCQLEENFLNIHEIANRNGKESIFEVQFTEYGSMNWGAFGVNNHWISSFGMPVAISGFAYAYADKKMYDSFENGDLRRHATVIGPGDEHPSPLIDLQDYPKLKDFATKGNGNIPASFYQDEEGNVLNTCGTVENPWLDGTRSGYYGVKYWRNPEVCGTRGAGWFMSPDNIMMMRYAQVLLSKAECLYRLNDSNGAMAIVQKVRDRAFGKLQNSAVEVPAPANTDVLKVIMDEYRHELTGETSLWFLLRRTGEHANYIKEKYGITIPTGKDLMPIPQTQIGLNQNLKQNPGY

SEQ ID NO: 17 - protein, 1047 aa, Bacteroides intestinalis
MKTKFIATFFLLICGSVMFAQTRTVKGKVVDKANEPLIGVAVNIKNTSQGSITDFEGNYSIQVNTENAVLVFSYIGYDKQEIKVGARNVIDVVMHEASIALDQVVVVGYGTSKRGDVTGSISSIDAAEIKKVPVVNVGQALQGRMSGVQVTNNDGTPGAGVQVLIRGVGSFGDNSPLYVVDGYPGASISNLNPSDIQSIDVLKDASAAAIYGNRAANGVVIITTKRGNADKMQLSVDATVSVQFKPSTFDVLNAQDFASLATEISKKENAPVLDAWANPSGLRTIDWQDLMYRAGLKQNYNLSLRGGSEKVQTSISLGLTNQEGVVRFSDYKRYNIALTQDYKPLKWLKSSTSLRYAYTDNKTVFGSGQGGVGRLAKLIPTMTGNPLTDEVENANGVFGFYDKNANAVRDNENVYARSKSNDQKNISHNLIANTSLEINPFKGLVFKTNFGISYGASSGYDFNPYDDRVPTTRLATYRQYASNSFEYLWENTLNYSNTFGKHSIDVLGGVSIQENTARNMSVYGEGLSSDGLRNLGSLQTMRDISGNQQTWSLASQFARLTYKFAERYILTGTVRRDGSSRFMRGNRWGVFPSVSAAWRIKEESFLKDVDFISNLKLRASYGEAGNQNIGLFQYQSSYTTGKRSSNYGYVFGQDKTYIDGMVQAFLPNPNLKWETSKQTDIGIDLGFFNNKLMLTADYYIKKSSDFLLEIQMPAQTGFTKATRNVGSVKNNGFEFSVDYRDNSHDFKYGVNVNLTTVKNKIERLSPGKDAVANLQSLGFPTTGNTSWAVFSMSKVGGSIGEFYGFQTDGIIQNQAEIDALNANAHRLNQDDNVWYIASGTAPGDRKFIDQNGDGVITDADRVSLGSPLPKFYGGINLSGEYKGFDFNLFFNYSVGNKILNFVKRNLISMGGEGSIGLQNVGKEFYDNRWTETNPTNKYPRAVWSDVSGNSRVSDAFVEDGSYLRLKNIEVGYTLPANILKKASISKLRIFASVQNLFTITGYSGMDPEIGQSMSSSTGVAGGVTASGVDVGIYPYSRFFTMGFNLEF

SEQ ID NO: 18 - protein, 783 aa, Bacteroides intestinalis
MKTFILSFLIYAGCSLPLTAQQIKPAIPSDPEIEAKINKLLQKLTLEEKIGQMCEITIDVITDFSDKENGFRLSESMLDTVIGKYKVGSILNTPFSIAQEKEVWADLITRIQKKSMEEIGIPCIYGVDQIHGTTYTRGGTFFPQSINMAAAFNRQLTRRGAEISAYETKACCIPWNYAPVMDLGRDPRWPRMWESYGEDCYVNAEMGVQAVKGLQGENPNHIGENNVAACIKHFMGYGVPVSGKDRTPSSISRTDLREKHFAPFLASIQAGALSLMVNSGVDNGVPFHANKELLTGWLKEELNWDGMIVTDWADINNLCLRDHIAETKKEAIQIAINAGIDMSMVPYEVSFCTYLKELVEEGKVSMARIDDAVSRVLRLKYRLGLFDNPYWDIRKYDQFASPEFASVALQAAEESEVLLKNEDDILPLAKGKKILLTGPNANSMRCLNGGWSYSWQGDKADECAQAYNTIYEAFCNEYGKESVIYEPGVTYKTSADALWWEENTPRIAQAVSAAEKADVIIACIGENSYCETPGNLTDLNLSTNQKDLVKALAATGKPIILVLNEGRPRIIHDIVPLAKAVVHIMLLGNYGADALVNLVSGKANFSGKLPFTYPHLINSLATYDYKPCENMGQMGGNYNYDAVMDVQWPFGFGLSYTTYSYSNLKVNRTSFDADNELVFTVDVTNTGKMAGKESVLLYSRDLVASITPDNIRLRNFEKVDLQPGETKTVTMKLKGSDLAFVGADGKWRLEKGAFRMTCGTQKLEVHCTTTKIWQTPNISKSGI

SEQ ID NO: 19 - protein, 564 aa, Bacteroides intestinalis
MKNTVLPLILFLCMLCLGSHLYAGHSMHPLNQISYVKKKIKEQQEPYFTAYRQLMHYADSIQEVSQNALVDFAVPGFYDKPEEHRANSLALQRDAFAAYCSALAYQLSGEERYGQKACYFLNAWSSTNKKYSEHDGVLVMSYSGSALLMAAELMMDTPIWNPQDKDAFKTWVSQVYQKAVNEIRVHKNNWADWGRFGSLLAASLLDDKEEVARNVQLIKSDLFVKIAEDGHMPEEVVRGNNGIWYTYFSLAPMTAACWLVYNLTGENLFVWEHNDASLKKALDYMFYFHQHPSEWKWDTRPNLGAHETWPDNLLEAMAGIYNDASYLQYVESSRPHIYPLHHFAWSFPTLMPVSLKGYDLTDNNTWANYNRYEVANKTVKKPVAIFMGNSITEGWNRSHPDFFTQNGYVGRGISGQVTAQMLARFRADVLDLKPQVVCILAGTNDIAQNCMYMSVENIAGNIFSMAELAKANGIKVVICSVLPATRYSWRPTVQNPAGQIIQLNKLLQKYAQKNKIPYVDFHSMMKDEQNGLPQKYSKDGVHPTKEGFSMMEPIIKEAIDKLLK

SEQ ID NO: 20 - protein, 345 aa, Bacteroides intestinalis
MKNIYYILILCCLCLFSCDSHPDTKSSLPFGVNLAGAEFFHKKMDGVGQFGIDYHYPTTREFDYWKSKGLTLIRLPFKWERIQRELYGELNREEIDYIKYLLDEAGARDMKILIDMHNYGRRKDNGKDRIIGDSVSIDHFASVWKQIAGELKEHSALYGYGLINEPHDMLDSVPWFKIAQAAIEESRKVDLKTAIVVGGNHWSSAARWQEISDDLKHLHDPSDNLIFEGHCYFDEDGSGIYRRSYDEEKAYPTIGIDRTRPFVEWLKTNNLRGFIGEYGVPGDDERWLVCLDNFLDYLSKENINGTYWAAGAQWNKYILSIHPDDNYQTDKIQLGVLTKYLETKN

SEQ ID NO: 21 - protein, 400 aa, Bacteroides intestinalis
MRKQLSLLLVSISLGWVGCAPDKQADTIHLDRQLEYCDAQIRRTLSEADQDSCLMPRSMEANQTNWNMSNIYDWTSGFWPGILWYDYEATGDEEIKAQAIRYTECLLPLVTPAHGADHDIGFQIFCSFGNAYRITGNEEYKTVILKGAQKLAKLYNPKVGTILSWPGMVKRMGWPHNTIMDNMMNLEILFWAARNGGGQELYDIAVKHAQTTMKYSFREDGGNYHVAVYDTIDGHFIKGVTNQGYGDSSLWARGQAWAIYGYMMVYRETQDKTFLRFAEKVTELYLENLPEDYIPYWDFDAPDMIKQPKDASAAAITASALIELSELEDTPSLASRYLNAATRMLGELSSERYQCRDIKPAFLMHSTGNQPGGYEIDASINYADYYYLQALLKYKKAMGL

SEQ ID NO: 22 - protein, 704 aa, Bacteroides intestinalis
MKTRTLGICLFLLMNVSFIKGQSLADKVDMWMGTYGAGHCVVGPQLPHGSVNPSPQTAYGGHAGYVPDQPIRGFGQLHVSGIGWGRYGQIFLSPQVGFNPGETDHDSPKQGEEATPYYYKVMLSRYDIQVEISPTHHCVAYRFTFPETDQGNILLDIAHNIPQHIVPEVKGLFHGGEINYNPEQQTLTGWGEYSGGFGSTDAYKVYFAMKTDTPLKEVKITDQGDKALYACLALNKNPGVVHLNVGISLKSIENASLFLSEEIADNSFNTVKENAKAIWDNTLSSIKIKSENEAEERLFYTTLYHSFVMPRDRTGDNPHWDSESAHMDDHYCVWDTWRTKYPLMVLLRESYVAQTINSFIDRFAHNGVCNPTFTSSLDWTSKQGGDDVDNIIADAIVKNVKGFDYEKAYALMKWNAYHARSKDYLRLGWEPETGGIMSCSAGIEYAYNDFCTSEIAGIMHDENTQKELYERSGNWSQLFNPLQESHTYKGFIVPRKANGEWVAIDPAKAYGSWVEYFYEGNSWTYTLFVPHQFDRLIEYCGGKANMIKRLSYGFENNLISLNNEPGFLSPFIFTHCGRPDLTARYVSQIRKDNFSLLKGYSDNEDSGAMGSWYIFTSIGLFPNAGQDFYYLLPPAFTDVELTMENGKKISIKVLKDTPDACYIKSVSINGKVLDKGWIYHREIAEGATLVYELTNKENAWHINE

SEQ ID NO: 23 - protein, 314 aa, unidentified
MKYYIGIDLGGTNIAAGIVDKTGKIIAKDSVPTLNTRPIEAIMLDMTKLCKTLLDKSQMDINKIEAVGIGCPGTVDNKNGIISYSNNIPMKNVPMRKFMEKQLNISVNLENDANAAALGEYTANGHNASSYILITLGTGIGGGAVINSKIYRGFNGVGIEPGHMTLINGGERCTCGKHGCWETYGSVTALINQTKLKMTDNPDSLMHKISGKFGEVNGRVAFEAAKAGDKAGLEVVEKYTEYVADGITSVINIFEPEILVIGGGISKEGEYLLNPIRKFVEINEFNKYRPKTKIEIASLNNDAGIIGAALSANR

SEQ ID NO: 24 - protein, 738 aa, unidentified
MKKLVSLIIAMSIFFSINCAIFATNVSYMADFESADAKFGNSTTYSGTKNTAGDYSDFVKPEWVADGGKENSTGLRITYKAATWYAGEVFFPIPVAWQNGADAEYLNFDYNGKGIVNISLSTGSAATDTLTKGTKYSYKLNADTNGEWQSISIPLSEFKNNGNPVTIANIGCVTFQAGENGGLSNSASETKAMTAAELEAKARNGSIIFDNMELSNVGENVLNPNATPEPTEKPDNTTRTIDFDTYTLSHKQTWAGFNNNDKTYSDSIKSEITENGKEGCALELTYKAATWYAGEIFMSIPKEWAINKNSECLEFDAKGQGKIKISLETGEVVNGIRYGHTVTINTNDEWQKISVPLSEFVNNGNEVPLTDVVGMAFSAAESGNLDNNAEETKMMSADELEEKAVTGCVVIDNITLAEQDTTSPTAAPEATTQPTEISYVADFETADTKFASGKTWGGFKNKSNDYQDYIKAKWLQDGGVDGSTAFCVYYQSATYYAGEIFVPAPAVWTNNGAKGAEYLNFDYKGKGAVKISFSTGNTVDGTLTSGTRYTRRFELDSHGDWAKISVPLSEFVNGENIVNMTEIGTVTFQAAENANLDNNSDDTKAMSADELKEIARTGEIIFDNMTLSETEGKTTLFSSVKVTAEIDGKEITNLTNGDIKIKAIASDIEKDTNMVMIVAVYKENGVIDTVRMAGQKIIGDGELMLDLNVTDAEHQTMKVFIFDDFTNLHPIINVTNFL

SEQ ID NO: 25 - protein, 335 aa, unidentified
BMPTIRFVYTYSLLWWAERLCGKMYYIGIDLGGTNIAAGIVTEEGKIVVKDSVPTLSERPTDEIVTDMANLSKKLVQSIGIEMNEIKGIGIGCPGTIDFETGEIVYSNNIKINHYPLADKFKEHIPLPVKVDNDANCAALGEYKINRHCASVFALVTLGTGVGGGVIINGKVFRGFNGAAGELGHMTIVSGGKMCTCGKEGCLESYASATALISQTKDALETHKDTIMHGIVKKEGKISGRTAFEAAKQGDEVAKKVVSNYERYLADGIVSIENIFQPEIIAIGGGISKEGDYLIEPIREYVYNTGFNKHMTKTKIVAAQLFNDAGIIGAAMLAI

SEQ ID NO: 26 - protein, 588 aa, unidentified
MSEKFNNMSFRTKLLLSYIAVIILCIIIFGLTVFSSISRRFENEITDNNAQITGLAVNNMTNTMNNIEQILYSVQANSTIEKMLTASNPPSPYEEIAAIEQELSKIDPLKATVSRLSLYIENRTSYPSPFDSNVTASVYSKNEVWYKNTKELNGSTYWCVMDSSDANGLLCVARAFIDTRTHKILGIIRADVNLSQFTNDIAHISMNNTGKLFLVYENHIINTWNDSYINNFVNENEFFKAISADSDKPQLVQINKEKHIINHSRLKDSSLILVRASKLDDFNSDIHIIEKSMITTGIIALLVALIFIFIFTRWLTAPITKLIKHMERFENNYERIPIEITSHDEMGKLGESYNSMLNTIDSLITDVEDLYKKQKIFELKALQAQINPHFLYNTLDSIHWMARAHHAPDISKMVSALGTFFRHSLNKGNEYTTIENELNQISSYVSIQKIRFEDKFDVVYDIDENLLHCTIVKLTIQPLVENSIIHGFDEIEEGGMITIRIYPEDDYIFIDVIDNGSGADTNELNKAITHELDYNEPIEKYGLTNVNLRIQLYFDKTCGLSFKTNETGGVTATIKIKRKEPEYKTIDL

SEQ ID NO: 27 - protein, 337 aa, unidentified
MQCRGGNVMNFNIPDLGIIDGSSGFRNLPSTTDGRFTSGEDGVKHIVCTGDGKVEFVAFENQTLAYVNSALGYGAYYPLHPVNRNGKIKAVLMDLDGTSVRSEEFWIWIIEKTTASMLDDESFKLEESDIPFVSGHSVSEHLQYCIDKYCPGESLDKARNFYFDHVNREMKEIMEGRGRKNAFVPQEGLKEFLLALKAKGIKIGLVTSGLYEKAMPEILSAFRALDMGEPTDFYDAIISAGYPLRKGSVGTLGELSPKPHPWLYAETCAVGLGVGFDERGSVIAIEDSGAGVCSARIAGYTTIGLAGGNIKESGTMPMCSRYCNNLAEILDYIEEEA

SEQ ID NO: 28 - protein, 334 aa, unidentified
MFFSVLHMAIINIKGVKIVSELYPVRLIPVFKDYLWGGTKLKTVFNKKSELNILAESWELSANKDGQSIIANGKYQGYGLKEYIDIVGKEIVGTKGLALDDFPILIKFIDAKKNLSVQVHPDDEYATCHDGANAKTEMWYILDCNVGAYLYYGFKKDITKQEYQDAIRSNTITDVLNKVPVHKGDVFFIPAGTVHAIGAGILICEIQQNSNTTYRVYDYDRRDKDGNKRELHIREALESSNLKKSTYSNSVLDGDDIILTQCDYFTVRRLKVQNRVQLRIDKTSFHSLIITDGSGELYMGGEILKLNKGDSIFIPAQNNEYTVSGPCEIILSFL

SEQ ID NO: 29 - protein, 524 aa, unidentified
MNVKLLICDDEKIIREGLASLDWNTRGIEVVGTAKNGEVAFELFQKMLPDIVISDIKMPTKDGIWLSEQIHKISPNTKIIFLTGYNDFEYAQSAINNGVCQYLLKPIDEFELYEIVDKLTKEIHLEQQKAEKEIELRKTLRNSRYFLLNYLFNRAQYGILDFELFEISKKAAAMTTFVIRLDTDSTNYGMNFMIFEALIEHLPKTINFIPFFSNSDLVFICCFNEPEGESEQKLFSCCENLGDFIDTEFNVNYNIGIGIFTSEISELEASYTSALQALDYSDRLGQGNIIYINDIEPKSQLSAYQSKLIETYIKALKNNDEKQSKKSVKELFDVMERSDMNLYNQQRRCMSLILSISDALYDIDCDPTILFKNTDAWSLIRKTQSPAELKTFVENITDVVISYIESVQKQKAANIITQVKALVEKNYARDASLETVASQVFISPCYLSVIFKKETNITFKNYLIQTRIEKAKELLEKTDLKIYDIAEKVGYNNTRYFSELFQRICGKTPSQYRASHNPSMPQDI

SEQ ID NO: 30 - protein, 385 aa, unidentified
MSDKKPLYKQIMDKLKERIKSGDFEYDAPFVTEDRITKEYGVSRITAIRALEELEHDGLINRKRGSGSFVSKNAMSILGKDKEDNAAVTIHKKNRDISLVALVMPFDIKLGNMFKCFDGINSVLNKENCFVSIYNANRSVENEEKILRSLLEQGIDGVICYPVRGGRNFEVYNQFLVKKIPLVLIDNYIENMPMSYIVSDNSGGGKALCEYALEHGHKKIGFFCRGRVNETISIRDRYMGYAAALEEKGLGVNLDYVYANIDDKYEMLTEEERQQYGNVENYLKTIVNRMHEQGISCVLCQNDWVAIQVYNCCKALDISVPNEMCIMGFDNISELDEMDGGNKIITVEQNFFELGVKAGETVLREINGEMPGIKYIVPVKIAVRN

SEQ ID NO: 31 - protein, 268 aa, unidentified
MLVVLGASFTSESAISEFGFHAIPKEWSLDAYRYIITSKETILRAYGVTIFVTIVGTLMSTLVVALYAYPLSRKDFKYRKLFTFIAFFTMLFSGGTVAGYMVTTGILNLKNSIWVLIFPYVMNAWHVIVMRSFYSMSIPTAIIEAAKIDGANEYQIYFKIVLHISLPGLATIALFATLTYWNDWWLPLLYITEPQKYNLQYLLQSMISNIQNLTENSAQMGSANLLANVPKEGARMALCIIATLPILFVYPFFQKYFIQGLTVGSVKE

SEQ ID NO: 32 - protein, 294 aa, unidentified
MLSMCIPGLIFFILFNYLPMFGIIIAFKQYRYDLGIWASPWNGLKNFEFMFSSPDAWVITRNTIAYNLLFIFGGLVFNVAMAIGLSELRNKAVSKLCQTVVIMPHFLSYVIVSFLVLAFLHVENGLINRSLIPALGLEGVDWYSNPKYWPWILVIVNFWKTTGYGSVVYLAGIAGIDTSLYEAAKVDGASRWQQIRYITLPALVPLMVVLTILNVGKIFNSDFGLFYQVPLNTGALYPATNVISTYVYNMLMSAGTGSVGMASAAAFYQSIVGFILVMTTNFIVKKISPENALF

SEQ ID NO: 33 - protein, 515 aa, unidentified
MIMGKDETSEPLSKKKGDKIMRKKIAALLAMLMLGGVLTGCGGGNKVATGGEDPNVVPEDTYEINWYMQGMPQEDVASVEAAVNDYLKDKINATLKMHRLESNQYSKQLNTMIAAGEYFDIAWTTPGVLTYTANARNGAWLALDDYIDTYIPKTIEQLGEIADNARVDGKLYAIPTYKEMADSRGWTYRKDIAEKYNINMDNIKTFDELLPVLKMIKENEPNMQYPIDWGSDRTPEALMKYEEIAGTAVIFYDTDKYDGKVVNLVETPEYLEACKWDNKLYNEGLVKKDIMTATDFEQRLKDGKTFCYVDFLKPGKAKETSAKFDFELDQSTVSDIWQDNGAGTGSMLAVSRTSKNPERVLRFLELLNTDATLSNLINYGIEGKHYTKIDDNTITIPDDTSYTLQGYQWMQGNVFLNYLTEGESPDKVEALKAFNAEAKKPIDYGFKFDNTAVEAEIAACQTVKSEYRKQVIMGSMDPEPIMKEYAAKLKAAGIDKIIEEAQKQYDEFLANKNKQ

SEQ ID NO: 34 - protein, 385 aa, unidentified
MKKLLILFLLASVMLSMCSGCTVEKTVESAQAVTVLKVIKPNYISDFTQNIAEFNEANPDIQVKFIDAPTSTEKRHQLYVSALSGKDSSIDIYWINDEWTKEFVEQKYIKALDGEILLDNSRYIIDAQERFSVNDSFYAMPVGMDTDVIFYRSDKIHNVPETWDGIINLCRNSDFGLPIKLGLTTSDIQDMMYNIIEIKEAIGISYAETLNLYKEFIEEYKDIENYTDTIAAFKIGSAAMLMGNSSLWKKLNGDTSAVKGNIMVASLPNKNQFVRSYALAINSNSKNQEAAIRFLDFMNGKEQQRRLSRDTSLIPIIRELYDDEMILDANPHVKGIKQSVQNSSSFATVSINGENLKKLEEALIKFFNNEETSMNTGKIFEDLMQ

SEQ ID NO: 35 - protein, 1324 aa, Bacteroides intestinalis
MKQLITTLFIFIFLQPSWASLYRNYQVEDGLSHNSVWAVMQDKQGFLWFGTVDGLNRFDGNSFKIYKKLQGDSLSIGNNFIHCLKEDSHGHFLVGTKQGFYLFNRESETFSHVRLDNRSRGGDDTSINYIMEDPDGNIWLGCYGQGIYVLGPDLQVRKHYINKGNPGDIASNHIWCMVQDYNGVIWIGTDGGGLIRLDPKDERFTSIMHEKDLNLTDPTIYSLYCDMDNTIWVGTSISGLYRCNFRTGKVTNIVYPHRKILNIKAITAYSNNELVMGSDAGLIKVDCIQETISFINEGPAFDNITDKSIFSIAHDMEGGLWIGTYFGGVNYYSPYANKFAYYPGSSEEVSKSIISYFTEESSDKIWVGTKNEGLLLFNPAKISFETTHLQIDYHDIQALMMDNDKLWISVYGKGVSMVDVHSNTLLKRYSNDVGGPDLLTSNIVNVIFKSSKGQIFFGTPEGVDCLDAETKKINRLERTKGIPVKAIMEDYNGSIWFAAHMHGLLHLSADGTWESFTHMPEDSTSLMSNNVNCIHQDARYRIWVGSEGEGMGLFNPKTKKFEYILTENLGLPSNIIYAIQEDADGNIWVSTGGGLARIEPETRSICTFRYIEDLIKIRYNLNCALRGRDNHLYFGGTNGFIAFNPKDIQNNEYKPPICLTGFQISGNEVVPGIEGSPLKKSISMTQKIELESNQAAFSFDFVCLSYLSPAQNKYAYKLEGFDTDWHYVANGNNKAIYMNIPSGKYTFYVKGTNNDGVWCDTPIKVTVIVKRHFWLSNMMLLVYAILAISAFTLLIRRYNKRLDSINQDKMYKYKVEKEKEIYETKINFFTNMAHEIRTPLSLIVAPLENIISSGDGSQQTKSNLEIMKRNANRLLELVNQLLDFRKIEEDMFRLCFSKQNISEIVRNIHKRYVQYAKLKDIDIRLVEPEKDIACVVDKEAMEKVIGNLLSNAVKYANSLITINISTDNNLLTISVKDDGPGIKSEFIDKIFESFFQIENNAQRTGSGLGLALSKSLVTKHKGNIAASSDYGHGCTLTFTIPMDLPISISQLTEEYPEKEDISVQQTALSPVEGKLRIVLAEDNQELRSFLSNYLSDYLDVYEAQNGLEALQLVENENIDIIVSDILMPEMDGLELCKALKSNPAYSHLPFILLSARTDTATKIEGLNTGADVYMEKPFSSEQLRAQINSIINNRNSIRENFIKSPLDYYKQKSAEPNGNTEFIEKLNIIILDNLTNEKFSIDNLSEMFLMSRSNLHKKIKNIVGMTPNDYIKLIRLNQSAQLLATGKYKINEVCYLVGFNTPSYFSKCFYEHFGKLPKDFIVIE

SEQ ID NO: 36 - protein, 977 aa, synthetic construct
GSAEINYNRSVPLEVKGNKIVKQGTDEMVVLRGVNVPSMDWGMAENLYESMTMVYDCWGANLIRLPIHPKYWKDGSIWDGKNLTKEQYQKYIDDMVKAAQARGKYIILDCHRYVMPQQDDLDMLKELAVKYGNNSAVLFGLLNEPHDIKPTDIEKPTMEDQWEVWYNGGQIIVGGEEVTAIGHQQLLNEIRALGANNICIAGGLSWAFDISGLADGYNGRENGYRLIDTAEGHGVMYDSHAYPVKGTKSSWDTIIGPVRRVAPILIGEWGWDSSDNNISGGDCTSDIWMNQIMNWMDDTDNQYDGIPLNWTAWNLHMKSSPRMISSWDYKTTAYNGTYIKNRLQSYGNLPETQDGVYSTDFSTNDVFRGYKAPSGAASVSYSEANENIVVSHKPADWYATLNFPFDWDLNGIQTITMDISADTAETLNIGLYGSDMEEWTAPVKVDSTVKNITLSIDQLVRQGNQQTDGILNGAVSGIYIGSSTTETANNTKVVTMADEDNTAEYKINNYENGKVSVRKRTDAEDSASTVIVAFYDKNSVLTGISTANIRADEKGDEIIKAVNEPASYSCAEVFMWDSLNGMVPRCNPISNKVNITIDNIKIVKLAEPIYTATEYPHTDIGAESYIDVDNTDFASQSTTKGAASTSYFTCENAEVVGADGENTQAKYITYDRREGLYGGTVQFDLETVPSMDTKYFTISLKGSGTAQTINVNLGSEVSYNIALAEGDTDWHQYIFDISYGAQYPEDIAFVKLASNTKIESYFYADDFGFSKTKPERVIPNPEKTFIYDFATYNRNTAKYEAVISTMPGSNDDEIRAEKVDGGLDFETQALEITYSRNGNIPSKTMVVYSPSDFFKGNSNDDERTANRATLKADMEYMTDLVFYGKSTSDKNEKINVGVIDAANSMMTYTDTKEFTLTSQWQQFRVPFDEFKVLDGGSELDCSRVRGFVFSSAENSGEGSFMIDNITHTSVADIEWAE

SEQ ID NO: 37 - protein, 575 aa, Bacteroides salyersiae
MRHRVILFICVLQTLFAYAVGAETHFMLTLNEQWKFSTGDSSAWATTEFDDNQWGTISSRQYWEEQGYDGYDGYGWYRQHFMISEDWKPIVTNAGGLYIRYEFADDVDEVFVNGVSVGRMGEFPPEYKVIYGGMRKYKISPGLLRFGEENLIAIRVYDNGGAGGLKTENILLQSITPMDDLMLDIRCDDSDWVFENTETIDFRVRPKQPLAAGGEFNLVCSVTTDTYLPVDSFVYRVKGDFEQPVSFVPPAPGFYRITLYGEQQGVKSDFLKFNMGYCPEQIISPVDVEPDFDQFWETTLKELSEVVPDYRMTLLEEKSQGAKNIYRVEMYSLGNVRIEGYYAVPKQKGKFPSVISFLGYGSGGGFPRPDNLPGFCEFILSTRGQGIQLPVNTYGKWIVHGLEDKSQYYYRGAFMDLVRGIDFLCSRPEVDTEKIFAEGGSQGGAFTLAACALDRRICAAAPYIPFLSDFEDYFKIAPWPRSVFEEYLRSHEESSWDEIYRLLSYFDSKNLAPRITCPIIMGVGLQDNICPPHINFSGYNQVKSPKRYYIYYDKEHTVGKSWWTIRNNFFRSFCN

SEQ ID NO: 38 - protein, 737 aa, Bacteroides salyersiae
MKKLFKLFAFTCLAMSATAQNKTPIYLDETKPIEQRVEDALQRMTLEEKIKLCHAQSKFSSHGVPRLGIPELWMTDGPHGIREEVLWDEWKGAAWTSDSCIAFPALTCLAATWDLDMSALYGKSIGEEARFRGKDVLLGPGVNIYRTPLNGRNFEYMGEDPYLAAKMVVPYIKGVQQNGVAACVKHFALNNQEMYRGHINVEVSDRALHEIYLPAFKAAVLEGGTWSIMGAYNQYKGQHCCHNQYLLNDILKKDWNFDGTVISDWGGVHDTYQSAYYGLDLEMGTWTDGLSWGKTNAYNNYYMALPLLEKIKNGEIEENTVNDKVRRLLRMMFRTSMNTQKPWGSFGTEEHALAGRTIAENGIVLLKNENGLLPVDLSQIKKIAVIGENATKVMTLGGGSSSLKVKYEVSPLEGLKKRVGNAVELVYAPGYASPLTDKRDPRYIVLEGYRLPDAEKLTKEALEAAKNADIVLFFGGLNKNEHQDSEGTDRLNYHLPYGQDELIAQLSKVNKNIAVILISGNAVAMPWIKEVPSVLEAWFSGTESGNAIASVLVGDVNPSGKLPMTFAVRLEDYPAHTVGEYPGDSINVKYNEGIFVGYRWTDKHKIRSLFPFGHGLSYTTFQYGKALLSSSEMNEKEILTVTIPIKNTGKVKGKEIVQLYIGDEKSSLERPVKELKGFQKIELNPGEEKVVEFNITSNDLKFYDEAIQDWKAEQGKFNIFIGSSSTDIRAKTKFNLK

SEQ ID NO: 39 - protein, 756 aa, Bacteroides salyersiae
MGVSVFAADDGGALYLDAGRPVEQRVKDLMSRMTLEEKVGQMCQWVGLEHMRTASQDLTVDELSNNTARGFYPGITEEDVRQMTIDGKVGSFLHVLTVKEANQLQELAMKSRLKIPLIIGIDAIHGNAQVVGTTAYPTSIGQASMFDVGLVEEICRQTALEMRATGSQWTFNPNVEVARDPRWGRVGETFGEDPYLVSLLGVASVRGYQGDGFGKAENVLACAKHFIGGSQPINGTNGSPTDISERTLREVFLPPFKATVDAGVYSFMTAHNELNGIPCHANPWLMEDILRKEWGFDGFIVSDWMDIEHIHDLHRTAVDNKDAFYQSVDAGMDMHMHGPEFYEKVIELVKEGKLTEARIDESCRKILAAKFRLGLFEKSFTDEKAAKSVLFNEKHQATALEAARKSIVLLTNDGILPLDEAKYKNVFVTGMNADNQTILGDWALTQPDENVITVLEGLKLVSPDTKFSFVDLGWNIREMDKNKVEQAAKQAAKADLAIVAVGEYSLRTNWYDKTCGEDCDRSDINLAGLQQELVESILATGVPTVVVLVNGRQLGVEWIAGHANALVEAWEPGSLGGQAIAEILYGKVNPSGKLPVTVPRHVGQIQMIYNHKPSMYFHPYAIGESTPLFYFGYGLSYTEYAYSDLTVSSAQMSGDGSVEVSVKVTNTGTTDGEEIVQLYIRDLYSSATRPVKELKDFRRVPLRVGETKTVSFILPAGKLAFYDKKMDYTVEPGDYEIMVGASSRDEDLMKRIVNVK

SEQ ID NO: 40 - protein, 1045 aa, Bacteroides salyersiae
MEKKTKRIAFVLATMLCGWQMMLAQPVSPAPTPTRAANDVKAMFSDAYPEKFGKFQIDYDDWNSDKFLTTKTIVTPFGAADEVLKIEGLSTGSLQHNAQIALGTCNLSDMEYLHMDVYSPSENGIGEFSFYLVSGWSKTVSCNVWYNFDTKQEYDQWISIDIPMSTFKNGGLNLAEINVLRIARGKQGAPGTIVYVDNVYAYGKAVEPESDVKIVANGNANLTTDVPLISAPTPKVAAANVFNFFSDHYGDGKFDYAQSDYGDQKTVKSLITINDTEDQVFKIDNIVNGSKANVSIGSPNLSGVDMLHLDIFSPGNDQGIGEFDFALTDFGGNGNDAGIWLNITDKGWHGQWISIDIPLSKWTGAANMIRFRRGGKGSTGKLLYVDNVYAYKSESDDPKPVPDPTTVPVLTKDKSDVISIFCEQYEEPGYQDEFGIVSAGNWGQNAKQKDEFVEIVAGNQTLKLTSWDLFPFKVHKNSDVMDLSQMDYLHLSIYQNGALDENNKPVSVCIWINDKDNKVAQAPLLEVKQGEWTSVSFGMDYFKNKIDLSRVYVIRLKVGGYPTQDIYVDNIFGYKGDPIRPGQVTEPYVDECDQKIQDSTPGTLPPMEQAYLGVNLASASGGSNPGTFGHDYLYPKFEDLYYFKAKGIRLLRIPFRAPRLQHEVGGELDYDAGNTSDIKALAAVVKEAERLGMWVMLDMHDYCERNIDGVLYEYGVAGRKVWDSAKNTWGDWEAMDEVVLTKEHFADLWKKIATEFKDYTNIWGYDLMNEPKGININTLFDNYQAAIHAIREVDTKAQIVIEGKNYANAAGWEGSSDILKDLVDPVNKIVYQAHTYFDKNNTGTYKNSYDQEIGGNVEVYKQRIDPFIAWLEKNNKKGMLGEYGVPYNGHAQGDERYMDLIDDVFAYLKEKQLTSTYWCGGSMYDAYTLTVQPAKDYCTEKSTMKVMEKYIKDFDTSIPSSLVETNADGNAIVLYPNPVKDNLKITSESGIEQVIVFNMIGQKVSERNEKGTNIELNLEALGKGTYLVTVRLEDGNVVNRKIVKM

SEQ ID NO: 41 - protein, 413 aa, Bacteroides salyersiae
MSCVLVCAGVLLLLSGLRETDVVGTKKQLSYCDTQIKKTLDAIEGSGLMPRCIDTDATDWYKIDIYDWTSGFWPGILWYDYENTQNEEIRKAAIHYTESLVPLLDPEHPGDHDLGFQFYCSFGNAYRLTKDDKYKQVLLKGADKLAGFYDPRVGTILSWPGMVTEMNWPHNTIMDNMMNLELLFWAAKNGGNREYYGMAVSHAKVTKENQFRPDGSCYHVAVYDTIDGRFLKGVTNQGYSDSSLWARGQAWAIYGYTLVYRETGDKEYLRFAEKITDIYLKRLPEDYVPYWDFDDPAIPDAPRDASAAAIVASGLLELVQLEDNTEKAEEYRDAAVNMLLSLSSDAYQSGIKKPSFLLHCTGNLPGGYEIDASINYADYYYIEALTRYKKMQAGRDIVEKYPQATQKQVTIAM

SEQ ID NO: 42 - protein, 1029 aa, Bacteroides salyersiae
MKSHPLLILLIIIPTCLFAGNPDKVSLVDMFMGVKNSSNCVIGPQLPHGSVNPAPQTPNGGHNGYDENDVIRGFGQLHVSGIGWGRYGQVFISPQVGFKPGETEHDSPKSDEVATPYYYKVNLDRYKIKTEITPTHHSVYYRFTYPKSGNKNILLDMKHNIPQHIVPIVKGTFLGGNIEYDKASGLLTGWGEYAGGFGSAAPYKVFFAMRPDVKLKEVKVTDKGTKALYARLSLPEEAETVHLGIGVSLRSVENACKYLEQEIGARSFDEVKRVAKSAWEDVFATIDVKGGTQEEQRLFYTAMYHSFVMPRDRTGDNPRWTSGQPHLDDHFCVWDTWRTKYPLMMLVNESFVAKTVNSFIDRFAHDGECTPTFTSSLEWEMKQGGDDVDNIIADAFVKNLKGFDRQKAYELVKWNAFHARDSLYLKKGWIPETGARMSCSYTMEYAYNDDCGARIARIMKDDETADYLENRSQQWVNLFNPNLESHGFNGFVGPRKENGEWIGIDPALRYGPWVEYFYEGNSWVYTLFAPHQFSRLIRLCGGKEAMADRLTYGFEKELIELDNEPGFLSPFIFSHCDRPGQTAKYVDFIRKNHFSRATGYPENEDSGAMGAWYIFTSIGFFPNAGQDFYYLLPPAFSEVTLTMENGKKIDIKTVKSTPEVNYIESVSLNGKLLDRTWIRHAEIAEGATIVYHLTDKPGQWSISPFEASRREPQPFGVNLAGAEFFHKKMEGVGRFNKDYHYPTTDELDYWKSKGLTLIRLPFKWERIQRKLYGELNREEMDYIKFLLAEADKRDMQILIDMHNYGRRKDDGKDRIIGDSLSIDHFASAWGSISRELKDCKGLYGYGLINEPHDMLASTPWVGIAQAAIDSIRKNDAKNAIVVGGNHWSSAERWKLVSDDLKNLRDPSRNLIFEAHCYFDEDGSGIYRRSYEEEKAHPYIGVERMRPFVEWLKENDFRGLVGEYGVPADDERWLECLDNFLAYLSAEGVNGTYWAAGARWNRYILSVHPENDYRKDKPQMKVLMKYLRTQ

SEQ ID NO: 43 - protein, 1311 aa, Bacteroides salyersiae
MKHTILVLLGLALSFFPARAYHFRSYQVEDGLSHNSVWAVMQDSKGFMWFGTNDGLNRFDGKKIKVYRKIQGDSLSIGNNFIHCLKEDSRGRFLIGTKQGLYLFDDKLEKFRHIDLDKNIKDDVSINAIMEDPSGNIWLACHGYGLYVLTPELTTKKHYLSGSDPYSLPSNYIWSIVQDYYGNIWLGTVGKGLVHFDPKEEKFTQMTQAKELGIDDPVIYSLYCDIDNNIWIGTATSGLIRYTPRSQKATHYINHVFNIKSIIEYSDHELIMGSDKGLVKFDRTLESFDLINDDTSFDNMTDKSIFSIARDKEGSFWIGTYFGGVNYYSPAINRFQYCYNSPHNSSKKNIISGFAENENGDIWIGTHNDGLYLFNPKSLSFKKPYDIGYHDVQSILSDQDKLYASLYGKGIHILNIKNGQVSASANDIGINHTINSIAKTSKGQILFTSEGGVISMDASGTLKTLDYLTNTPVKDIAEDYDGSIWFATHSKGLIRLTSDNRWEVFVNNPDNPKSLPGNNVNCVFQDSKFHIWAGTEGEGLVRFNAKEQNFEPILNDQSGLPSNIIYSILDDSDGNLWVSTGGGLVKISSDLKNIKTFAYIGDIQRIQYNLNCALRASDNRLYFGGTNGFITFNPKEITDNPNKPVVMVTGFQIASKEITLSESSPLKETISATKEITLRHDQSTFSFDFVALSYLSPEQNRYAYILEGFDKEWHYTSDNKAMYMNIPPGTYVFRVKGTNNDGVWSDETADITVKIKPPFWLSNLMIGLYIVLAIGIILYFIRRYHRFIERKNQEKIFKYQTAKEKEMYESKINFFTNIAHEIRTPLSLIAAPLEKIILSGDGNEQTRNNLGMIERNANRLLELINQLLDFRKIEEDMFHFKFKRQNVVKIVEKVYKQYYQTAKFNKLEISLEAEKNDIECNVDSEAIYKIVSNLIANAIKYAKSQILITVKERSGNLEIKIKDDGTGIEKQYMEKIFEPFFQIQDKNNAVRTGSGLGLSLSQSLAMKHNGKISIESEYGKNCNFTLTIPIADGTEEEVQETEAAIPEKSEMPEQSVVEAGTRIIIVEDNTDMRTFLCESLNDNYTVFEAENGVQALEMVEKENIDIIISDIMMPEMDGLELCNRLKSDPAYSHLPLVLLSAKTDTSTKIEGLNQGADVYMEKPFSIEQLKAQISSIIENRNNLRKNFIKSPLQYFKQNTENNESADFVKKLNTIILENMSDEDFSIDSLSSQFAISRSNLHKKIKNITGMTPNDYIKLIRLNESARMLSTGKYKINEVCFLVGFNTPSYFSKCFFEQFKKLPKDFIQITNE

SEQ ID NO: 44 - protein, 723 aa, Bacteroides salyersiae
MKKQFSTLIALLIVGAAPLLGQETDPLNDPTNIDADLYLHAGFSQDSIRPDYSHTYYDNTNHKLVKGEDGIYSITVPLKKEQIVNKNMEVGIYTYAYSVIYGGKVNGSGNDAVKGSVGPVIADEPRLFELAEDRDVTFYAKKLNTGTADAPWYRTMFICDAQPLYLDGTELPLPGEDGVTRYVVDRGETSRRWEYKLSPIGRWSKTQDFMEDVIPAKWKSNEAYAFLPNGGWWLGGRFLLAYDYKKLSLEVGKLVDELQTPLFTVNGESIPENLGIVDELLLNGSVITFLKGYYANGGKDSYDPAFNTSIATVKLCWQIDELPAASFPLTNGEVVRDDNYNKTTEWTVSEADLFEGTTLPAGIHTLKVWYESEYLGDVLTSEVQSTSFEIEEIVVIPLENKGTAVDLILEGDWNPETFRTIIEEQAVRITTIDLTGVAGLTELPEMEGLNPNCLVYVNPDVVIAEGVDNVVVFDNEEGRAANILLTEGSDFNNVRLFTADRISYSHNFTADVWSTICLPFSADKGDVTVEEFTGADGEKVIFTGTSAIEANVPYLAKTSNSEVKTFTATDVQMSVTAEPAPVVPENGYAFHAGYRAVEGDAVVGLHLMNDVGTAFVKVADGNPEAAGVSAFHAYMQATVDELLTIVHGDDNPTGLGSTEDTGRLTIISHNGSVEIKTGKAQMIGLYALDGRLVKMVELSQGSNFVNGLDKGIYIMDCQKVVVK

SEQ ID NO: 45 - protein, 357 aa, Bacteroides salyersiae
MKKIITIAFLSFLYFVYGYASNHSMHPLKQIDYVLKQVKAQQEPYYSAYQQLIHDADSILKVSHHALVDFAVPGFYDKPEEHRANSLALQRDAYAAYCSALAYTLSGQQEYGEKACYFLNAWASTNEKYSEHDGVLVMTYSGSAFLMAAELMADDPLWSNKEKKDFRKWVKRIYQHAANTIRVHQNNWADWGRFGSLLAASFLNEKKEVAENVRLIKSDLFHKIATDGSMPEETRRGGNGIWYTYFSLAPMTGACWLVYNLTGENLFALEQDGTSIKKALDYMAYYNKHPKEWKWDKNPNTGKNEVWPENLLEAMANLYNDNSYVEYVKGKRPIIYRNHHFCWTFPTLMPTSFENYQ

SEQ ID NO: 46 - protein, 1025 aa, Bacteroides salyersiae
MISKDENIKRRIIGVLFFLCALSPALWAQSRIIKGEVLDPNGEPLIGVGVMIKNTTAGTITDVDGRYSIQVPDNNAVLSFSYVGYKRKEVKVGSQSVINISLEEESVLMDQVVIVGYGSQKKVNLTGAVAAISVDESLAGRSVANVSSALQGLMPGLSVSQSSGMAGNNSAKLLIRGLGTINSADPLIVVDDMPDADINRLNMNDIESITVLKDATASSVYGSRAANGVILVKTKSGKGLEKTQITFSGSYGWEKPTNTYDFISNYPRALTLQQISSSTNPGKNGENQNFKDGTIDQWLALGMIDDKRYPNTDWWDYIMRTGSIQNYNVSATGGSEKSNFYASVGYMKQEGLQINNDYDRYNARFNFDYKVMKNVNTGFRFDGNWSNFTYALDNGFTSDSNLDMQSAIAGIYPYDPVLDVYGGVMAYGEDPQAFNPLSFFTNQLKKKDRQELNASFYLDWEPVKGLVARVDYGLKYYNQFYKEADIPNRSYNFQTNSYGIREYVTENAGVTNQTSTGYKTLLNARLNYHTVFATHHDLNAMFVYSEEYWHDRYQMSYRQDRIHPSLSEIDAALSGTQSTSGNSSAEGLRSYIGRINYSAYGKYLLELNFRVDGSSKFQPGHQYGFFPSAALGWRFSEESFVKPYIGKWLASGKLRASYGKLGNNSGIGRYQQQEVLYQNNYMLDGSIAKGFVYSKMLNPDLTWESTGVFNLGLDLMFFDGKLAAEFDYYDRLTTGMLQKSQMSILLTGAYEAPMANLGTLRNRGFEANLTWRDRIADFTYSANFNISYNRTNLEKWGEFLDKGYVYIDMPYHFVYSQPDRGLAQTWTDSYNATPQGVAPGDVIRLDTNGDGRIDGNDKVAYTNFQRDMPTTNFALNLQMGWKGIDVSLLFQGSAGRKDFWNNKYTEINLPDKRYTSNWDQWNKPWSWENRGGEWPRLGGLVTNKTETDFWLQNMTYLRMKNLMIGYTFPKKWTRKCFIENLRIYGTAENLLTITGYKGLDPEKAANSQDLYPITKSYSIGVNLSF

SEQ ID NO: 47 - protein, 613 aa, Bacteroides salyersiae
MKRVYIKYIGLIAGMMMLFSSCADLLNQEPTVDLPATNYWKTESDAESALNGLVSDIRWLFNRDYYLDGMGEFVRVRGNSFLSDKGRDGRAYRGLWEINPVGYGGGWSEMYRYCYGGINRVNYVIDNVEKMIANASSEKTIKNLEGIIGECKLMRALVYFRLIMMWGDVPYIDWRVYDNSEVENLPRTPLAEVKDHILDDLLDAFKKLPEKATVEGRFSQPAALALRGKVLLYWASWNHYGWPELDTFTPSEEEARKAYKAAAEDFRTVIDDYGLTLFRNGEPGECDEPGKADKLPNYYDLFLPTANGDAEFVLAFNHGGTNTGQGDQLMRDLAGRSVENSQCWVSPRFEIADKYQSTITGDFCVPLVKLNPSSVPDARTRPNSAVNPESYKDRDYRMKASIMWDYEICQGLMSKKVTGWVPFIYKMWGSEVVINGETYMSYNTDGTNSGYVFRKFVRNYPGEERADGDFNWPVIRLADVFLMYAEADNAVNGPQPYAIELVNRVRHRGNLPVLASSKTSTPEAFFEAIKQERIVELLGEGQRAFDTRRWREIETVWCEPGGRGVKMYDTYGAQVAEFYVNQNNLAYERCYIFQIPESERNRNPNLTQNKPYR

(19) United States
(12) Patent Application Publication
(10) Pub. No.: US 2026/0002146 A1
(43) Pub. Date: Jan. 1, 2026

(54) ENZYMES AND MICROBES FOR XANTHAN GUM PROCESSING

(71) Applicant: THE REGENTS OF THE UNIVERSITY OF MICHIGAN, Ann Arbor, MI (US)

(72) Inventors: Matthew Ostrowski, Ann Arbor, MI (US); Eric Martens, Ann Arbor, MI (US)

(21) Appl. No.: 19/104,492

(22) PCT Filed: Aug. 23, 2023
(86) PCT No.: PCT/US2023/072742

§ 371 Date: Feb. 18, 2025

Related U.S. Application Data
(60) Provisional application No. 63/400,176, filed on Aug. 23, 2022.

(51) Int. Cl. C12N 9/88; A61K 38/00; C11D 3/386; C12N 9/18; C12N 9/24; C12N 15/74

(52) U.S. Cl. CPC C12N 9/88; C11D 3/38636; C12N 9/18; C12N 9/2402; C12N 15/74; C12Y 301/01006; C12Y 402/02012; A61K 38/00

(57) ABSTRACT

The present disclosure provides polypeptides having xanthan lyase activity, compositions, and uses thereof. The present disclosure also provides polynucleotides, expression vectors, host cells, and genetically modified organisms (e.g., bacteria) encoding xanthan lyases.

### CROSS-REFERENCE TO RELATED APPLICATIONS

This application claims the benefit of U.S. Provisional Application No. 63/400,176, filed Aug. 23, 2022, the content of which is herein incorporated by reference in its entirety.

### STATEMENT REGARDING FEDERALLY SPONSORED RESEARCH OR DEVELOPMENT

This invention was made with government support under DK118024 and DK125445 awarded by the National Institutes of Health. The government has certain rights in the invention.

### FIELD

The present disclosure provides xanthan lyase polypeptides, compositions, and uses thereof. The present disclosure also provides polynucleotides, expression vectors, host cells, and genetically modified organisms (e.g., bacteria) encoding xanthan lyases.

### SEQUENCE LISTING STATEMENT

The content of the electronic sequence listing titled UM-41182.601_SequenceListing.xml (Size: 74,122 bytes and Date of Creation: Aug. 22, 2023) is herein incorporated by reference in its entirety.

### BACKGROUND

Xanthan gum (XG) is an exopolysaccharide produced by Xanthamonas campestris that has been increasingly used as a food additive at concentrations of 0.05-0.5% (w/w) to increase stability, viscosity, and other properties of processed foods. Xanthan gum may also be included in foods as a replacement for gluten at up to gram quantities per serving. The polymer backbone is similar to cellulose, having β-1,4-linked glucose residues, however, xanthan gum contains trisaccharide branches on alternating glucose residues consisting of an α-1,3-mannose, β-1,2-glucuronic acid, and terminal β-1,4-mannose. Xanthan gum has also been used extensively in non-food industries. For example, the oil and gas industry uses xanthan gum in drilling fluid or mud for its rheological properties and in the secondary and tertiary recovery of petroleum.

### SUMMARY

Disclosed herein are polypeptides comprising a xanthan lyase. In some embodiments, the xanthan lyase comprises an amino acid sequence having at least 70% sequence identity to any of SEQ ID NOs: 1-4. In some embodiments, the xanthan lyase comprises an amino acid sequence selected from SEQ ID NOs: 1-4. In some embodiments, the xanthan lyase does not comprise SEQ ID NO: 5.

Also disclosed herein are polynucleotides comprising a nucleic acid sequence encoding a xanthan lyase polypeptide as disclosed herein, expression vectors comprising the polynucleotides operably linked with a promoter, and host cells comprising the polynucleotides or expression vectors.

Further disclosed herein are compositions comprising the xanthan lyase polypeptide disclosed herein. In some embodiments the compositions are cleaning compositions. In some embodiments the compositions are wellbore servicing compositions. The compositions may be liquids, gels, powders, granulates, pastes, sprays, bars, or unit doses. Also disclosed are methods comprising contacting an object or a surface with the polypeptide disclosed herein or a composition thereof.

Additionally, methods of degrading xanthan gum are disclosed. The methods comprise contacting xanthan gum or a composition comprising xanthan gum with the xanthan lyase disclosed herein or compositions thereof.

Further, genetically modified bacteria and compositions thereof are disclosed. In some embodiments, the genetically modified bacteria comprise at least one heterologous xanthan lyase. In some embodiments, the xanthan lyase comprises an amino acid sequence having at least 70% sequence identity to any of SEQ ID NOs: 1-5. In some embodiments, the xanthan lyase comprises an amino acid sequence selected from SEQ ID NOs: 1-5.

In some embodiments, the genetically modified bacteria further comprise one or more nucleic acids encoding one or more glycoside hydrolase, one or more carbohydrate uptake protein, one or more carbohydrate esterase, one or more polysaccharide lyase, or a combination thereof.

In some embodiments, the one or more glycoside hydrolase is a glycoside hydrolase family 5 enzyme, a glycoside hydrolase family 88 enzyme, a glycoside hydrolase family 94 enzyme, a glycoside hydrolase family 38 enzyme, a glycoside hydrolase family 92 enzyme, a glycoside hydrolase family 3 enzyme, or a combination thereof. In some embodiments, the glycoside hydrolase family 5 enzyme is derived from Ruminococcaceae UCG13. In some embodiments, the glycoside hydrolase family 5 enzyme independently comprises an amino acid sequence having at least 70% sequence identity to SEQ ID NO: 7, SEQ ID NO: 8, or SEQ ID NO: 36. In some embodiments, the glycoside hydrolase family 88 enzyme comprises an amino acid sequence having at least 70% identity to SEQ ID NO: 13, SEQ ID NO: 21, or SEQ ID NO: 41. In some embodiments, the glycoside hydrolase family 94 enzyme comprises an amino acid sequence having at least 70% identity to SEQ ID NO: 10. In some embodiments, the glycoside hydrolase family 38 enzyme independently comprises an amino acid sequence having at least 70% identity to SEQ ID NO: 11 or SEQ ID NO: 12. In some embodiments, the glycoside hydrolase family 92 enzyme independently comprises an amino acid sequence having at least 70% identity to SEQ ID NO: 22 or SEQ ID NO: 42. In some embodiments, the glycoside hydrolase family 3 enzyme independently comprises an amino acid sequence having at least 70% identity to SEQ ID NO: 18, SEQ ID NO: 38 or SEQ ID NO: 39.

In some embodiments, the one or more polysaccharide lyase family protein independently comprises an amino acid sequence having at least 70% identity to SEQ ID NO: 9, SEQ ID NO: 19, SEQ ID NO: 45.

In some embodiments, the one or more carbohydrate esterase independently comprises an amino acid sequence having at least 70% identity to SEQ ID NO: 14 or SEQ ID NO: 15.

In some embodiments, the one or more carbohydrate uptake protein independently comprises an amino acid sequence having at least 70% identity to SEQ ID NO: 16, SEQ ID NO: 17, SEQ ID NO: 46, or SEQ ID NO: 47.

In some embodiments, the genetic modified organism is a bacteria from the genus Bacteroides, Parabacteroides, Alistipes, Prevotella, Clostridium, Faecalibacterium, Eubacterium, Ruminococcus, Peptococcus, Peptostreptococcus, Bifidobacteria, Escherichia, or Lactobacillus.

In some embodiments, the bacterium is a gram-positive gut commensal bacteria. In some embodiments, the gram-positive gut commensal bacteria are selected from the genus Enterococcus, Staphylococcus, Lactobacillus, Clostridium, Peptostreptococcus, Peptococcus, Streptococcus, Bifidobacterium, or Faecalibacterium.

Also disclosed are methods for treating a disease or disorder in a subject. In some embodiments, the methods comprise administering to the subject a genetically modified bacterium, or composition thereof, to the subject. In some embodiments, the methods comprise administering to the subject a polypeptide comprising a xanthan lyase as described herein, or a composition thereof. In some embodiments, the disease or disorder comprises a gastrointestinal disease or disorder.

Other aspects and embodiments of the disclosure will be apparent in light of the following detailed description and accompanying figures.

### BRIEF DESCRIPTION OF THE DRAWINGS

FIGS. 1A and 1B are graphs of enzyme activity and kinetics from crude (FIG. 1A) and purified (FIG. 1B) samples, respectively, of exemplary xanthan lyases on xanthan gum oligosaccharides.

FIG. 2A is a schematic of the two PUL variants that were engineered into B. thetaiotaomicron and other Bacteroides to confer growth on the two different XGO substrates tested (XGO4 and XGO5). FIG. 2B, top panel is growth of native B. intestinalis possessing the Bi XGO-PUL and or engineered B. thetaiotaomicron strains on XGO4 and XGO5. Engineered B. thetaiotaomicron grows similarly to B. intestinalis on XGO4, indicating that the PUL is fully functional to promote growth on this tetrasaccharide substrate. However, only engineered B. thetaiotaomicron containing the additional PL8 gene added to the end of the major operon (susC-PL/CE-GH5-GH3 genes) is capable of growth on the more complicated XGO5, which is the substrate requiring PL8 activity. Positive and negative controls are shown at the bottom, consisting of either glucose (left) or no carbohydrate (right). The substrate key in the bottom right indicates the structure of XGO4 and XGO5 substrates and the site at which the key PL8 enzyme cleaves XGO5.

### DETAILED DESCRIPTION

The present disclosure provides a xanthan lyase which can degrade xanthan gum and xanthan gum oligosaccharides.

Section headings as used in this section and the entire disclosure herein are merely for organizational purposes and are not intended to be limiting.

### 1. DEFINITIONS

The terms “comprise(s),” “include(s),” “having,” “has,” “can,” “contain(s),” and variants thereof, as used herein, are intended to be open-ended transitional phrases, terms, or words that do not preclude the possibility of additional acts or structures. As used herein, comprising a certain sequence or a certain SEQ ID NO usually implies that at least one copy of said sequence is present in recited peptide or polynucleotide. However, two or more copies are also contemplated. The singular forms “a,” “and” and “the” include plural references unless the context clearly dictates otherwise. The present disclosure also contemplates other embodiments “comprising,” “consisting of” and “consisting essentially of,” the embodiments or elements presented herein, whether explicitly set forth or not.

For the recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the number 6.0, 6.1, 6.2, 6.3, 6.4, 6.5, 6.6, 6.7, 6.8, 6.9, and 7.0 are explicitly contemplated.

Unless otherwise defined herein, scientific, and technical terms used in connection with the present disclosure shall have the meanings that are commonly understood by those of ordinary skill in the art. For example, any nomenclatures used in connection with, and techniques of, cell and tissue culture, molecular biology, immunology, microbiology, genetics and protein and nucleic acid chemistry described herein are those that are well known and commonly used in the art. The meaning and scope of the terms should be clear; in the event, however of any latent ambiguity, definitions provided herein take precedent over any dictionary or extrinsic definition. Further, unless otherwise required by context, singular terms shall include pluralities and plural terms shall include the singular.

The term “bacterial artificial chromosome” or “BAC” as used herein refers to a bacterial DNA vector. BACs, such as those derived from E. coli, may be utilized for introducing, deleting, or replacing DNA sequences of non-human mammalian cells or animals via homologous recombination. E. coli can maintain complex genomic DNA as large as 500 kb or greater in the form of BACs (see Shizuya and Kouros-Mehr, Keio J Med. 2001, 50(1):26-30), with greater DNA stability than cosmids or yeast artificial chromosomes. In addition, BAC libraries of human DNA genomic DNA have more complete and accurate representation of the human genome than libraries in cosmids or yeast artificial chromosomes.

An “expression vector,” as used herein, refers to a linear or circular DNA molecule that comprises a polynucleotide encoding a polypeptide and is operably linked to control sequences that provide for its expression. The term “operably linked” means a configuration in which a control sequence (e.g., a promoter) is placed at an appropriate position relative to the coding sequence of a polynucleotide such that the control sequence directs expression of the coding sequence.

The term “host cell,” as used herein, refers to any cell type that is susceptible to transformation, transfection, transduction, or the like with a nucleic acid construct or expression vector comprising a polynucleotide of the present invention. The term “host cell” encompasses any progeny of a parent cell that is not identical to the parent cell due to mutations that occur during replication.

As used herein, “genetically modified” refers to an organism (e.g., a bacterium) which has a modification to introduce a nucleic acid that does not naturally occur in the organism or to introduce additional copies or modified forms of nucleic acids that naturally occur in the organism. The nucleic acid can be integrated in one or more copies into a genome or one or more copies of the nucleic acid can remain episomal, e.g., in a plasmid, phagemid or artificial chromosome.

A “peptide” or “polypeptide” is a linked sequence of two or more amino acids linked by peptide bonds. The polypeptide can be natural, synthetic, or a modification or combination of natural and synthetic. Peptides and polypeptides include proteins such as binding proteins, receptors, and antibodies. The proteins may be modified by the addition of sugars, lipids or other moieties not included in the amino acid chain. The terms “polypeptide” and “protein” are used interchangeably herein.

“Polynucleotide” or “oligonucleotide” or “nucleic acid,” as used herein, means at least two nucleotides covalently linked together. The polynucleotide may be DNA, both genomic and cDNA, RNA, or a hybrid, where the polynucleotide may contain combinations of deoxyribo- and ribo-nucleotides, and combinations of bases including uracil, adenine, thymine, cytosine, guanine, inosine, xanthine hypoxanthine, isocytosine and isoguanine. Nucleic acids may be obtained by chemical synthesis methods or by recombinant methods. Polynucleotides may be single- or double-stranded or may contain portions of both double stranded and single stranded sequence. The depiction of a single strand also defines the sequence of the complementary strand. Thus, a nucleic acid also encompasses the complementary strand of a depicted single strand. Many variants of a nucleic acid may be used for the same purpose as a given nucleic acid. Thus, a nucleic acid also encompasses substantially identical nucleic acids and complements thereof.

A “polysaccharide” or “oligosaccharide” is a linked sequence of two or more monomeric carbohydrates connected by glycosidic bonds. The polysaccharides can be natural, synthetic, or a modification or combination of natural and synthetic. polysaccharide may be modified by the addition of sugars, lipids or other moieties not included in the main chain of the polysaccharide.

As used herein, the terms “providing,” “administering,” and “introducing,” are used interchangeably herein and refer to the placement of the compositions of the disclosure into a subject by a method or route which results in at least partial localization of the composition to a desired site. The compositions can be administered by any appropriate route which results in delivery to a desired location in the subject.

The term “textile,” as used herein, refers to any textile material including yarns, yarn intermediates, fibers, non-woven materials, natural materials, synthetic materials, and any other textile material, fabrics made of these materials and products made from fabrics (e.g., garments and other articles). The textile or fabric may be in the form of knits, wovens, denims, non-wovens, felts, yarns, and towelling. The textile may be cellulose based such as natural cellulosics, including cotton, flax/linen, jute, ramie, sisal or coir, or manmade cellulosics (e.g., originating from wood pulp) including viscose/rayon, ramie, cellulose acetate fibers (tricell), lyocell or blends thereof. The textile or fabric may also be non-cellulose based such as natural polyamides including wool, camel, cashmere, mohair, rabbit and silk or synthetic polymer such as nylon, aramid, polyester, acrylic, polypropylene, and spandex/elastane, or blends thereof as well as blend of cellulose based and non-cellulose based fibers. Examples of blends are blends of cotton and/or rayon/viscose with one or more companion material such as wool, synthetic fibers (e.g., polyamide fibers, acrylic fibers, polyester fibers, polyvinyl alcohol fibers, polyvinyl chloride fibers, polyurethane fibers, polyurea fibers, aramid fibers), and cellulose-containing fibers (e.g., rayon/viscose, ramie, flax/linen, jute, cellulose acetate fibers, lyocell).

As used herein, “treat,” “treating,” and the like means a slowing, stopping, or reversing of progression of a disease or disorder when provided a composition described herein to an appropriate control subject. The term also means a reversing of the progression of such a disease or disorder to a point of eliminating or greatly reducing the cell proliferation. As such, “treating” means an application or administration of the compositions described herein to a subject, where the subject has a disease or a symptom of a disease, where the purpose is to cure, heal, alleviate, relieve, alter, remedy, ameliorate, improve, or affect the disease or symptoms of the disease.

A “subject” or “patient” may be human or non-human and may include, for example, animal strains or species used as “model systems” for research purposes, such a mouse model as described herein. Likewise, patient may include either adults or juveniles (e.g., children). Moreover, patient may mean any living organism, preferably a mammal (e.g., human or non-human) that may benefit from the administration of compositions contemplated herein. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. Examples of non-mammals include, but are not limited to, birds, fish, and the like. In one embodiment of the methods and compositions provided herein, the mammal is a human.

A “wellbore,” as used herein, refers to any hole drilled to aid in the exploration and/or recovery of natural resources, including oil, gas, or water. For example, a wellbore may be the hole that forms a well. A wellbore can be encased, for example by materials such as steel and cement, or it may be uncased.

Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting.

### 2. XANTHAN LYASE

The present disclosure provides a xanthan lyase and polypeptides comprising a xanthan lyase. The xanthan lyase has activity on xanthan gum and xanthan gum oligosaccharides previously treated with an enzyme having a glycoside hydrolase endoglucanase domain and activity. In some embodiments, the xanthan lyase comprises an amino acid sequence having at least 70% sequence identity to any of SEQ ID NOs: 1-4. In some embodiments, the xanthan lyase does not comprise SEQ ID NO: 5.

### a. Compositions

The present disclosure further provides compositions comprising xanthan lyases described herein. In some embodiments, the compositions are industrial compositions, agricultural compositions, personal care compositions, cleaning compositions, or compositions for use in manufacturing. In some embodiments, the compositions are pharmaceutical or therapeutic compositions.

The compositions may further comprise excipients. Excipients include any and all solvents, dispersion media, antibacterial and antifungal agents, isotonic and absorption delaying agents. Some examples of materials which can serve as excipients are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other compatible lubricants including, but not limited to, sodium lauryl sulfate and magnesium stearate, as well as coloring agents, releasing agents, preservatives, and antioxidants.

Techniques and formulations for pharmaceutical compositions may generally be found in “Remington's Pharmaceutical Sciences,” (Meade Publishing Co., Easton, Pa.). Therapeutic or pharmaceutical compositions must typically be sterile and stable under the conditions of manufacture and storage. The route or administration and the form of the composition usually dictates the type of carrier to be used.

The compositions may also comprise buffers (e.g., neutral buffered saline or phosphate buffered saline), carbohydrates (e.g., glucose, mannose, sucrose or dextrans), mannitol, antioxidants, bacteriostats, chelating agents such as EDTA or glutathione, solutes that render the formulation isotonic, hypotonic, or weakly hypertonic with the blood of a recipient, suspending agents, thickening agents and/or preservatives, commonly found in proteinaceous compositions.

The described xanthan lyases may be entrapped in microcapsules, in colloidal drug delivery systems (for example, liposome, albumin microspheres, microemulsions, nano-particles and nanocapsules), in macroemulsions, or in sustained-release preparation. The xanthan lyases, or nucleic acids thereof, may be in a liposome and combined with amphipathic agents such as lipids which exist in aggregated form as micelles, insoluble monolayers, liquid crystals, or lamellar layers which in aqueous solution. Suitable lipids for liposomal formulations include, without limitation, monoglycerides, diglycerides, sulfatides, lysolecithin, phospholipids, saponin, and bile acids. Preparation of such liposomal formulations is within the level of skill in the art.

### b. Nucleic Acids

The present disclosure also provides nucleic acids encoding the xanthan lyases and polypeptides described herein. In some embodiments, the nucleic acids encoding the xanthan lyases disclosed herein can be introduced into an expression vector, such that the expression vector comprises a promoter operably linked to the polynucleotides encoding the xanthan lyase described herein. The expression vector may allow expression of the xanthan lyases in a suitable expression system using techniques well known in the art, followed by isolation or purification of the expressed xanthan lyase. A variety of bacterial, yeast, plant, mammalian, and insect expression systems are available in the art and any such expression system can be used. Alternatively, a polynucleotide encoding a xanthan lyase can be translated in a cell-free translation system.

The selection of promoter will depend on the expression system being used. For example, suitable promoters for directing the transcription of the nucleic acid constructs of the present invention, especially in a bacterial host cell, are the promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene.

The expression vector may contain other control, selectable marker, or tag sequences. Control sequences include additional components necessary for the expression of a polynucleotide, including but not limited to, a leader, a polyadenylation sequence, a propeptide sequence, a signal peptide sequence, and a transcription or translation terminator. The control sequence(s) may be native or foreign to the nucleotide sequence encoding the polypeptide or native or foreign to each other. The control sequences may be provided with linkers for the purpose of introducing specific restriction sites facilitating ligation of the control sequences with the coding region of the nucleotide sequence encoding a polypeptide.

The selectable marker and any other parts of the expression construct may be chosen from those available in the art. In some embodiments, the selectable marker is a gene the product of which provides for biocide or viral resistance, resistance to heavy metals, prototrophy to auxotrophy, and the like and thereby permits easy selection of transformed, transfected, transduced, or the like cells. The selectable markers are primarily dictated by the host cell being used. For example, bacterial selectable markers commonly include markers that confer resistance to antibiotics, for example ampicillin, kanamycin, chloramphenicol, or tetracycline.

Various types of expression vectors are available in the art and include, but are not limited to, bacterial, viral, and yeast vectors. For example, the vector may include a plasmid, cosmid, bacteriophage, pl-derived artificial chromosome (PAC), bacterial artificial chromosome (BAC), yeast artificial chromosome (YAC), or mammalian artificial chromosome (MAC). The various vectors may be selected based on the size of polynucleotide inserted in the construct.

### c. Host Cells

Also provided is a host cell comprising the xanthan lyase or the expression vectors described herein. The host cell may be any cell useful in the recombinant production of a xanthan lyase of the present invention, e.g., a prokaryote or a eukaryote. The prokaryotic host cell may be any Gram-positive or Gram-negative bacterium. Gram-positive bacteria include, but are not limited to, Bacillus, Clostridium, Enterococcus, Geobacillus, Lactobacillus, Lactococcus, Oceanobacillus, Staphylococcus, Streptococcus, and Streptomyces. Gram-negative bacteria include, but are not limited to, Campylobacter, E. coli, Flavobacterium, Fusobacterium, Helicobacter, Ilyobacter, Neisseria, Pseudomonas, Salmonella, and Ureaplasma. The host cell may also be a eukaryote, such as a mammalian, insect, plant, or fungal cell.

In some embodiments, the host cell is a gastrointestinal microbiota (gut flora) microorganism that is modified to express and/or secrete the xanthan lyase described herein. Such host cells find use in populating gastrointestinal systems of host organisms (e.g., people, livestock, etc.) to regulate (e.g., increase) that ability of the host organism to digest or otherwise process xanthan gum. These host cells find particular use in subject that have a high dietary intake of xanthan gum (e.g., human subject on a low gluten or gluten-free diet). Such host cells may be introduced into a subject by any suitable methodology including, but not limited to, administration of probiotics containing the host cells and fecal microbiota transplantation.

Host cells that find use in such application include, for example, bacteria belonging to the genera Bacteroides, Clostridium, Faecalibacterium, Eubacterium, Ruminococcus, Peptococcus, Peptostreptococcus, and/or Bifidobacterium.

In some embodiments, the host cell is in the genera Bacteroides, including but not limited to, B. acidifaciens, B. amylophilus, B. asaccharolyticus, B. barnesiae, B. bivius, B. buccae, B. buccalis, B. caccae, B. capillosus, B. capillus, B. cellulosilyticus, B. chinchilla, B. clarus, B. coagulans, B. coprocola, B. coprophilus, B. coprosuis, B. corporis, B. denticola, B. disiens, B. distasonis, B. dorei, B. eggerthii, B. endodontalis, B. faecichinchillae, B. faecis, B. finegoldii, B. fluxus, B. forsythus, B. fragilis, B. furcosus, B. galacturonicus, B. gallinarum, B. gingivalis, B. goldsteinii, B. gracilis, B. graminisolvens, B. helcogenes, B. heparinolyticus, B. hypermegas, B. intermedius, B. intestinalis, B. johnsonii, B. levvi, B. loescheii, B. macacae, B. massiliensis, B. melaninogenicus, B. merdae, B. microfusus, B. multiacidus, B. nodosus, B. nordii, B. ochraceus, B. oleiciplenus, B. oralis, B. oris, B. oulorum, B. ovatus, B. paurosaccharolyticus, B. pectinophilus, B. pentosaceus, B. plebeius, B. pneumosintes, B. polypragmatus, B. praeacutus, B. propionicifaciens, B. putredinis, B. pyogenes, B. reticulotermitis, B. rodentium, B. ruminicola, B. salanitronis, B. salivosus, B. salyersiae, B. sartorii, B. splanchnicus, B. stercorirosoris, B. stercoris, B. succinogenes, B. suis, B. tectus, B. termitidis, B. thetaiotaomicron, B. uniformis, B. ureolyticus, B. veroralis, B. vulgatus, B. xylanisolvens, B. xylanolyticus, B. zoogleoformans, and any combination thereof.

### 3. GENETICALLY MODIFIED BACTERIA

The present disclosure provides genetically modified bacteria encoding at least one heterologous xanthan lyase. Xanthan lyases include any enzyme capable of cleaving the β-D-mannosyl-β-D-1,4-glucuronosyl bond of xanthan gum or xanthan gum oligosaccharides. In some embodiments, the xanthan lyase is an enzyme having EC number: EC 4.2.2.12.

Xanthan lyases are known in the art, for example, two xanthan lyases isolated from Paenibacillus alginolyticus XL-1 (Ruijssenaars et al., (1999) Appl. Environ. Microbiol. 65(6): 2446-2452 and Ruijssenaars et al., (2000) Appl. Environ. Microbiol. 66(9): 3945-3950, both incorporated by reference herein in their entirety), a xanthan lyase from Paenibacillus nanensis and variants thereof (Jensen et al., (2019) Cell Chem. Biol. 26, 191-202.e6, incorporated by reference herein in its entirety), a Bacillus sp. GL1 xanthan lyase (Hashimoto et al., (2003) J. Biol. Chem. 278, 7663-7673, incorporated by reference herein in its entirety), a xanthan lyase from Microbacterium sp. strain XT11 (Yang, et al., (2014) The Scientific World Journal 2014, incorporated by reference herein in its entirety), and from various other bacterial species (Sutherland, I. W., (1987) Microbiology 133, no. 11, 3129-3134, incorporated by reference herein in its entirety).

In some embodiments, the xanthan lyase comprises an amino acid sequence having at least 70% sequence identity to any of SEQ ID NOs: 1-5. In some embodiments, the xanthan lyase comprises an amino acid sequence of selected from SEQ ID NOs: 1-5.

The genetically modified bacterium may encode additional genes used in xanthan processing and degradation. In some embodiments, the genetically modified bacterium further comprises one or more nucleic acids encoding one or more glycoside hydrolase, one or more carbohydrate uptake protein, one or more carbohydrate esterase, one or more polysaccharide lyase, or a combination thereof.

Glycoside hydrolases are enzymes that catalyze the hydrolysis of the glycosidic linkage of glycosides, often leading to formation of sugar hemiacetal or hemiketal products. Glycoside hydrolases are also referred to as glycosidases, and sometimes also as glycosyl hydrolases. The glycoside hydrolases have been classified into more than 100 families available through the Carbohydrate Active enZyme database. Each family contains proteins that are related by sequence, and by extension, tertiary structure. In some embodiments, the one or more glycoside hydrolase is, or is derived from, a glycoside hydrolase family 5 enzyme, a glycoside hydrolase family 88 enzyme, a glycoside hydrolase family 94 enzyme, a glycoside hydrolase family 38 enzyme, a glycoside hydrolase family 92 enzyme, a glycoside hydrolase family 3 enzyme, or a combination thereof.

In some embodiments, the one or more glycoside hydrolase is, or is derived from, a glycoside hydrolase family 5 enzyme. In some embodiments, glycoside hydrolase family 5 enzyme comprises a glycosyl hydrolase 5 endoglucanase domain having an amino acid sequence having at least 70% (e.g., 70%, 75%, 80%, 85%, 90%, 95%, or 95%) sequence identity to SEQ ID NO: 6. In some embodiments, the xanthan-utilization gene or gene locus comprises a gene encoding a glycoside hydrolase family 5 enzyme derived from Ruminococcaceae UCG13. In some embodiments, the glycoside hydrolase family 5 enzyme may comprise an amino acid sequence having at least 70% sequence identity to SEQ ID NO: 7, 8, or 36.

In some embodiments, the glycoside hydrolase is from the glycoside hydrolase family 88 (GH88). In some embodiments, the glycoside hydrolase family 88 protein comprises an amino acid sequence having at least 70% identity to SEQ ID NO: 13, 21, or 41.

In some embodiments, the glycoside hydrolase is from the glycoside hydrolase family 94 (GH94). In some embodiments, the glycoside hydrolase family 94 protein comprises an amino acid sequence having at least 70% identity to SEQ ID NO: 10.

In some embodiments, the glycoside hydrolase is from the glycoside hydrolase family 38 (GH38). In some embodiments, the glycoside hydrolase family 38 protein comprises an amino acid sequence having at least 70% identity to SEQ ID NO: 11 or 12.

In some embodiments, the glycoside hydrolase is from the glycoside hydrolase family 92 (GH92). In some embodiments, the glycoside hydrolase family 92 protein comprises an amino acid sequence having at least 70% identity to SEQ ID NO: 22 or 42.

In some embodiments, the glycoside hydrolase is from the glycoside hydrolase family 3 (GH3). In some embodiments, the glycoside hydrolase family 3 protein comprises an amino acid sequence having at least 70% identity to SEQ ID NO: 18, 38, or 39.

Carbohydrate uptake proteins include any proteins or enzymes necessary for the import of carbohydrates, including xanthan oligosaccharides, into the bacterial cell. Carbohydrate uptake proteins may include, but are not limited to, carbohydrate binding proteins and carbohydrate transporters. The carbohydrate uptake proteins may include members of the starch utilization system (Sus) of Bacteroides. The Sus includes the requisite proteins for binding and processing carbohydrates at the surface of the cell and, the subsequent oligosaccharide transport across the membrane for further degradation. All mammalian gut Bacteroidetes possess analogous Sus-like systems that target numerous diverse glycans. The carbohydrate uptake protein may include SusC or SusD or homologs or variants thereof from Bacteroides known in the art (See, for example, Xu, et al., PLoS Biol. 2007 July; 5(7): e156 and Foley, et al., Cell Mol Life Sci. 2016 July; 73(14): 2603-2617, both incorporated by reference herein in their entirety. In some embodiments, the one or more carbohydrate uptake proteins independently comprise an amino acid sequence having at least 70% identity to SEQ ID NOs: 16, 17, 46, or 47. In some embodiments, the bacterium comprises two or more carbohydrate uptake proteins, for example, of SEQ ID NOs: 16 and 17 or SEQ ID NOs: 46 and 47.

Polysaccharide lyases (or eliminases) are a class of enzymes that act to cleave certain activated glycosidic linkages present in polysaccharides. These enzymes act through an eliminase mechanism, rather than through hydrolysis, resulting in unsaturated oligosaccharide products. Polysaccharide lyases are endogenous to various microorganisms, bacteriophages, and some eukaryotes. The polysaccharide lyases have been classified into approximately 40 families available through the Carbohydrate Active enZyme (CAZy) database. In some embodiments, the polysaccharide lyase family protein comprises a polysaccharide lyase family 8 protein. In some embodiments, the polysaccharide lyase family protein comprises a polysaccharide lyase family 2 protein. In some embodiments, the bacterium comprises a polysaccharide lyase family 8 protein and a polysaccharide lyase family 2 protein. In some embodiments, the polysaccharide lyase family protein comprises an amino acid sequence having at least 70% identity to SEQ ID NO: 9, 19, or 45.

Carbohydrate esterases are a group of enzymes which release acyl or alkyl groups attached by ester linkage to carbohydrates. The carbohydrate esterases catalyze deacetylation of both O-linked and N-linked acetylated saccharide residues (esters or amides). The carbohydrate active enzyme database has 16 classified families of carbohydrate esterases. In some embodiments, the carbohydrate esterase used herein is capable of deacetylating xanthan oligosaccharides. The heterologous xanthan-utilization gene or gene locus may include one or more carbohydrate esterases. In some embodiments, the one or more carbohydrate esterases comprise an amino acid sequence having at least 70% identity to SEQ ID NOs: 14 or 15. In some embodiments, the heterologous xanthan-utilization gene or gene locus includes two carbohydrate esterases, for example, one having SEQ ID NO: 14 and one having SEQ ID NO: 15.

The genetically modified bacterium may further comprise additional genes encoding proteins and enzymes used in xanthan processing and degradation including, but not limited to, glucokinases, mannose-6-phophate isomerases, phosphoglucomutases, environmental sensors, and signaling proteins (e.g., response regulators). For example the gene locus may further comprise a glucokinase protein having an amino acid sequence having at least 70% identity to SEQ ID NO: 23 or 25, a transporter protein having an amino acid sequence having at least 70% identity to SEQ ID NO: 31-34, a transcriptional regulator having an amino acid sequence having at least 70% identity to SEQ ID NO: 30, a response regulator having an amino acid sequence having at least 70% identity to SEQ ID NO: 29, an isomerase having an amino acid sequence having at least 70% identity to SEQ ID NOs: 27 or 28, a kinase having an amino acid sequence having at least 70% identity to SEQ ID NO: 26, a carbohydrate-binding module protein (e.g. Carbohydrate-binding module family 11 protein) having an amino acid sequence having at least 70% identity to SEQ ID NO: 24, and/or an environmental sensor (e.g. hybrid two-component system (HTCS) protein) having an amino acid sequence having at least 70% identity to SEQ ID NO: 35 or 43.

In some embodiments, the genetically modified bacterium is from the genera Bacteroides, Clostridium, Faecalibacterium, Eubacterium, Ruminococcus, Peptococcus, Peptostreptococcus, Bifidobacteria, Escherichia, and/or Lactobacillus.

In some embodiments, the genetically modified bacterium is a gram-positive gut commensal bacteria. The gram-positive gut commensal bacteria may be from the genera Enterococcus, Staphylococcus, Lactobacillus, Clostridium, Peptostreptococcus, Peptococcus, Streptococcus, Bifidobacterium, and/or Faecalibacterium. In some embodiments, the gram-positive gut commensal bacteria may be Lactobacillus reuteri or Clostridium scindens.

In some embodiments, the polynucleotide encoding at least one heterologous xanthan lyase is on a plasmid, a bacterial artificial chromosome, or in the genetically modified bacterium's genome. In some embodiments, the one or more nucleic acids (e.g., the one or more nucleic acids encoding additional genes used in xanthan processing and degradation) are on a plasmid, a bacterial artificial chromosome, or in the genetically modified bacterium's genome. In some embodiments, at least one or all of the one or more nucleic acids (e.g., the one or more nucleic acids encoding additional genes used in xanthan processing and degradation) are on the same plasmid or bacterial artificial chromosome as the polynucleotide encoding at least one heterologous xanthan lyase.

Also provided are compositions comprising the genetically modified bacteria described herein. In some embodiments, the composition is a pharmaceutical composition (e.g., probiotic composition) further comprising excipients and/or pharmaceutically acceptable carriers. The excipients and/or pharmaceutically acceptable carriers may facilitate delivery of the genetically modified bacteria to a subject, for example a subject's gastro-intestinal tract, in a viable and metabolically-active condition, for example in a condition capable of colonizing and/or metabolizing and/or proliferating in the gastrointestinal tract.

The choice of excipients or pharmaceutically acceptable carriers will depend on factors including, but not limited to, the particular mode of administration, the effect of the excipient on solubility and stability, and the nature of the dosage form.

Excipients and carriers may include any and all solvents, dispersion media, coatings, and isotonic and absorption delaying agents. Some examples of materials which can serve as excipients and/or carriers are sugars including, but not limited to, lactose, glucose and sucrose; starches including, but not limited to, corn starch and potato starch; cellulose and its derivatives including, but not limited to, sodium carboxymethyl cellulose, ethyl cellulose and cellulose acetate; powdered tragacanth; malt; gelatin; talc; excipients including, but not limited to, cocoa butter and suppository waxes; oils including, but not limited to, peanut oil, cottonseed oil, safflower oil, sesame oil, olive oil, corn oil and soybean oil; glycols; including propylene glycol; esters including, but not limited to, ethyl oleate and ethyl laurate; agar; buffering agents including, but not limited to, magnesium hydroxide and aluminum hydroxide; alginic acid; pyrogen-free water; isotonic saline; Ringer's solution; ethyl alcohol, and phosphate buffer solutions, as well as other non-toxic compatible lubricants including, but not limited to, sodium lauryl sulfate and magnesium stearate, as well as coloring agents, releasing agents, coating agents, sweetening, flavoring and perfuming agents, preservatives and antioxidants. The compositions of the present invention and methods for their preparation will be readily apparent to those skilled in the art. Techniques and formulations may be found, for example, in Remington's Pharmaceutical Sciences, 19th Edition (Mack Publishing Company, 1995). The composition can comprise additional components, such as vitamins, minerals, carbohydrates, and a mixture thereof.

The composition may take on many forms. In some embodiments, the composition comprises encapsulating (e.g., in tablets, caplets, microcapsules) the genetically modified bacteria for enhanced delivery and survival in the gastric and/or gastrointestinal tract of a subject. In some embodiments, the composition is a foodstuff including liquids (e.g., drinks), semi-solids (e.g., jellies, yogurts, puddings, smoothies, and the like) and solids.

### 4. METHODS

The present disclosure provides methods of degrading xanthan gum. In some embodiments, the methods comprise contacting the xanthan gum or xanthan gum oligosaccharides with a xanthan lyase as disclosed herein, or a composition comprising thereof.

In some embodiments, disclosed methods comprise administering a xanthan lyase as disclosed herein, to a subject. In some embodiments, the administration is oral such that the xanthan lyase is made available in the digestive tract (e.g., mouth, stomach, small intestine, large intestine, etc.) at a concentration sufficient to digest xanthan gum present in the subject. In some such embodiments, the xanthan lyase is provided in a capsule or other carrier that releases the peptides at a desired location in the digestive tract.

In some embodiments, the xanthan lyase is expressed in a host cell which is provided to the subject. In some embodiments, the host cell is a gastrointestinal microbiota microorganism. The polypeptide may be transiently or stably expressed in the microorganism. A nucleic sequence encoding the xanthan lyase may be under the control of a promoter that provides optimized expression (e.g., overexpression) of the xanthan lyase. In some embodiments, the promoter is an inducible promoter that permits control over the timing and/or level of expression. In some embodiments, the xanthan lyase is encoded by a nucleic acid sequence that further encodes a signal sequence such that the translated xanthan lyase contains the signal sequence. Signal sequences find use, for example to increase extracellular secretion of the xanthan lyase.

One of skill in the art can monitor the reaction and the products produced by using any carbohydrate analysis method known in the art, including but not limited to, liquid chromatography-mass spectrometry (LC-MS), thin layer chromatography (TLC), gas chromatography (GC), high performance liquid chromatography (HPLC), and quantitative size exclusion or molecular sieve chromatography.

The xanthan lyases described herein, and compositions thereof, may be used in any application which requires or is beneficial to degrade or remove xanthan gum, including for example, cleaning compositions, well treatment compositions, and wellbore servicing compositions. The present disclosure further provides compositions comprising the xanthan lyases described herein and methods of use thereof. The composition may take on any desired form (e.g., liquid, gel, powder, granulate, paste, spray, bar, unit dose, microcapsule, and the like

Accordingly, the present disclosure provides methods of cleaning utilizing a xanthan lyase, as described herein, or compositions thereof. The methods comprise contacting an object or a surface with the polypeptides or compositions thereof. In some embodiments, the methods further comprise at least one or both of rinsing the object or surface and drying the object or surface. In some embodiments, the object or surface comprises a textile, a plate, tile, dishware, silverware, glass, a wellbore, or wellbore filter cake.

The process of contacting can be done in a variety of different ways, depending on the composition and the object being cleaned. For example, the composition can be diluted into water to for a cleaning solution which is then contacting the surface or object as commonly done in dishwashing, laundry, and floor cleaning applications. The composition may be directly applied to the surface or object as a spray, liquid, foam, or solid, as is common for fabric spot treatments and hard surface cleansers. The contacting may be carried out for any period of time and may include a soaking period in which the object or surface remains in contact with the composition for a period of time, for example, for at least about 1 hour, at least about 4 hours, at least about 8 hours, at least about 16 hours, or at least about 24 hours.

Thus, in some embodiments, the composition is a cleaning composition. Cleaning compositions include, but are not limited to, detergent compositions (e.g., liquid and/or solid laundry detergents and dish washing detergents); hard surface cleaning formulations, such as for glass, wood, ceramic and metal counter tops, floors, tables, walls, and windows; carpet cleaners; oven cleaners; fabric fresheners; fabric softeners; and textile and laundry pre-treaters.

The cleaning compositions may comprise one or more additional enzymes, such as proteases, amylases, lipases, cellulases, endoglucanases, xyloglucanases, pectinases, pectin lyases, peroxidaes, catalases, mannanases, redox enzymes, or any mixture thereof. The cleaning compositions may also comprise one or more components selected from surfactants, builders, chelating agents, bleaching components (e.g., precursors, activators, catalysts), antibacterial agents, antifungal agents, polymers, degreasers, corrosion inhibitors, stabilizers, antioxidants, colorants, fragrances, foaming agents, emulsifiers, moisturizers, abrasives, binders, viscosity controlling agents, and pH controlling agents. One of skill in the art is capable of selecting the additional components based on the desired functionality of the composition.

Thus, in some embodiments, the composition is a well treatment composition or a wellbore servicing composition. Xanthan gum is commonly used for increasing the viscosity of drilling fluids (e.g., drilling mud, drill-in fluids, or completion fluids). Compositions comprising a xanthan lyase, such as those disclosed herein, may be used to decrease viscosity of the fluids and/or clean well bores and wellbore filter cakes. Filter cakes are coatings on the walls of the wellbore that limit drilling fluid losses, preserve the integrity of the drilling fluid, prevent formation damage, and provide a balanced density. To form a filter cake, the drilling fluid is often intentionally modified with a weighting material including barite, iron oxide, or calcium carbonate and some particles of a size slightly smaller than the pore openings of the formation. It is these particles which may contain xanthan gum and improve viscosity and emulsification properties of the drilling fluid.

The well treatment composition or wellbore servicing composition may also comprise one or more additional components selected from chelating agents; converting agents (carbonate, nitrate, chloride, formate, or hydroxide salts); other polymer removal agents (persulfate salt, a perborate salt, a peroxide salt, and other enzymes, for example, amylases, glucanases, mannanases, cellulases, oxidoreductases, hydrolases, lyases); organic solvents; surfactants; binders; an aqueous liquid, which may be water, brine, seawater, or freshwater; fragrances; colorants; dispersants; pH control agents or acidifying agents; water softeners or scale inhibitors; bleaching agents; crosslinking agents; antifouling agents; antifoaming agents; anti-sludge agents; corrosion inhibitors; viscosity modifying agents; friction reducers; freeze point depressants, iron-reducing agents; and biocides. One of skill in the art is capable of selecting the additional components based on the desired functionality of the components. Exemplary compositions and methods of using well treatment or wellbore servicing compositions can be found in U.S. Pat. Nos. 5,881,813, 6,110,875, and 9,890,321 and U.S. Patent Publications 2020/0131432 and 2020/0115609; each incorporated herein by reference in its entirety.

The disclosure also provides, a method of treating a disease or disorder comprising administering a therapeutically or prophylactically effective dose of the genetically modified bacteria or compositions thereof, as described herein, to a subject in need thereof. The specific dose level may depend upon a variety of factors including the age, body weight, and general health of the subject, time of administration, and route of administration. An “effective amount” is an amount that is delivered to a subject, either in a single dose or as part of a series, which achieves a medically desirable effect. For therapeutic purposes, and effect amount is the quantity which, when administered to a subject in need of treatment, improves the prognosis and/or state of the subject and/or that reduces or inhibits one or more symptoms to a level that is below that observed and accepted as clinically diagnostic or clinically characteristic of the disease or disorder. For prophylaxis purposes, an effective amount is that amount which induces a protective result without significant adverse side effects.

The frequency of dosing the effective amount can vary, but typically the effective amount is delivered daily, either as a single dose, multiple doses throughout the day, or depending on the dosage form, dosed continuously for part or all of the treatment period.

The genetically modified bacteria may be administered at about 10⁴ to about 10¹⁰ cfu per dose, about 10⁵ to about 10⁹ cfu per dose, about 10⁵ to about 10⁷ cfu per dose, or about 10⁹ cfu per dose.

The disease or disorder may comprise a gastrointestinal disease or disorder including diseases and disorders that cause inflammation in the gastrointestinal system including, but not limited to, Irritable Bowel Syndrome, diarrhea, Crohn's disease, ulcerative colitis, and gluten intolerance or Celiac's disease. The treatment may be combined with gluten-free or low carbohydrate diets that are high in xanthan gum.

In some embodiments, the administration is oral. The genetically modified bacteria may be administered with food (e.g., concomitantly with food, within an hour of before or after consuming food).

### 5. EXAMPLES

### Example 1

### Expression and Purification of Xanthan Lyase

Xanthan gum oligosaccharide preparation: 1500 mL of 25 mg/mL xanthan gum was prepared by dissolving in water and autoclaving for 40 minutes. Phosphate buffer saline (300 mL) and ˜50 mg of a glucoside hydrolase XGD26-15 (SEQ ID NO: 7) were added to the sterilized xanthan gum and incubated at 37° C. for 1 week. An additional ˜25 mg of XGD26-15 was added to insure full digestion of xanthan gum to xanthan gum oligosaccharides. Crude digested xanthan gum oligosaccharides were centrifuged at 4° C. and >10,000 g to pellet precipitated protein and other insoluble material. Supernatants were filter sterilized using a 0.2 micron vacuum filter.

Plasmid constructs comprising modified xanthan lyases XGD153-I, XGD153-J, XGD153-K, and XGD153-L (SEQ ID NOs: 1-4, respectively) were transformed into HI-Control BL21 (DE3) cells and single colonies were inoculated in 5 ml overnight Luria-Bertani broth cultures at 37° C. Cultures (5 ml) were used to inoculate 1 1 of terrific broth (TB) with selective antibiotic (kanamycin), grown to OD600 ˜0.8-1.1 at 37° C., and induced with 250 μM isopropyl β-D-1-thiogalactopyranoside. Proteins were expressed overnight at 18° C. Cells were collected by centrifugation at ≥10,000 g for 20 min at 4° C. and pellets were stored at −80° C. until further processing.

For crude samples, pellets were lysed by sonication in 50 mM sodium phosphate, 150 mM NaCl, at pH 7.5. Lysed materials were centrifuged at 7,000 g for 20 minutes, then supernatants were centrifuged at 20,000 g for 1 minute, then supernatants were collected. Serial 1:10 dilutions of this crude lysate were prepared with phosphate buffered saline and then 100 μL of each dilution was mixed with 35 μL of 10 mg/mL xanthan gum oligosaccharides and absorbance was monitored at 235 nm.

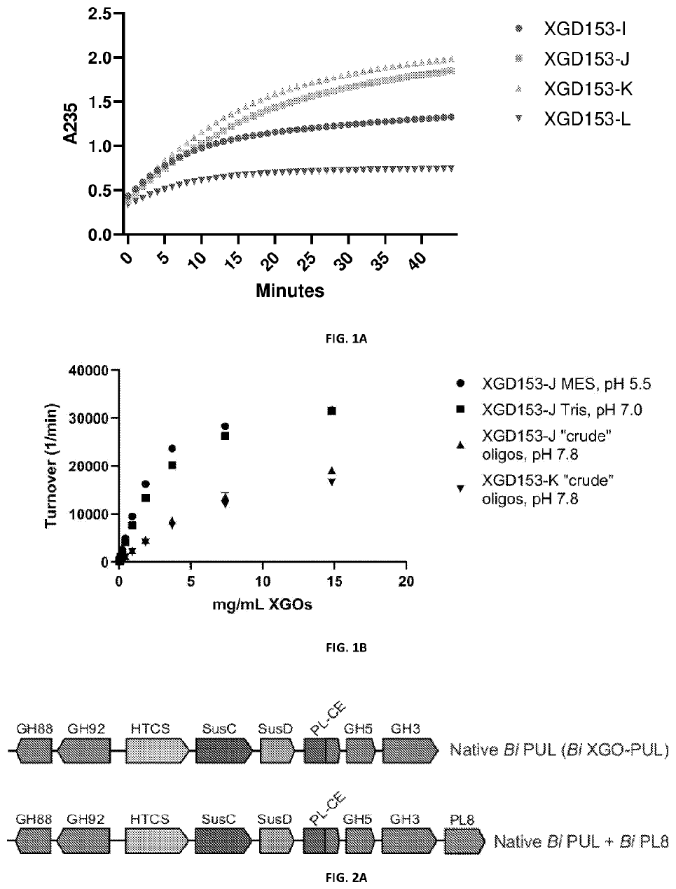

For full kinetics of XGD153-J and XGD153-K, pelleted E. coli cells that had produced each construct were lysed by sonication in 50 mM sodium phosphate, 150 mM NaCl, at pH 7.5. Lysed material was spun at 30,000 g at 4° C. for 1 hour to clarify, then supernatants were purified by standard immobilized metal affinity chromatography using HisPur Cobalt Resin (Thermo Scientific) and eluting proteins with 50 mM sodium phosphate, 300 mM sodium chloride, 100 mM imidazole, at pH ˜8. Protein concentrations were determined using absorbance at 280 nm and extinction coefficients. For kinetic assays, proteins were diluted using various buffers, as indicated in FIG. 1B, then mixed with serial dilutions of xanthan gum oligosaccharides (100 uL of xanthan gum oligosaccharides mixed with 35 uL of different enzyme dilutions) and absorbance monitored at 235 nm for reaction progress.

All XGD153 constructs (I, J, K, L) showed activity on xanthan gum oligosaccharides as demonstrated by increased A235 over time when mixed with this substrate (FIG. 1A). Further purification of XGD153-J and XGD153-K allowed full determination of their kinetics on pentameric xanthan gum oligosaccharides (FIG. 1B).

### Example 2

### PL8 Activity

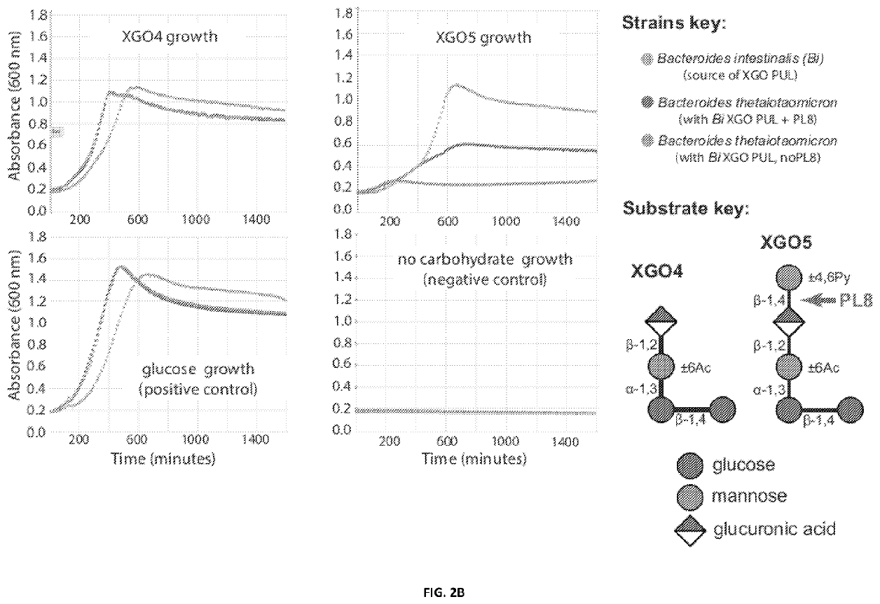

An eight gene polysaccharide utilization locus (PUL) is present in three genomes of a subset of Bacteroides intestinalis strains that are able to utilize xanthan gum oligosaccharides (XGOs) and that the genes in this PUL (FIG. 2A, herein referred to as the “Bi XGO-PUL”) are highly induced during growth on XGOs (See, e.g., PCT/US2021/050494, published as WO2022060859, incorporated herein by reference). Notably, none of the protein products encoded in this PUL were determined to catalyze the first step in XGO degradation, which is removal of the non-reducing end β1,4-linked mannose (with or without attached pyruvate). However, it was determined that a gene encoding a genomically unlinked polysaccharide lyase family 8 (PL8) enzyme (XGD153-WT) was determined to possess this activity. To test the concept that transfer of the Bi XGO-PUL, with or without the additional gene encoding the active PL8, confers the ability to utilize xanthan oligosaccharides, the 8-gene PUL was cloned into a conjugative vector (pNBU2, for plasmid non-replicating Bacteroides unit) that inserts into a neutral genome location. Delivery of this PUL-containing vector into the genome of Bacteroides thetaiotaomicron, a species that is unable to grow on XGOs conferred the ability to grow on XGO tetrasaccharides (XGO4) that lack terminal β1,4-linked mannose (FIG. 2B, top left panel). As expected, the Bi XGO-PUL alone was insufficient to confer growth on XGO5, the native repeat unit of xanthan gum that can be produced, for example, by xanthan gum hydrolysis using the R. UCG-13 GH5 endo-acting enzyme (FIG. 2B, top right panel, compare blue and orange curves). However, an engineered B. thetaiotaomicron strain that contained the Bi XGO-PUL plus the additional B. intestinalis PL8 gene recombined into the major operon of the Bi XGO-PUL downstream of the GH3 coding gene, and therefore under control of the native Bi XGO-PUL promoter, was capable of growing on XGO5 (FIG. 2B, top right panel), albeit not at the same rate and growth efficiency. Similar results were also obtained when the same two-step engineering process was performed with a different Bacteroide vulgatus strain (not shown).

It is understood that the foregoing detailed description and accompanying examples are merely illustrative and are not to be taken as limitations upon the scope of the disclosure, which is defined solely by the appended claims and their equivalents. Various changes and modifications to the disclosed embodiments will be apparent to those skilled in the art and may be made without departing from the spirit and scope thereof.

Numerous references, including patents and various publications, are cited and discussed in the description of this invention. The citation and discussion of such references is provided merely to clarify the description of the present invention and is not an admission that any reference is prior art to the invention described herein. All references cited and discussed in this specification are incorporated herein by reference in their entirety.

Sequences:

SEQ ID NO: 1-XGD153-I
MGSASGVDELVRIKQNYARMLVPSGKDPFGLLSILSSIQPETEISDQVVVELHQRYPFDLKK
VETYLSSFTEAGTWPDINYDDKKRSGWEPKIHAERILELVKLYNSDQTSYYRSSEVEAVIHK
ALNYWFTAKPVCLNWWYNQIGIPKTLGTVFILFEKQLTPVEKQNAITVMENAKFGMTGQN
KVWLAGNVMMRALLQNDYELVKMARDTIASEIVTGGTEGIKDDWCFHQHGAQQQFGNYG
LSFVSGMSFFSGLFSGTSLAFDDKQLSILSTLIDKGYRWVIWKGMMDVNALGRQLFHHAPV
HKALSLAFAASELGGGESDECMAVATALLRDNYPAPAVNVLTGHKHFWQSDYTIHRRPSW
MASIKMASDRIIGTEMMNGDNMKGYYMADGATYIYKDGKEYLDIFPLWDWRKLPGVTAF
EDNAPMPLIKSYQPRNKGTFVGAVSDEKQGMTVMELDRAGVKAHKAWVCTDDFILCLGA
GIQADSNLVVTTSIEQCHKNGELLSWENARWNVVSTKQSVKGKEQRYFHNNTGYIVWGNT
HEVVAETAERTGSWYDVMQMYHPEETHGEVTAIYLTHGVAPKQGTYQYLILPGMDKENV
AAFNFSDIRILRNDATVQAVYSEGNTTCWVAAYQPVQLTVSTDLILNIQTPGIYMIRKNESGR
YIINYADPTQQRNVAELELNHKKVRLSLLEG
SEQ ID NO: 2-XGD153-J
MGSGVDELVRIKQNYARMLVPSGKDPFGLLSILSSIQPETEISDQVVVELHQRYPFDLKKVET
YLSSFTEAGTWPDINYDDKKRSGWEPKIHAERILELVKLYNSDQTSYYRSSEVEAVIHKALN
YWFTAKPVCLNWWYNQIGIPKTLGTVFILFEKQLTPVEKQNAITVMENAKFGMTGQNKVW
LAGNVMMRALLQNDYELVKMARDTIASEIVTGGTEGIKDDWCFHQHGAQQQFGNYGLSF
VSGMSFFSGLFSGTSLAFDDKQLSILSTLIDKGYRWVIWKGMMDVNALGRQLFHHAPVHKA
LSLAFAASELGGGESDECMAVATALLRDNYPAPAVNVLTGHKHFWQSDYTIHRRPSWMAS
IKMASDRIIGTEMMNGDNMKGYYMADGATYIYKDGKEYLDIFPLWDWRKLPGVTAFEDN
APMPLIKSYQPRNKGTFVGAVSDEKQGMTVMELDRAGVKAHKAWVCTDDFILCLGAGIQA
DSNLVVTTSIEQCHKNGELLSWENARWNVVSTKQSVKGKEQRYFHNNTGYIVWGNTHEVV
AETAERTGSWYDVMQMYHPEETHGEVTAIYLTHGVAPKQGTYQYLILPGMDKENVAAFNF
SDIRILRNDATVQAVYSEGNTTCWVAAYQPVQLTVSTDLILNIQTPGIYMIRKNESGRYIINY
ADPTQQRNVAELELNHKKVRLSLLEGKEKGKTTSIVG
SEQ ID NO: 3-XGD153-K
MGSASGVDELVRIKQNYARMLVPSGKDPFGLLSILSSIQPETEISDQVVVELHQRYPFDLKK
VETYLSSFTEAGTWPDINYDDKKRSGWEPKIHAERILELVKLYNSDQTSYYRSSEVEAVIHK
ALNYWFTAKPVCLNWWYNQIGIPKTLGTVFILFEKQLTPVEKQNAITVMENAKFGMTGQN
KVWLAGNVMMRALLQNDYELVKMARDTIASEIVTGGTEGIKDDWCFHQHGAQQQFGNYG
LSFVSGMSFFSGLFSGTSLAFDDKQLSILSTLIDKGYRWVIWKGMMDVNALGRQLFHHAPV
HKALSLAFAASELGGGESDECMAVATALLRDNYPAPAVNVLTGHKHFWQSDYTIHRRPSW
MASIKMASDRIIGTEMMNGDNMKGYYMADGATYIYKDGKEYLDIFPLWDWRKLPGVTAF
EDNAPMPLIKSYQPRNKGTFVGAVSDEKQGMTVMELDRAGVKAHKAWVCTDDFILCLGA
GIQADSNLVVTTSIEQCHKNGELLSWENARWNVVSTKQSVKGKEQRYFHNNTGYIVWGNT
HEVVAETAERTGSWYDVMQMYHPEETHGEVTAIYLTHGVAPKQGTYQYLILPGMDKENV
AAFNFSDIRILRNDATVQAVYSEGNTTCWVAAYQPVQLTVSTDLILNIQTPGIYMIRKNESGR
YIINYADPTQQRNVAELELNHKKVRLSLLEGKEKGKTTSIVG
SEQ ID NO: 4-XGD153-L
MGSGVDELVRIKQNYARMLVPSGKDPFGLLSILSSIQPETEISDQVVVELHQRYPFDLKKVET
YLSSFTEAGTWPDINYDDKKRSGWEPKIHAERILELVKLYNSDQTSYYRSSEVEAVIHKALN
YWFTAKPVCLNWWYNQIGIPKTLGTVFILFEKQLTPVEKQNAITVMENAKFGMTGQNKVW
LAGNVMMRALLQNDYELVKMARDTIASEIVTGGTEGIKDDWCFHQHGAQQQFGNYGLSF
VSGMSFFSGLFSGTSLAFDDKQLSILSTLIDKGYRWVIWKGMMDVNALGRQLFHHAPVHKA
LSLAFAASELGGGESDECMAVATALLRDNYPAPAVNVLTGHKHFWQSDYTIHRRPSWMAS
IKMASDRIIGTEMMNGDNMKGYYMADGATYIYKDGKEYLDIFPLWDWRKLPGVTAFEDN
APMPLIKSYQPRNKGTFVGAVSDEKQGMTVMELDRAGVKAHKAWVCTDDFILCLGAGIQA
DSNLVVTTSIEQCHKNGELLSWENARWNVVSTKQSVKGKEQRYFHNNTGYIVWGNTHEVV
AETAERTGSWYDVMQMYHPEETHGEVTAIYLTHGVAPKQGTYQYLILPGMDKENVAAFNF
SDIRILRNDATVQAVYSEGNTTCWVAAYQPVQLTVSTDLILNIQTPGIYMIRKNESGRYIINY
ADPTQQRNVAELELNHKKVRLSLLEG
SEQ ID NO: 5-XGD153-WT (B. intestinalis PL8)
MRLLRNLIIISVLLVSVTVQASGVDELVRIKQNYARMLVPSGKDPFGLLSILSSIQPETEISDQ
VVVELHQRYPFDLKKVETYLSSFTEAGTWPDINYDDKKRSGWEPKIHAERILELVKLYNSD
QTSYYRSSEVEAVIHKALNYWFTAKPVCLNWWYNQIGIPKTLGTVFILFEKQLTPVEKQNAI
TVMENAKFGMTGQNKVWLAGNVMMRALLQNDYELVKMARDTIASEIVTGGTEGIKDDW
CFHQHGAQQQFGNYGLSFVSGMSFFSGLFSGTSLAFDDKQLSILSTLIDKGYRWVIWKGMM
DVNALGRQLFHHAPVHKALSLAFAASELGGGESDECMAVATALLRDNYPAPAVNVLTGHK
HFWQSDYTIHRRPSWMASIKMASDRIIGTEMMNGDNMKGYYMADGATYIYKDGKEYLDIF
PLWDWRKLPGVTAFEDNAPMPLIKSYQPRNKGTFVGAVSDEKQGMTVMELDRAGVKAHK
AWVCTDDFILCLGAGIQADSNLVVTTSIEQCHKNGELLSWENARWNVVSTKQSVKGKEQR
YFHNNTGYIVWGNTHEVVAETAERTGSWYDVMQMYHPEETHGEVTAIYLTHGVAPKQGT
YQYLILPGMDKENVAAFNFSDIRILRNDATVQAVYSEGNTTCWVAAYQPVQLTVSTDLILNI
QTPGIYMIRKNESGRYIINY ADPTQQRNVAELELNHKKVRLSLLEGKEKGKTTSIVG
SEQ ID NO: 6-Rucg13 GH5 domain
KIVKQGTDEMVVLRGVNVPSMDWGMAEHLFESMTMVYDSWGANLIRLPINPKYWKNGSV
WDEKNLTKEQYQKYIDDMVKAAQARGKYIILDCHRYVMPQQDDLDMWKELAVKYGNNS
AVLFGLLNEPHDIKPVGVEKPTTVEQWDVWYNGGQIIVGGEEVTAIGHQQLLNEIRKQGAN
NICIAGGLNWAFDISGFADGYNERPNGYRLIDTAEGHGVMYDSHAYPVKGAKTAWDTIIGP
VRRVAPVIIGEWGWDSSDKNISGGDCTSDIWMNQIMNWMDDTDNQYDGIPVNWTAWNLH
MSS
SEQ ID NO: 7-Truncated xanthanase
MEEAAADAQNAEINYNRSVPLEVKGNKIVKQGTDEMVVLRGVNVPSMDWGMAEHLFESM
TMVYDSWGANLIRLPINPKYWKNGSVWDEKNLTKEQYQKYIDDMVKAAQARGKYIILDC
HRYVMPQQDDLDMWKELAVKYGNNSAVLFGLLNEPHDIKPVGVEKPTTVEQWDVWYNG
GQIIVGGEEVTAIGHQQLLNEIRKQGANNICIAGGLNWAFDISGFADGYNERPNGYRLIDTAE
GHGVMYDSHAYPVKGAKTAWDTIIGPVRRVAPVIIGEWGWDSSDKNISGGDCTSDIWMNQI
MNWMDDTDNQYDGIPVNWTAWNLHMSSSPKMLYSWDYKTTAYNGTHIKNRLLSYNTAP
EKLDGVYSTDFSTDDVFRSYTAPSGKASIKYSDESGNVAITPAAANWYATLNFPFDWDLNGI
QTITMDISAATAGSVNIGLYGSDMEVWTKAVDVNTEVQTVTIGINELVKQGNPQTDGKLDA
ALSGIYFGAATADTGSITIDNVKIVKLATPVYTANTYPHKDMGEESYIDIDTTGFKKQTTAW
NSKFTGTTMQITDANVLNINGETTKTKCVTYTRDATDTEGCRAKFDLNTVPSMDAKYFTIDI
KGNGIAQKLTVSLSGLAYITVNMAEGDTDWHQYIYSLEGNVEYPEDITYVQISADTRTTAEF
YIDNIGFSNTKSERLIPYPEKTFVYDFATYNKNTTKYEAAISTESGSEGDTIVATKEEGGLGFD
SKALEVKYSRNGNTPSKAKVVYSPNDFFKGNVNDDERTANRATLKADMEYMTDFVFYGK
STSGKNEKINVGVIDTASAMTTYTDTKEFTLTTEWKQFRVPFDEFKILDGGSNLDCARVRGF
IFSSAENSGEGSFMIDNITHTSIKGDIEWGLP
SEQ ID NO: 8-Full-length Rucg13 GH5-30 enzyme
MERVIFMKKFLSLVTAIVMTVSLCIMPVYAQTYEEAAADAQNAEINYNRSVPLEVKGNKIV
KQGTDEMVVLRGVNVPSMDWGMAEHLFESMTMVYDSWGANLIRLPINPKYWKNGSVWD
EKNLTKEQYQKYIDDMVKAAQARGKYIILDCHRYVMPQQDDLDMWKELAVKYGNNSAV
LFGLLNEPHDIKPVGVEKPTTVEQWDVWYNGGQIIVGGEEVTAIGHQQLLNEIRKQGANNIC
IAGGLNWAFDISGFADGYNERPNGYRLIDTAEGHGVMYDSHAYPVKGAKTAWDTIIGPVRR
VAPVIIGEWGWDSSDKNISGGDCTSDIWMNQIMNWMDDTDNQYDGIPVNWTAWNLHMSS
SPKMLYSWDYKTTAYNGTHIKNRLLSYNTAPEKLDGVYSTDFSTDDVFRSYTAPSGKASIK
YSDESGNVAITPAAANWYATLNFPFDWDLNGIQTITMDISAATAGSVNIGLYGSDMEVWTK
AVDVNTEVQTVTIGINELVKQGNPQTDGKLDAALSGIYFGAATADTGSITIDNVKIVKLATP
VYTANTYPHKDMGEESYIDIDTTGFKKQTTAWNSKFTGTTMQITDANVLNINGETTKTKCV
TYTRDATDTEGCRAKFDLNTVPSMDAKYFTIDIKGNGIAQKLTVSLSGLAYITVNMAEGDT
DWHQYIYSLEGNVEYPEDITYVQISADTRTTAEFYIDNIGFSNTKPERLIPYPEKTFVYDFATY
NKNTTKYEAAISTESGSEGDTIVATKEEGGLGFDSKALEVKYSRNGNTPSKAKVVYSPNDFF
KGNVNDDERTANRATLKADMEYMTDFVFYGKSTSGKNEKINVGVIDTASAMTTYTDTKEF
TLTTEWKQFRVPFDEFKILDGGSNLDCARVRGFIFSSAENSGEGSFMIDNITHTSIKGDIEWGL
PTPTPEPTPTPLPDPVTVTTAEQLAAITSTEGNIILGADIDLGTTGFTTKSVTHLDLNGHTLTSS
GPFVVDPRHEITIVDTGSTKGAIINTGTTQTSYGIRGTTEAATINIDGAEIDAGGQAILINVAGR
KCNIKDAVINGGSYAINVGTNGGEINIDNALINNKADYKGYALYLQGGIAIIDDGTFGYNGT
TNTLLVARSSELTINGGTFTNPNSGRGAIVTDKQFVGTVTINGGVFENTNAGGYSILDSNEGY
QSIDAETSEIIASPVININDGTFKSAIGKTKSTNSSATEISIKGGQFAADPTVLYPNCIDTDIYSIT
KVAEGKYVVTKKGVEPTPEPTPEPVAKIVSSIEEINTLTASDDYVKLGADIDLGTSSIKTKCA
MRLDLNGHTLSGGGSTVIEAMYNLTVVDTGTTKGTIKNVNTSTSYGIKFAVKDAVLTIDGA
KVEAMSQAIMLSGTGSILHLKDSVINGNSYAVNLSNGIINIENTVINDDSEYKGYALSVANGT
AVINSGIFNYNGNMSSITFSGSSEITINGGTFKNSVSKRGAINTVKGFSGTLTINGGTFENTAE
NNGYSILDGDEATTETVPVINITGGTFKSTIGATKPANTTTVITISGGTYSFDPTSYVTDTETY
RVIDNGDGTYKVAPNSQVYSVTLNACGGSEVMVEDFKEENIPDNGIELPIPTKAGYKFDGW
YTEENNGSQVNGITKDNLSDIFRNEATVTLYAHWTLLNYTITYEGLNDATNTNPSNYTVETE
AITLAAPGTRKGYTFGGWYTDVEYQNKIEIIEQGTTGNKILYAKWDEIASGSITASFVSTGTIP
SDIVQGTINVTEKAYENDEVSFMVTLPKGYTLENVLCTADGENLNTITEENGSYTFIMPGKN
VTITVNVRPIQYTINLDLQEGTGTTTTIYGSVENLPVLPNDNPKKQGYNFKGWFDAPTKGTVI
TMDNLNTASNMLALFGNNTELTIYAQYTEVGNFVVIYSAVGADEETIPTDNTQYNIAETSIIK
IPNQEPKKLGYTFEGWKTGTDDTVYKYGTQNDTYTVPNDINGAITFIAQWSINEYEITYELN
GGINAENAPVSYTIETDTITLPVPTKDGYNFEGWYTDAAFENAVAAIAKGSVGDMVLYAKW
SEKDMAVYKINNYEKGNVSVRKRTDTDDSSSVVIVAFYKTLNNNSVLIKTSIAEIGAIEKGD
DISKTVEEPEDYSYAKVFMWNDLNGMMPRCNSPKMDK
SEQ ID NO: 9-Rucg13 PL8 (polysaccharide lyase 8 family protein)
MILLIHIKMGGMIMTDFNILRKRYSDVLCGRGYNGKKTADCILQSDERTEQRLVQLGGRIEK
AITSNEPGVINATLKGILDISISFSQNNSQFYHNKNIKNEIFNALNTLEKVYNDTTVPKGNWW
YWEIGIPLSINSIFTLMYDYTDKSQLKRYMAAEKHENDRIKLTGANRIWESVIFAVRGILLSD
NDSIKNAISGIQDVMVITDSGDGFYKDGSFIQHDNIPYNCGYGRSLIQELAPMLYIFKDTEFEN
KNTDIINTWIEKSYLPFIYNGRAMDMVRGREISRYYEQSDLACTHILSAMLILSEMPEFNELK
GTIKTQITDNFFEYASVFTAELAEHLQEDNNIKPKEIKPYFMAFNSMDRVVKHGNGYTIGLA
MHSERTAAYESINDENQNAHHTSDGMMYIYKKNEPKSDFFWQTIDLQRLPGTTVLRGSTVK
PNINAAGDFTGGCGIGENGVCTMKLISNENSLKANKSWFFFDKEVVCLGSCINSEEESEVETI
IENRLVTDNSRFTVHGNEESEGYIIKGAYLDGSHDVGYCFPEEQEVNIFREIRSGDWNNMSIK
SDGKSYKGRYLTMWIKHGRKVKDVSYEYIVIPKCHEEEINDYYRKSGIRIIENSDSIQCVKKN
GTTGVVFLKDKTHSAGGISCDRRCIVMTTQTCGTLELSISDITQKQDKIYIELDYSAQEIISKSE
RINIIQLVPYVCMEIDTCAARGEEQHIKFGGVKNV
SEQ ID NO: 10-Rucg13 GH94
MENLLVRRTNMKYGYFDDLNKEYVIETPRTPLPWINYLGTNGFFSLISNTSGGYCFYKDAK
HRRILRYRYNNIPADNGGRYFYINDNGDCWTPSYMPMKKELDFYECRHGMGYTKITGERN
GVRVEQTAFVPVDDNCEIHRIKVTNTSGEAKNINLFSFVEFCLWNAQDDMLNYQRNLNTGE
VEIDGSAIYHKTEYRERRNHYAFYSVNTEISGFDTDRDTFLGAFNGLDTPDRVINGKSGNSV
ASGWYPIASHQIDVSLDAGESREYIFVLGYIENEKDEKFESLNVINKTKAKEMIARYESSAQC
DAELDKLKLYWDNLLSVFTLESNDEKLNRMVNIWNPYQCMVTFNMSRSASYYESGIGRGM
GFRDSNQDLLGFVHQIPERARERIIDIASTQFEDGSAYHQYQPLTKQGNNEIGGGFNDDPLW
LILGTVAYIKETGDYGILDEQVPFDCDKNNTATLLEHLNRSFGHVTNNLGPHGLPLIGRADW
NDCLNLNCFSEIPDESFQTTGDDDGRVAESVLIAGMFVYIGREFARLYKTLNNDEMYKYISD
EVEKMTEAVLEYGYDGEWFIRAYDANGNKVGSDECDEGKIFIESNGFCVMAGIGKEDGRA
QKALDSVKKYLECEYGIVLNYPPYSGYRLELGEISSYPPGYKENAGEFCHNNPWVIIGETVM
GNGERAFELYKKIAPAYLEEISEIHKTEPYVYSQMIAGRDAVRAGEAKNSWLTGTAAWNYY
TVSQYLLGIRPDFDGLVIEPCISKDISEFKVTRKFRGKTYNILVKNTGEGTVKITADNGTVNG
TTVSSDAEICNVEVVM
SEQ ID NO: 11-Rucg13 GH38-8
MILIYNSDIMYNKYIKPKFIIWYKKEFQMSKNVHIISHSHWDREWYLPFEQHRMRLVELIDK
CMEVFEKDDSFKSFFLDGQTIVLDDYLEIRPENKEKLIKYTKEGKFIIGPWYILQDEFYTSGEA
NIRNLLVGMKEAEKYGAMCKMGYFPDAFGNAGQMPQLLKQAGMDTVTFGRGVRPVGFD
NEVQENGNYESPYSEMMWESPDGTKIFGILFANWYNNGNEVPTDKKIAKEYWDDRLKKVA
TFASTDEYLLMNGCDHQPVQADLGKAIEVASELYPDINFKHSNFPEYIKAIKEKVPNDLAVV
KGELTSQDTDGWSTLMNCASSHIYLKQMNRKCESALENGAEPVRVLSSVLGQNYPSDELEY
SWKKLMQNHPHDSICCCSVDEVQDEMATRFNKSKQVADYLVSEGKRYIADKINTKEYEKY
KNALPFVVENTAGRERTSVVSVEIDVTRKSGWLKKCAYDLDEINVPNYKLIDSDGNSIPFKIE
DLGVKFGYDLPKDKFRQPYMARRVRVTFEAENISAVGYKTYALVEGDTEKVTDTLVSSEN
CMENDAIRVEINKNGSLNVTDKASGRTYKGVAYYEETGDLGNEYMYKMPEGSKAITTQDT
VAKIELAEDEPYRAMYKITNTITVPKSGDDNFEDEKSHMVFFKERVGGRSNDTVEMKIETFV
SLDKNGKGVKIKTRFDNEVKDHRVRIMVPTGINSDVHKADSVFEVVTRNNRHNAGWNNPS
ACEHEQGFVSIDDGEKGIAVANIGLYEYEMLPDLDNTIAVTILRAVGEMGDWGVFPTPKAQ
CLGISETEIEIVPFKGDLISSGAYEECYQFRTDIITADTDCHDGVMPLDYSMINWQGNGLILTG
IKQKGNGEDIIIRWVNVSDKTTTLTIQKSDVIDNLYISNIIEKKIKKIDSNNNYFNIEVKPYEIM
TVGIAK
SEQ ID NO: 12-Rucg13 GH38-30
MERKNIKCHFISNTHWDREWKFSAQRTRHMLVTAIDMLLDIFEKEPDYKHFHLDSQTLPIQD
YLEINPEKKEILKKYISEGKLAVGPWFCLPDEFCVGGESLIRNLLLGHKIANEFGKVSKTGYS
PFGWGQISQMPQLYHGFGIDFASFYRGLNTYMAPKSEFYWEGADGTTIYASRLGQRPRYNM
WYIMQRPVFYGKRDGDNRRVSWGAGDGIFRFADPARCEYEYQYSHRKYEYHDEYIAEKTE
QALSEQDDEWTTPNRFWSNGHDSSIPDMRESRLIKDANAVYEGVDVFHSTVYDFEQSVIRD
FDKNSPVLKGEMRYPFTKGSVSALFGWVLSARIKVKQENFETERLLTSYAEPMAVFASVCG
AVYPQAFINKAYNYMLQNHGHDSIGACGRDVVYKDVEYRFRQSREIATCVLERALMDLSG
DIDFAGWDKNDMALVMFNPAPFKRSLTVPCELEIPLEWECDSFEIVDAEGNVCPHQNISSINP
MYQIVQDLADAVDVLPVSRHTIRIFVKDIPSMGYKTLKVVPKYHTRATTPVNMLCGINTME
NEYLKVKINSNGTLKVTEKETGREYDNIGYFKDTGENGSPWEHKTPELDEEYTTVNERAIVS
LVYSGELETKYRIVLNWAIPENIVDGGKKRSSRLAPYRIETLVTLRKGARWVEFETKINNNV
PNHYLQAAFPTDVDAEFVYAQGQFDVVKRPIAKPDYSKYDEIPMTEQPMNSFVDICNENEG
AAILNTGLKAYESDDDYNHTVYLSLLRCFELRIYVTPEEQNYSRIENGSQSFGEHTFRYAFM
PHKGDWEDAQVWKAAEDFNMEILIGQTAPTEHGKNPLEKSFIELENENLHISAVKRSEDGL
GCVVRLFNPSSETVKNRIRFNGGIAEISDKQSPIERQVHSFELPCTENRKWASVKKVTLEELS
ETELSVDTNGWCDVEVTPKQIYTLKYE
SEQ ID NO: 13-Rucg13 GH88
VNIDKAITYAESIVRKSLNYFYDCFPTEQSENLVFKKFENVSWTTGFYEGILWLMYELTGDK
AFYNSAKHHSEMFHKRLVDRVELEHHDMGFLFTLSSVADYRITGDEQAKQDGIEAAEWLL
KRYQPKGKFIQAWDAMDDSQSYRFIVDCMLNIPLLFWASEVTGYKKYYDAAYNHMQTSIA
NIIRPDASSYHTFFFDPVTNKPLRGETHQGFSDDSSWARGQSWAVYGLALCYHYTKEKSILP
LFERVTHYFIDHLPEDSVPYWDLIFSDGSDEPRDTSAAVVAVCGILEMEKYYHNQEFLDAAE
KMMTSLSEKYTTVDYPQSNGIIKDGMYSRKHGHEPECTSWGDYFYLEALMRMKKSDWKIY
W
SEQ ID NO: 14-Rucg13 CE (carbohydrate esterase)
MKKIISLMLAVTMICASIGLTAFAATTTTVEAEADGVSAYTLPSSDKSNSKILKNTVSSKESV
TYYIQANNTPRATMFKLAQVNTGDKINVDINFTYLDTATMELEYCLFVSDSEITLTSHSQDL
VKEELEKHTDESNIKNWSTNKSNMKYSLPNGITASKDGFVYLYIGCGDLSEDKTQVTKKIQ
WSIDSFDVNIDSDGGGETEPDTTPTPTTTINPDVTPTPTPTASPTPTPTLEPELTLNAVYSSNM
VLQRKEPITITGTGKSGNTVSVNFNGADEQTTIEHGLWEITLPAMEAVKSATMTVSSGDNMI
TLDNVAVGDVIFCTGQSNMFNRLETFPTLMNEELSEAYEDVRYMNSFDEISEWKVATMENS
KQFSALGFLIGKRMIKKDSDVPIGLISSSLGGSSIMQWIPTYSVNWDSQAKRMMAGASSKGG
LYTQRLLPLKNLKASAVVWYQGEANTTFESGTVYEQALTSLINNWRKTFNDEDLPFVVIQL
PTANFAKIYSTIRIGTGVRAGQWNVSQRMDNVKTVVSNDTGTTNNVHPNDKGPIADRAVA
YIEDFINNTQSNVESPSFDYMERSGDKLILHFKNTYGSLSTDDGGVPLGFELKDDDGIYKDVT
PTINGDTIEIDVTDITNPQVKYAWSDTPGIAKDLVEAQTDTPAVINTFNAAGRPIAPFMTDLT
EKYASKAVNKELSTTEFYNYAPYISKVEQSGDDIVISAYDTDGVVSKVEVYIDEGEIKAGDA
KQRDDGKWVFTPDVTSGVHSVYAIATDNDNINSLTCVDYTTYNIIRPTRYDYVKGYTESPSS
VEYNNGDDMLAKATNDVNGTTTTVTSAIPTGETTKSLKLSATGNKATANATIPISKADNPQ
KTLTIEYDTMFESADDAIGASRGMYAKTKEGNELWLTYFTASSLRTAITNTGGNWCYEQA
MSIKNNQWHHIKLELHPNTGIFSIWLDGTMLQDNVSFVKEGSSFDTCKGAFDTLKEGITDLR
FYHTASNNIENATYIDNVKVTEVSYSEEEIIPPAKIQEATPQISIDYINETLTGFESQEPYTIKVG
EGNAKDITLGEGVTTISLDDEKIGYAGKLLSIEIVKKARNTETYTDSDVQQLTVKARPKAPTT
VQGVNATEIGGKGKLTGMNGMQYKLKRTDEWSSTQLVDTVEVDAGEYNVRKAATDTDFA
SEKTTITVETFIAEKEMTPEIAIDYTTEELINFVEDGTYTINGLDVTLTDNKLSLANYITNEQIT
LSIVKKGNNVTTVASEAQTLIVKARPAAPTKSEIIVTQPSVIGGKGTIAGIADTMEYSTNNGIN
WTTGDGDDIGDIEPGTTYKIRYKAVSADEEAERQFKSAEYSVTIIAYDAMPETQPTISINYVN
EKLTGFTEGCDYIIKIDDGVATDKDNVTEDIDIDNTYFGHTLKIVKKDDGIKTSNSEAFELSIP
KRSSAPNVAAVEEQTYQGNDGKITGVDTTMEYKSLSEPTFTWMQCVGTEITNLAPGSYIVR
VAAVADESFASEVMSVTINAAAKDEPTEPTVNITYDDKNGNVNAIFTNITEEGMVYVAEYN
ENGTLLSIKSDEISDSVIIPFTCVNKSKVKVFIWKNDMKPLFNKVFTLN
SEQ ID NO: 15-Rucg13 altCE (carbohydrate esterase)
MFNKKFNLLKEATEYGFMPYMETYILDGKKRPIVVIFPGGGYGMVSEREAERIAMAYNAAG
FHAAVVYYCVEPHTHPLPIQNAANAVAMLRENAEKWNIDTDKVIVCGFSAGGHLAASLSA
LWNDSEIFSEREIELAMHKPNAQILSYPVITSGEFAHKDSFKNLTGTDDESNHLWSSLSLERRI
TDIIPPTFLWHTYEDICVPVENTLMYAAGLRRVGVPFELHIFEKGEHGLSRVSDELIWSKRKF
EREYPWLSLSVDWLNQLF
SEQ ID NO: 16-B. intestinalis SusD
MKKRHIIGSFLLGLLLTVNTGCEDFLDQKDTSGINENSLFLKPEDGYSLVTGVYSTFHFSVDY
MLKGIWFTANFPTQDFHNDGSDTFWNTYEVPTDFDALNTFWVGNYIGISRANAAIPILQRM
KDNGVLSEKEANTLIGECYFLRGVFYYYLAVDFGGVPLELETVKDEGLHPRNSQDEVFASV
VSDMNIAAGLLPWKAEQGSADRGRATREAALAYQGDALMWLKQYKEAVEVFNQLDSKC
QLEENFLNIHEIANRNGKESIFEVQFTEYGSMNWGAFGVNNHWISSFGMPVAISGFAYAYAD
KKMYDSFENGDLRRHATVIGPGDEHPSPLIDLQDYPKLKDFATKGNGNIPASFYQDEEGNV
LNTCGTVENPWLDGTRSGYYGVKYWRNPEVCGTRGAGWFMSPDNIMMMRYAQVLLSKA
ECLYRLNDSNGAMAIVQKVRDRAFGKLQNSAVEVPAPANTDVLKVIMDEYRHELTGETSL
WFLLRRTGEHANYIKEKYGITIPTGKDLMPIPQTQIGLNQNLKQNPGY
SEQ ID NO: 17-B. intestinalis SusC
MKTKFIATFFLLICGSVMFAQTRTVKGKVVDKANEPLIGVAVNIKNTSQGSITDFEGNYSIQV
NTENAVLVFSYIGYDKQEIKVGARNVIDVVMHEASIALDQVVVVGYGTSKRGDVTGSISSID
AAEIKKVPVVNVGQALQGRMSGVQVTNNDGTPGAGVQVLIRGVGSFGDNSPLYVVDGYPG
ASISNLNPSDIQSIDVLKDASAAAIYGNRAANGVVIITTKRGNADKMQLSVDATVSVQFKPS
TFDVLNAQDFASLATEISKKENAPVLDAWANPSGLRTIDWQDLMYRAGLKQNYNLSLRGG
SEKVQTSISLGLTNQEGVVRFSDYKRYNIALTQDYKPLKWLKSSTSLRYAYTDNKTVFGSG
QGGVGRLAKLIPTMTGNPLTDEVENANGVFGFYDKNANAVRDNENVYARSKSNDQKNISH
NLIANTSLEINPFKGLVFKTNFGISYGASSGYDFNPYDDRVPTTRLATYRQYASNSFEYLWE
NTLNYSNTFGKHSIDVLGGVSIQENTARNMSVYGEGLSSDGLRNLGSLQTMRDISGNQQTW
SLASQFARLTYKFAERYILTGTVRRDGSSRFMRGNRWGVFPSVSAAWRIKEESFLKDVDFIS
NLKLRASYGEAGNQNIGLFQYQSSYTTGKRSSNYGYVFGQDKTYIDGMVQAFLPNPNLKW
ETSKQTDIGIDLGFFNNKLMLTADYYIKKSSDFLLEIQMPAQTGFTKATRNVGSVKNNGFEF
SVDYRDNSHDFKYGVNVNLTTVKNKIERLSPGKDAVANLQSLGFPTTGNTSWAVFSMSKV
GGSIGEFYGFQTDGIIQNQAEIDALNANAHRLNQDDNVWYIASGTAPGDRKFIDQNGDGVIT
DADRVSLGSPLPKFYGGINLSGEYKGFDFNLFFNYSVGNKILNFVKRNLISMGGEGSIGLQN
VGKEFYDNRWTETNPTNKYPRAVWSDVSGNSRVSDAFVEDGSYLRLKNIEVGYTLPANILK
KASISKLRIFASVQNLFTITGYSGMDPEIGQSMSSSTGVAGGVTASGVDVGIYPYSRFFTMGF
NLEF
SEQ ID NO: 18-B. intestinalis GH3
MKTFILSFLIYAGCSLPLTAQQIKPAIPSDPEIEAKINKLLQKLTLEEKIGQMCEITIDVITDFSD
KENGFRLSESMLDTVIGKYKVGSILNTPFSIAQEKEVWADLITRIQKKSMEEIGIPCIYGVDQI
HGTTYTRGGTFFPQSINMAAAFNRQLTRRGAEISAYETKACCIPWNYAPVMDLGRDPRWPR
MWESYGEDCYVNAEMGVQAVKGLQGENPNHIGENNVAACIKHFMGYGVPVSGKDRTPSSI
SRTDLREKHFAPFLASIQAGALSLMVNSGVDNGVPFHANKELLTGWLKEELNWDGMIVTD
WADINNLCLRDHIAETKKEAIQIAINAGIDMSMVPYEVSFCTYLKELVEEGKVSMARIDDAV
SRVLRLKYRLGLFDNPYWDIRKYDQFASPEFASVALQAAEESEVLLKNEDDILPLAKGKKIL
LTGPNANSMRCLNGGWSYSWQGDKADECAQAYNTIYEAFCNEYGKESVIYEPGVTYKTSA
DALWWEENTPRIAQAVSAAEKADVIIACIGENSYCETPGNLTDLNLSTNQKDLVKALAATG
KPIILVLNEGRPRIIHDIVPLAKAVVHIMLLGNYGADALVNLVSGKANFSGKLPFTYPHLINSL
ATYDYKPCENMGQMGGNYNYDAVMDVQWPFGFGLSYTTYSYSNLKVNRTSFDADNELVF
TVDVTNTGKMAGKESVLLYSRDLVASITPDNIRLRNFEKVDLQPGETKTVTMKLKGSDLAF
VGADGKWRLEKGAFRMTCGTQKLEVHCTTTKIWQTPNISKSGI
SEQ ID NO: 19-B. intestinalis PL2
MKNTVLPLILFLCMLCLGSHLYAGHSMHPLNQISYVKKKIKEQQEPYFTAYRQLMHYADSI
QEVSQNALVDFAVPGFYDKPEEHRANSLALQRDAFAAYCSALAYQLSGEERYGQKACYFL
NAWSSTNKKYSEHDGVLVMSYSGSALLMAAELMMDTPIWNPQDKDAFKTWVSQVYQKA
VNEIRVHKNNWADWGRFGSLLAASLLDDKEEVARNVQLIKSDLFVKIAEDGHMPEEVVRG
NNGIWYTYFSLAPMTAACWLVYNLTGENLFVWEHNDASLKKALDYMFYFHQHPSEWKW
DTRPNLGAHETWPDNLLEAMAGIYNDASYLQYVESSRPHIYPLHHFAWSFPTLMPVSLKGY
DLTDNNTWANYNRYEVANKTVKKPVAIFMGNSITEGWNRSHPDFFTQNGYVGRGISGQVT
AQMLARFRADVLDLKPQVVCILAGTNDIAQNCMYMSVENIAGNIFSMAELAKANGIKVVIC
SVLPATRYSWRPTVQNPAGQIIQLNKLLQKYAQKNKIPYVDFHSMMKDEQNGLPQKYSKD
GVHPTKEGFSMMEPIIKEAIDKLLK
SEQ ID NO: 20-B. intestinalis GH5
MKNIYYILILCCLCLFSCDSHPDTKSSLPFGVNLAGAEFFHKKMDGVGQFGIDYHYPTTREF
DYWKSKGLTLIRLPFKWERIQRELYGELNREEIDYIKYLLDEAGARDMKILIDMHNYGRRK
DNGKDRIIGDSVSIDHFASVWKQIAGELKEHSALYGYGLINEPHDMLDSVPWFKIAQAAIEE
SRKVDLKTAIVVGGNHWSSAARWQEISDDLKHLHDPSDNLIFEGHCYFDEDGSGIYRRSYD
EEKAYPTIGIDRTRPFVEWLKTNNLRGFIGEYGVPGDDERWLVCLDNFLDYLSKENINGTY
WAAGAQWNKYILSIHPDDNYQTDKIQLGVLTKYLETKN
SEQ ID NO: 21-B. intestinalis GH88
MRKQLSLLLVSISLGWVGCAPDKQADTIHLDRQLEYCDAQIRRTLSEADQDSCLMPRSMEA
NQTNWNMSNIYDWTSGFWPGILWYDYEATGDEEIKAQAIRYTECLLPLVTPAHGADHDIGF
QIFCSFGNAYRITGNEEYKTVILKGAQKLAKLYNPKVGTILSWPGMVKRMGWPHNTIMDN
MMNLEILFWAARNGGGQELYDIAVKHAQTTMKYSFREDGGNYHVAVYDTIDGHFIKGVTN
QGYGDSSLWARGQAWAIYGYMMVYRETQDKTFLRFAEKVTELYLENLPEDYIPYWDFDAP
DMIKQPKDASAAAITASALIELSELEDTPSLASRYLNAATRMLGELSSERYQCRDIKPAFLMH
STGNQPGGYEIDASINYADYYYLQALLKYKKAMGL
SEQ ID NO: 22-B. intestinalis GH92
MKTRTLGICLFLLMNVSFIKGQSLADKVDMWMGTYGAGHCVVGPQLPHGSVNPSPQTAYG
GHAGYVPDQPIRGFGQLHVSGIGWGRYGQIFLSPQVGFNPGETDHDSPKQGEEATPYYYKV
MLSRYDIQVEISPTHHCVAYRFTFPETDQGNILLDIAHNIPQHIVPEVKGLFHGGEINYNPEQQ
TLTGWGEYSGGFGSTDAYKVYFAMKTDTPLKEVKITDQGDKALYACLALNKNPGVVHLN
VGISLKSIENASLFLSEEIADNSFNTVKENAKAIWDNTLSSIKIKSENEAEERLFYTTLYHSFV
MPRDRTGDNPHWDSESAHMDDHYCVWDTWRTKYPLMVLLRESYVAQTINSFIDRFAHNG
VCNPTFTSSLDWTSKQGGDDVDNIIADAIVKNVKGFDYEKAYALMKWNAYHARSKDYLRL
GWEPETGGIMSCSAGIEYAYNDFCTSEIAGIMHDENTQKELYERSGNWSQLFNPLQESHTYK
GFIVPRKANGEWVAIDPAKAYGSWVEYFYEGNSWTYTLFVPHQFDRLIEYCGGKANMIKRL
SYGFENNLISLNNEPGFLSPFIFTHCGRPDLTARYVSQIRKDNFSLLKGYSDNEDSGAMGSW
YIFTSIGLFPNAGQDFYYLLPPAFTDVELTMENGKKISIKVLKDTPDACYIKSVSINGKVLDK
GWIYHREIAEGATLVYELTNKENAWHINE
SEQ ID NO: 23-Rucg13 GIK Glucokinase A
MKYYIGIDLGGTNIAAGIVDKTGKIIAKDSVPTLNTRPIEAIMLDMTKLCKTLLDKSQMDINK
IEAVGIGCPGTVDNKNGIISYSNNIPMKNVPMRKFMEKQLNISVNLENDANAAALGEYTAN
GHNASSYILITLGTGIGGGAVINSKIYRGFNGVGIEPGHMTLINGGERCTCGKHGCWETYGS
VTALINQTKLKMTDNPDSLMHKISGKFGEVNGRVAFEAAKAGDKAGLEVVEKYTEYVADG
ITSVINIFEPEILVIGGGISKEGEYLLNPIRKFVEINEFNKYRPKTKIEIASLNNDAGIIGAALSAN
R
SEQ ID NO: 24-Rucg13 CBM1 1
MKKLVSLIIAMSIFFSINCAIFATNVSYMADFESADAKFGNSTTYSGTKNTAGDYSDFVKPE
WVADGGKENSTGLRITYKAATWYAGEVFFPIPVAWQNGADAEYLNFDYNGKGIVNISLST
GSAATDTLTKGTKYSYKLNADTNGEWQSISIPLSEFKNNGNPVTIANIGCVTFQAGENGGLS
NSASETKAMTAAELEAKARNGSIIFDNMELSNVGENVLNPNATPEPTEKPDNTTRTIDFDTY
TLSHKQTWAGFNNNDKTYSDSIKSEITENGKEGCALELTYKAATWYAGEIFMSIPKEWAINK
NSECLEFDAKGQGKIKISLETGEVVNGIRYGHTVTINTNDEWQKISVPLSEFVNNGNEVPLTD
VVGMAFSAAESGNLDNNAEETKMMSADELEEKAVTGCVVIDNITLAEQDTTSPTAAPEATT
QPTEISYVADFETADTKFASGKTWGGFKNKSNDYQDYIKAKWLQDGGVDGSTAFCVYYQS
ATYYAGEIFVPAPAVWTNNGAKGAEYLNFDYKGKGAVKISFSTGNTVDGTLTSGTRYTRRF
ELDSHGDWAKISVPLSEFVNGENIVNMTEIGTVTFQAAENANLDNNSDDTKAMSADELKEI
ARTGEIIFDNMTLSETEGKTTLFSSVKVTAEIDGKEITNLTNGDIKIKAIASDIEKDTNMVMIV
AVYKENGVIDTVRMAGQKIIGDGELMLDLNVTDAEHQTMKVFIFDDFTNLHPIINVTNFL
SEQ ID NO: 25-Rucg13 Glk glucokinase B
BMPTIRFVYTYSLLWWAERLCGKMYYIGIDLGGTNIAAGIVTEEGKIVVKDSVPTLSERPTD
EIVTDMANLSKKLVQSIGIEMNEIKGIGIGCPGTIDFETGEIVYSNNIKINHYPLADKFKEHIPL
PVKVDNDANCAALGEYKINRHCASVFALVTLGTGVGGGVIINGKVFRGFNGAAGELGHMTI
VSGGKMCTCGKEGCLESYASATALISQTKDALETHKDTIMHGIVKKEGKISGRTAFEAAKQ
GDEVAKKVVSNYERYLADGIVSIENIFQPEIIAIGGGISKEGDYLIEPIREYVYNTGFNKHMTK
TKIVAAQLFNDAGIIGAAMLAI
SEQ ID NO: 26-Rucg13 HK histidine kinase
MSEKFNNMSFRTKLLLSYIAVIILCIIIFGLTVFSSISRRFENEITDNNAQITGLAVNNMTNTMN
NIEQILYSVQANSTIEKMLTASNPPSPYEEIAAIEQELSKIDPLKATVSRLSLYIENRTSYPSPFD
SNVTASVYSKNEVWYKNTKELNGSTYWCVMDSSDANGLLCVARAFIDTRTHKILGIIRADV
NLSQFTNDIAHISMNNTGKLFLVYENHIINTWNDSYINNFVNENEFFKAISADSDKPQLVQIN
KEKHIINHSRLKDSSLILVRASKLDDFNSDIHIIEKSMITTGIIALLVALIFIFIFTRWLTAPITKLI
KHMERFENNYERIPIEITSHDEMGKLGESYNSMLNTIDSLITDVEDLYKKQKIFELKALQAQI
NPHFLYNTLDSIHWMARAHHAPDISKMVSALGTFFRHSLNKGNEYTTIENELNQISSYVSIQ
KIRFEDKFDVVYDIDENLLHCTIVKLTIQPLVENSIIHGFDEIEEGGMITIRIYPEDDYIFIDVIDN
GSGADTNELNKAITHELDYNEPIEKYGLTNVNLRIQLYFDKTCGLSFKTNETGGVTATIKIKR
KEPEYKTIDL
SEQ ID NO: 27-Rucg13 Pgm phosphoglucomutase
MQCRGGNVMNFNIPDLGIIDGSSGFRNLPSTTDGRFTSGEDGVKHIVCTGDGKVEFVAFENQ
TLAYVNSALGYGAYYPLHPVNRNGKIKAVLMDLDGTSVRSEEFWIWIIEKTTASMLDDESF
KLEESDIPFVSGHSVSEHLQYCIDKYCPGESLDKARNFYFDHVNREMKEIMEGRGRKNAFVP
QEGLKEFLLALKAKGIKIGLVTSGLYEKAMPEILSAFRALDMGEPTDFYDAIISAGYPLRKGS
VGTLGELSPKPHPWLYAETCAVGLGVGFDERGSVIAIEDSGAGVCSARIAGYTTIGLAGGNI
KESGTMPMCSRYCNNLAEILDYIEEEA
SEQ ID NO: 28-Rucg13 ManA M6P Isomerase
MFFSVLHMAIINIKGVKIVSELYPVRLIPVFKDYLWGGTKLKTVFNKKSELNILAESWELSAN
KDGQSIIANGKYQGYGLKEYIDIVGKEIVGTKGLALDDFPILIKFIDAKKNLSVQVHPDDEYA
TCHDGANAKTEMWYILDCNVGAYLYYGFKKDITKQEYQDAIRSNTITDVLNKVPVHKGDV
FFIPAGTVHAIGAGILICEIQQNSNTTYRVYDYDRRDKDGNKRELHIREALESSNLKKSTYSN
SVLDGDDIILTQCDYFTVRRLKVQNRVQLRIDKTSFHSLIITDGSGELYMGGEILKLNKGDSIF
IPAQNNEYTVSGPCEIILSFL
SEQ ID NO: 29-Rucg13 RR response regulator
MNVKLLICDDEKIIREGLASLDWNTRGIEVVGTAKNGEVAFELFQKMLPDIVISDIKMPTKD
GIWLSEQIHKISPNTKIIFLTGYNDFEYAQSAINNGVCQYLLKPIDEFELYEIVDKLTKEIHLEQ
QKAEKEIELRKTLRNSRYFLLNYLFNRAQYGILDFELFEISKKAAAMTTFVIRLDTDSTNYG
MNFMIFEALIEHLPKTINFIPFFSNSDLVFICCFNEPEGESEQKLFSCCENLGDFIDTEFNVNYNI
GIGIFTSEISELEASYTSALQALDYSDRLGQGNIIYINDIEPKSQLSAYQSKLIETYIKALKNND
EKQSKKSVKELFDVMERSDMNLYNQQRRCMSLILSISDALYDIDCDPTILFKNTDAWSLIRK
TQSPAELKTFVENITDVVISYIESVQKQKAANIITQVKALVEKNYARDASLETVASQVFISPC
YLSVIFKKETNITFKNYLIQTRIEKAKELLEKTDLKIYDIAEKVGYNNTRYFSELFQRICGKTP
SQYRASHNPSMPQDI
SEQ ID NO: 30-Rucg13 TR transcriptional regulator
MSDKKPLYKQIMDKLKERIKSGDFEYDAPFVTEDRITKEYGVSRITAIRALEELEHDGLINRK
RGSGSFVSKNAMSILGKDKEDNAAVTIHKKNRDISLVALVMPFDIKLGNMFKCFDGINSVLN
KENCFVSIYNANRSVENEEKILRSLLEQGIDGVICYPVRGGRNFEVYNQFLVKKIPLVLIDNYI
ENMPMSYIVSDNSGGGKALCEYALEHGHKKIGFFCRGRVNETISIRDRYMGYAAALEEKGL
GVNLDYVYANIDDKYEMLTEEERQQYGNVENYLKTIVNRMHEQGISCVLCQNDWVAIQVY
NCCKALDISVPNEMCIMGFDNISELDEMDGGNKIITVEQNFFELGVKAGETVLREINGEMPGI
KYIVPVKIAVRN
SEQ ID NO: 31-Rucg13 XoPP transporter A
MLVVLGASFTSESAISEFGFHAIPKEWSLDAYRYIITSKETILRAYGVTIFVTIVGTLMSTLVV
ALYAYPLSRKDFKYRKLFTFIAFFTMLFSGGTVAGYMVTTGILNLKNSIWVLIFPYVMNAW
HVIVMRSFYSMSIPTAIIEAAKIDGANEYQIYFKIVLHISLPGLATIALFATLTYWNDWWLPLL
YITEPQKYNLQYLLQSMISNIQNLTENSAQMGSANLLANVPKEGARMALCIIATLPILFVYPF
FQKYFIQGLTVGSVKE
SEQ ID NO: 32-Rucg13 XoPP transporter B
MLSMCIPGLIFFILFNYLPMFGIIIAFKQYRYDLGIWASPWNGLKNFEFMFSSPDAWVITRNTI
AYNLLFIFGGLVFNVAMAIGLSELRNKAVSKLCQTVVIMPHFLSYVIVSFLVLAFLHVENGLI
NRSLIPALGLEGVDWYSNPKYWPWILVIVNFWKTTGYGSVVYLAGIAGIDTSLYEAAKVDG
ASRWQQIRYITLPALVPLMVVLTILNVGKIFNSDFGLFYQVPLNTGALYPATNVISTYVYNM
LMSAGTGSVGMASAAAFYQSIVGFILVMTTNFIVKKISPENALF
SEQ ID NO: 33-Rucg13 XoPP transporter C
MIMGKDETSEPLSKKKGDKIMRKKIAALLAMLMLGGVLTGCGGGNKVATGGEDPNVVPED
TYEINWYMQGMPQEDVASVEAAVNDYLKDKINATLKMHRLESNQYSKQLNTMIAAGEYF
DIAWTTPGVLTYTANARNGAWLALDDYIDTYIPKTIEQLGEIADNARVDGKLYAIPTYKEM
ADSRGWTYRKDIAEKYNINMDNIKTFDELLPVLKMIKENEPNMQYPIDWGSDRTPEALMKY
EEIAGTAVIFYDTDKYDGKVVNLVETPEYLEACKWDNKLYNEGLVKKDIMTATDFEQRLK
DGKTFCYVDFLKPGKAKETSAKFDFELDQSTVSDIWQDNGAGTGSMLAVSRTSKNPERVLR
FLELLNTDATLSNLINYGIEGKHYTKIDDNTITIPDDTSYTLQGYQWMQGNVFLNYLTEGESP
DKVEALKAFNAEAKKPIDYGFKFDNTAVEAEIAACQTVKSEYRKQVIMGSMDPEPIMKEYA
AKLKAAGIDKIIEEAQKQYDEFLANKNKQ
SEQ ID NO: 34-Rucg13 XoPP transporter D
MKKLLILFLLASVMLSMCSGCTVEKTVESAQAVTVLKVIKPNYISDFTQNIAEFNEANPDIQV
KFIDAPTSTEKRHQLYVSALSGKDSSIDIYWINDEWTKEFVEQKYIKALDGEILLDNSRYIIDA
QERFSVNDSFYAMPVGMDTDVIFYRSDKIHNVPETWDGIINLCRNSDFGLPIKLGLTTSDIQD
MMYNIIEIKEAIGISYAETLNLYKEFIEEYKDIENYTDTIAAFKIGSAAMLMGNSSLWKKLNG
DTSAVKGNIMVASLPNKNQFVRSYALAINSNSKNQEAAIRFLDFMNGKEQQRRLSRDTSLIP
IIRELYDDEMILDANPHVKGIKQSVQNSSSFATVSINGENLKKLEEALIKFFNNEETSMNTGKI
FEDLMQ
SEQ ID NO: 35-B intestinalis HTCS
MKQLITTLFIFIFLQPSWASLYRNYQVEDGLSHNSVWAVMQDKQGFLWFGTVDGLNRFDG
NSFKIYKKLQGDSLSIGNNFIHCLKEDSHGHFLVGTKQGFYLFNRESETFSHVRLDNRSRGG
DDTSINYIMEDPDGNIWLGCYGQGIYVLGPDLQVRKHYINKGNPGDIASNHIWCMVQDYNG
VIWIGTDGGGLIRLDPKDERFTSIMHEKDLNLTDPTIYSLYCDMDNTIWVGTSISGLYRCNFR
TGKVTNIVYPHRKILNIKAITAYSNNELVMGSDAGLIKVDCIQETISFINEGPAFDNITDKSIFSI
AHDMEGGLWIGTYFGGVNYYSPYANKFAYYPGSSEEVSKSIISYFTEESSDKIWVGTKNEGL
LLFNPAKISFETTHLQIDYHDIQALMMDNDKLWISVYGKGVSMVDVHSNTLLKRYSNDVGG
PDLLTSNIVNVIFKSSKGQIFFGTPEGVDCLDAETKKINRLERTKGIPVKAIMEDYNGSIWFAA
HMHGLLHLSADGTWESFTHMPEDSTSLMSNNVNCIHQDARYRIWVGSEGEGMGLFNPKTK
KFEYILTENLGLPSNIIYAIQEDADGNIWVSTGGGLARIEPETRSICTFRYIEDLIKIRYNLNCAL
RGRDNHLYFGGTNGFIAFNPKDIQNNEYKPPICLTGFQISGNEVVPGIEGSPLKKSISMTQKIE
LESNQAAFSFDFVCLSYLSPAQNKYAYKLEGFDTDWHYVANGNNKAIYMNIPSGKYTFYV
KGTNNDGVWCDTPIKVTVIVKRHFWLSNMMLLVYAILAISAFTLLIRRYNKRLDSINQDKM
YKYKVEKEKEIYETKINFFTNMAHEIRTPLSLIVAPLENIISSGDGSQQTKSNLEIMKRNANRL
LELVNQLLDFRKIEEDMFRLCFSKQNISEIVRNIHKRYVQYAKLKDIDIRLVEPEKDIACVVD
KEAMEKVIGNLLSNAVKYANSLITINISTDNNLLTISVKDDGPGIKSEFIDKIFESFFQIENNAQ
RTGSGLGLALSKSLVTKHKGNIAASSDYGHGCTLTFTIPMDLPISISQLTEEYPEKEDISVQQT
ALSPVEGKLRIVLAEDNQELRSFLSNYLSDYLDVYEAQNGLEALQLVENENIDIIVSDILMPE
MDGLELCKALKSNPAYSHLPFILLSARTDTATKIEGLNTGADVYMEKPFSSEQLRAQINSIIN
NRNSIRENFIKSPLDYYKQKSAEPNGNTEFIEKLNIIILDNLTNEKFSIDNLSEMFLMSRSNLHK
KIKNIVGMTPNDYIKLIRLNQSAQLLATGKYKINEVCYLVGFNTPSYFSKCFYEHFGKLPKDF
IVIE
SEQ ID NO: 36-Mouse R. UCG13 GH5, truncated
GSAEINYNRSVPLEVKGNKIVKQGTDEMVVLRGVNVPSMDWGMAENLYESMTMVYDCW
GANLIRLPIHPKYWKDGSIWDGKNLTKEQYQKYIDDMVKAAQARGKYIILDCHRYVMPQQ
DDLDMLKELAVKYGNNSAVLFGLLNEPHDIKPTDIEKPTMEDQWEVWYNGGQIIVGGEEV
TAIGHQQLLNEIRALGANNICIAGGLSWAFDISGLADGYNGRENGYRLIDTAEGHGVMYDS
HAYPVKGTKSSWDTIIGPVRRVAPILIGEWGWDSSDNNISGGDCTSDIWMNQIMNWMDDTD
NQYDGIPLNWTAWNLHMKSSPRMISSWDYKTTAYNGTYIKNRLQSYGNLPETQDGVYSTD
FSTNDVFRGYKAPSGAASVSYSEANENIVVSHKPADWYATLNFPFDWDLNGIQTITMDISAD
TAETLNIGLYGSDMEEWTAPVKVDSTVKNITLSIDQLVRQGNQQTDGILNGAVSGIYIGSSTT
ETANNTKVVTMADEDNTAEYKINNYENGKVSVRKRTDAEDSASTVIVAFYDKNSVLTGIST
ANIRADEKGDEIIKAVNEPASYSCAEVFMWDSLNGMVPRCNPISNKVNITIDNIKIVKLAEPI
YTATEYPHTDIGAESYIDVDNTDFASQSTTKGAASTSYFTCENAEVVGADGENTQAKYITYD
RREGLYGGTVQFDLETVPSMDTKYFTISLKGSGTAQTINVNLGSEVSYNIALAEGDTDWHQ
YIFDISYGAQYPEDIAFVKLASNTKIESYFYADDFGFSKTKPERVIPNPEKTFIYDFATYNRNT
AKYEAVISTMPGSNDDEIRAEKVDGGLDFETQALEITYSRNGNIPSKTMVVYSPSDFFKGNS
NDDERTANRATLKADMEYMTDLVFYGKSTSDKNEKINVGVIDAANSMMTYTDTKEFTLTS
QWQQFRVPFDEFKVLDGGSELDCSRVRGFVFSSAENSGEGSFMIDNITHTSVADIEWAE
SEQ ID NO: 37-B salyersiae CE7
MRHRVILFICVLQTLFAYAVGAETHFMLTLNEQWKFSTGDSSAWATTEFDDNQWGTISSRQ
YWEEQGYDGYDGYGWYRQHFMISEDWKPIVTNAGGLYIRYEFADDVDEVFVNGVSVGRM
GEFPPEYKVIYGGMRKYKISPGLLRFGEENLIAIRVYDNGGAGGLKTENILLQSITPMDDLML
DIRCDDSDWVFENTETIDFRVRPKQPLAAGGEFNLVCSVTTDTYLPVDSFVYRVKGDFEQPV
SFVPPAPGFYRITLYGEQQGVKSDFLKFNMGYCPEQIISPVDVEPDFDQFWETTLKELSEVVP
DYRMTLLEEKSQGAKNIYRVEMYSLGNVRIEGYYAVPKQKGKFPSVISFLGYGSGGGFPRP
DNLPGFCEFILSTRGQGIQLPVNTYGKWIVHGLEDKSQYYYRGAFMDLVRGIDFLCSRPEVD
TEKIFAEGGSQGGAFTLAACALDRRICAAAPYIPFLSDFEDYFKIAPWPRSVFEEYLRSHEESS
WDEIYRLLSYFDSKNLAPRITCPIIMGVGLQDNICPPHINFSGYNQVKSPKRYYIYYDKEHTV
GKSWWTIRNNFFRSFCN
SEQ ID NO: 38-B salyersiae GH3 A
MKKLFKLFAFTCLAMSATAQNKTPIYLDETKPIEQRVEDALQRMTLEEKIKLCHAQSKFSSH
GVPRLGIPELWMTDGPHGIREEVLWDEWKGAAWTSDSCIAFPALTCLAATWDLDMSALYG
KSIGEEARFRGKDVLLGPGVNIYRTPLNGRNFEYMGEDPYLAAKMVVPYIKGVQQNGVAA
CVKHFALNNQEMYRGHINVEVSDRALHEIYLPAFKAAVLEGGTWSIMGAYNQYKGQHCCH
NQYLLNDILKKDWNFDGTVISDWGGVHDTYQSAYYGLDLEMGTWTDGLSWGKTNAYNN
YYMALPLLEKIKNGEIEENTVNDKVRRLLRMMFRTSMNTQKPWGSFGTEEHALAGRTIAEN
GIVLLKNENGLLPVDLSQIKKIAVIGENATKVMTLGGGSSSLKVKYEVSPLEGLKKRVGNAV
ELVYAPGYASPLTDKRDPRYIVLEGYRLPDAEKLTKEALEAAKNADIVLFFGGLNKNEHQD
SEGTDRLNYHLPYGQDELIAQLSKVNKNIAVILISGNAVAMPWIKEVPSVLEAWFSGTESGN
AIASVLVGDVNPSGKLPMTFAVRLEDYPAHTVGEYPGDSINVKYNEGIFVGYRWTDKHKIR
SLFPFGHGLSYTTFQYGKALLSSSEMNEKEILTVTIPIKNTGKVKGKEIVQLYIGDEKSSLERP
VKELKGFQKIELNPGEEKVVEFNITSNDLKFYDEAIQDWKAEQGKFNIFIGSSSTDIRAKTKF
NLK
SEQ ID NO: 39-B salyersiae GH3 B
MGVSVFAADDGGALYLDAGRPVEQRVKDLMSRMTLEEKVGQMCQWVGLEHMRTASQDL
TVDELSNNTARGFYPGITEEDVRQMTIDGKVGSFLHVLTVKEANQLQELAMKSRLKIPLIIGI
DAIHGNAQVVGTTAYPTSIGQASMFDVGLVEEICRQTALEMRATGSQWTFNPNVEVARDPR
WGRVGETFGEDPYLVSLLGVASVRGYQGDGFGKAENVLACAKHFIGGSQPINGINGSPTDI
SERTLREVFLPPFKATVDAGVYSFMTAHNELNGIPCHANPWLMEDILRKEWGFDGFIVSDW
MDIEHIHDLHRTAVDNKDAFYQSVDAGMDMHMHGPEFYEKVIELVKEGKLTEARIDESCR
KILAAKFRLGLFEKSFTDEKAAKSVLFNEKHQATALEAARKSIVLLTNDGILPLDEAKYKNV
FVTGMNADNQTILGDWALTQPDENVITVLEGLKLVSPDTKFSFVDLGWNIREMDKNKVEQ
AAKQAAKADLAIVAVGEYSLRTNWYDKTCGEDCDRSDINLAGLQQELVESILATGVPTVV
VLVNGRQLGVEWIAGHANALVEAWEPGSLGGQAIAEILYGKVNPSGKLPVTVPRHVGQIQ
MIYNHKPSMYFHPYAIGESTPLFYFGYGLSYTEYAYSDLTVSSAQMSGDGSVEVSVKVTNT
GTTDGEEIVQLYIRDLYSSATRPVKELKDFRRVPLRVGETKTVSFILPAGKLAFYDKKMDYT
VEPGDYEIMVGASSRDEDLMKRIVNVK
SEQ ID NO: 40-B salyersiae GH5_5
MEKKTKRIAFVLATMLCGWQMMLAQPVSPAPTPTRAANDVKAMFSDAYPEKFGKFQIDY
DDWNSDKFLTTKTIVTPFGAADEVLKIEGLSTGSLQHNAQIALGTCNLSDMEYLHMDVYSP
SENGIGEFSFYLVSGWSKTVSCNVWYNFDTKQEYDQWISIDIPMSTFKNGGLNLAEINVLRI
ARGKQGAPGTIVYVDNVYAYGKAVEPESDVKIVANGNANLTTDVPLISAPTPKVAAANVEN
FFSDHYGDGKFDYAQSDYGDQKTVKSLITINDTEDQVFKIDNIVNGSKANVSIGSPNLSGVD
MLHLDIFSPGNDQGIGEFDFALTDFGGNGNDAGIWLNITDKGWHGQWISIDIPLSKWTGAAN
MIRFRRGGKGSTGKLLYVDNVYAYKSESDDPKPVPDPTTVPVLTKDKSDVISIFCEQYEEPG
YQDEFGIVSAGNWGQNAKQKDEFVEIVAGNQTLKLTSWDLFPFKVHKNSDVMDLSQMDY
LHLSIYQNGALDENNKPVSVCIWINDKDNKVAQAPLLEVKQGEWTSVSFGMDYFKNKIDLS
RVYVIRLKVGGYPTQDIYVDNIFGYKGDPIRPGQVTEPYVDECDQKIQDSTPGTLPPMEQAY
LGVNLASASGGSNPGTFGHDYLYPKFEDLYYFKAKGIRLLRIPFRAPRLQHEVGGELDYDA
GNTSDIKALAAVVKEAERLGMWVMLDMHDYCERNIDGVLYEYGVAGRKVWDSAKNTWG
DWEAMDEVVLTKEHFADLWKKIATEFKDYTNIWGYDLMNEPKGININTLFDNYQAAIHAIR
EVDTKAQIVIEGKNYANAAGWEGSSDILKDLVDPVNKIVYQAHTYFDKNNTGTYKNSYDQ
EIGGNVEVYKQRIDPFIAWLEKNNKKGMLGEYGVPYNGHAQGDERYMDLIDDVFAYLKEK
QLTSTYWCGGSMYDAYTLTVQPAKDYCTEKSTMKVMEKYIKDFDTSIPSSLVETNADGNAI
VLYPNPVKDNLKITSESGIEQVIVFNMIGQKVSERNEKGTNIELNLEALGKGTYLVTVRLEDG
NVVNRKIVKM
SEQ ID NO: 41-B salyersiae GH88
MSCVLVCAGVLLLLSGLRETDVVGTKKQLSYCDTQIKKTLDAIEGSGLMPRCIDTDATDWY
KIDIYDWTSGFWPGILWYDYENTQNEEIRKAAIHYTESLVPLLDPEHPGDHDLGFQFYCSFG
NAYRLTKDDKYKQVLLKGADKLAGFYDPRVGTILSWPGMVTEMNWPHNTIMDNMMNLE
LLFWAAKNGGNREYYGMAVSHAKVTKENQFRPDGSCYHVAVYDTIDGRFLKGVTNQGYS
DSSLWARGQAWAIYGYTLVYRETGDKEYLRFAEKITDIYLKRLPEDYVPYWDFDDPAIPDA
PRDASAAAIVASGLLELVQLEDNTEKAEEYRDAAVNMLLSLSSDAYQSGIKKPSFLLHCTGN
LPGGYEIDASINY ADYYYIEALTRYKKMQAGRDIVEKYPQATQKQVTIAM
SEQ ID NO: 42-B salyersiae GH92_GH5
MKSHPLLILLIIIPTCLFAGNPDKVSLVDMFMGVKNSSNCVIGPQLPHGSVNPAPQTPNGGHN
GYDENDVIRGFGQLHVSGIGWGRYGQVFISPQVGFKPGETEHDSPKSDEVATPYYYKVNLD
RYKIKTEITPTHHSVYYRFTYPKSGNKNILLDMKHNIPQHIVPIVKGTFLGGNIEYDKASGLLT
GWGEYAGGFGSAAPYKVFFAMRPDVKLKEVKVTDKGTKALYARLSLPEEAETVHLGIGVS
LRSVENACKYLEQEIGARSFDEVKRVAKSAWEDVFATIDVKGGTQEEQRLFYTAMYHSFV
MPRDRTGDNPRWTSGQPHLDDHFCVWDTWRTKYPLMMLVNESFVAKTVNSFIDRFAHDG
ECTPTFTSSLEWEMKQGGDDVDNIIADAFVKNLKGFDRQKAYELVKWNAFHARDSLYLKK
GWIPETGARMSCSYTMEYAYNDDCGARIARIMKDDETADYLENRSQQWVNLFNPNLESHG
FNGFVGPRKENGEWIGIDPALRYGPWVEYFYEGNSWVYTLFAPHQFSRLIRLCGGKEAMAD
RLTYGFEKELIELDNEPGFLSPFIFSHCDRPGQTAKYVDFIRKNHFSRATGYPENEDSGAMGA
WYIFTSIGFFPNAGQDFYYLLPPAFSEVTLTMENGKKIDIKTVKSTPEVNYIESVSLNGKLLDR
TWIRHAEIAEGATIVYHLTDKPGQWSISPFEASRREPQPFGVNLAGAEFFHKKMEGVGRFNK
DYHYPTTDELDYWKSKGLTLIRLPFKWERIQRKLYGELNREEMDYIKFLLAEADKRDMQILI
DMHNYGRRKDDGKDRIIGDSLSIDHFASAWGSISRELKDCKGLYGYGLINEPHDMLASTPW
VGIAQAAIDSIRKNDAKNAIVVGGNHWSSAERWKLVSDDLKNLRDPSRNLIFEAHCYFDED
GSGIYRRSYEEEKAHPYIGVERMRPFVEWLKENDFRGLVGEYGVPADDERWLECLDNFLAY
LSAEGVNGTYWAAGARWNRYILSVHPENDYRKDKPQMKVLMKYLRTQ
SEQ ID NO: 43-B salyersiae HTCS
MKHTILVLLGLALSFFPARAYHFRSYQVEDGLSHNSVWAVMQDSKGFMWFGTNDGLNRF
DGKKIKVYRKIQGDSLSIGNNFIHCLKEDSRGRFLIGTKQGLYLFDDKLEKFRHIDLDKNIKD
DVSINAIMEDPSGNIWLACHGYGLYVLTPELTTKKHYLSGSDPYSLPSNYIWSIVQDYYGNI
WLGTVGKGLVHFDPKEEKFTQMTQAKELGIDDPVIYSLYCDIDNNIWIGTATSGLIRYTPRS
QKATHYINHVFNIKSIIEYSDHELIMGSDKGLVKFDRTLESFDLINDDTSFDNMTDKSIFSIAR
DKEGSFWIGTYFGGVNYYSPAINRFQYCYNSPHNSSKKNIISGFAENENGDIWIGTHNDGLY
LFNPKSLSFKKPYDIGYHDVQSILSDQDKLYASLYGKGIHILNIKNGQVSASANDIGINHTINS
IAKTSKGQILFTSEGGVISMDASGTLKTLDYLTNTPVKDIAEDYDGSIWFATHSKGLIRLTSD
NRWEVFVNNPDNPKSLPGNNVNCVFQDSKFHIWAGTEGEGLVRFNAKEQNFEPILNDQSGL
PSNIIYSILDDSDGNLWVSTGGGLVKISSDLKNIKTFAYIGDIQRIQYNLNCALRASDNRLYFG
GTNGFITFNPKEITDNPNKPVVMVTGFQIASKEITLSESSPLKETISATKEITLRHDQSTFSFDF
VALSYLSPEQNRYAYILEGFDKEWHYTSDNKAMYMNIPPGTYVFRVKGTNNDGVWSDETA
DITVKIKPPFWLSNLMIGLYIVLAIGIILYFIRRYHRFIERKNQEKIFKYQTAKEKEMYESKINF
FTNIAHEIRTPLSLIAAPLEKIILSGDGNEQTRNNLGMIERNANRLLELINQLLDFRKIEEDMFH
FKFKRQNVVKIVEKVYKQYYQTAKFNKLEISLEAEKNDIECNVDSEAIYKIVSNLIANAIKYA
KSQILITVKERSGNLEIKIKDDGTGIEKQYMEKIFEPFFQIQDKNNAVRTGSGLGLSLSQSLAM
KHNGKISIESEYGKNCNFTLTIPIADGTEEEVQETEAAIPEKSEMPEQSVVEAGTRIIIVEDNTD
MRTFLCESLNDNYTVFEAENGVQALEMVEKENIDIIISDIMMPEMDGLELCNRLKSDPAYSH
LPLVLLSAKTDTSTKIEGLNQGADVYMEKPFSIEQLKAQISSIIENRNNLRKNFIKSPLQYFKQ
NTENNESADFVKKLNTIILENMSDEDFSIDSLSSQFAISRSNLHKKIKNITGMTPNDYIKLIRLN
ESARMLSTGKYKINEVCFLVGFNTPSYFSKCFFEQFKKLPKDFIQITNE
SEQ ID NO: 44-B salyersiae NZ KB905466
MKKQFSTLIALLIVGAAPLLGQETDPLNDPTNIDADLYLHAGFSQDSIRPDYSHTYYDNINH
KLVKGEDGIYSITVPLKKEQIVNKNMEVGIYTYAYSVIYGGKVNGSGNDAVKGSVGPVIAD
EPRLFELAEDRDVTFYAKKLNTGTADAPWYRTMFICDAQPLYLDGTELPLPGEDGVTRYVV
DRGETSRRWEYKLSPIGRWSKTQDFMEDVIPAKWKSNEAYAFLPNGGWWLGGRFLLAYD
YKKLSLEVGKLVDELQTPLFTVNGESIPENLGIVDELLLNGSVITFLKGYYANGGKDSYDPA
FNTSIATVKLCWQIDELPAASFPLTNGEVVRDDNYNKTTEWTVSEADLFEGTTLPAGIHTLK
VWYESEYLGDVLTSEVQSTSFEIEEIVVIPLENKGTAVDLILEGDWNPETFRTIIEEQAVRITTI
DLTGVAGLTELPEMEGLNPNCLVYVNPDVVIAEGVDNVVVFDNEEGRAANILLTEGSDENN
VRLFTADRISYSHNFTADVWSTICLPFSADKGDVTVEEFTGADGEKVIFTGTSAIEANVPYLA
KTSNSEVKTFTATDVQMSVTAEPAPVVPENGYAFHAGYRAVEGDAVVGLHLMNDVGTAF
VKVADGNPEAAGVSAFHAYMQATVDELLTIVHGDDNPTGLGSTEDTGRLTIISHNGSVEIKT
GKAQMIGLYALDGRLVKMVELSQGSNFVNGLDKGIYIMDCQKVVVK
SEQ ID NO: 45-B salyersiae putative PL
MKKIITIAFLSFLYFVYGYASNHSMHPLKQIDYVLKQVKAQQEPYYSAYQQLIHDADSILKV
SHHALVDFAVPGFYDKPEEHRANSLALQRDAYAAYCSALAYTLSGQQEYGEKACYFLNAW
ASTNEKYSEHDGVLVMTYSGSAFLMAAELMADDPLWSNKEKKDFRKWVKRIYQHAANTI
RVHQNNWADWGRFGSLLAASFLNEKKEVAENVRLIKSDLFHKIATDGSMPEETRRGGNGI
WYTYFSLAPMTGACWLVYNLTGENLFALEQDGTSIKKALDYMAYYNKHPKEWKWDKNP
NTGKNEVWPENLLEAMANLYNDNSYVEYVKGKRPIIYRNHHFCWTFPTLMPTSFENYQ
SEQ ID NO: 46-B salyersiae SusC
MISKDENIKRRIIGVLFFLCALSPALWAQSRIIKGEVLDPNGEPLIGVGVMIKNTTAGTITDVD
GRYSIQVPDNNAVLSFSYVGYKRKEVKVGSQSVINISLEEESVLMDQVVIVGYGSQKKVNLT
GAVAAISVDESLAGRSVANVSSALQGLMPGLSVSQSSGMAGNNSAKLLIRGLGTINSADPLI
VVDDMPDADINRLNMNDIESITVLKDATASSVYGSRAANGVILVKTKSGKGLEKTQITFSGS
YGWEKPTNTYDFISNYPRALTLQQISSSTNPGKNGENQNFKDGTIDQWLALGMIDDKRYPN
TDWWDYIMRTGSIQNYNVSATGGSEKSNFYASVGYMKQEGLQINNDYDRYNARFNFDYK
VMKNVNTGFRFDGNWSNFTYALDNGFTSDSNLDMQSAIAGIYPYDPVLDVYGGVMAYGE
DPQAFNPLSFFTNQLKKKDRQELNASFYLDWEPVKGLVARVDYGLKYYNQFYKEADIPNR
SYNFQTNSYGIREYVTENAGVTNQTSTGYKTLLNARLNYHTVFATHHDLNAMFVYSEEYW
HDRYQMSYRQDRIHPSLSEIDAALSGTQSTSGNSSAEGLRSYIGRINYSAYGKYLLELNFRVD
GSSKFQPGHQYGFFPSAALGWRFSEESFVKPYIGKWLASGKLRASYGKLGNNSGIGRYQQQ
EVLYQNNYMLDGSIAKGFVYSKMLNPDLTWESTGVFNLGLDLMFFDGKLAAEFDYYDRLT
TGMLQKSQMSILLTGAYEAPMANLGTLRNRGFEANLTWRDRIADFTYSANFNISYNRTNLE
KWGEFLDKGYVYIDMPYHFVYSQPDRGLAQTWTDSYNATPQGVAPGDVIRLDTNGDGRID
GNDKVAYTNFQRDMPTTNFALNLQMGWKGIDVSLLFQGSAGRKDFWNNKYTEINLPDKR
YTSNWDQWNKPWSWENRGGEWPRLGGLVTNKTETDFWLQNMTYLRMKNLMIGYTFPKK
WTRKCFIENLRIYGTAENLLTITGYKGLDPEKAANSQDLYPITKSYSIGVNLSF
SEQ ID NO: 47-B salyersiae SusD
MKRVYIKYIGLIAGMMMLFSSCADLLNQEPTVDLPATNYWKTESDAESALNGLVSDIRWLF
NRDYYLDGMGEFVRVRGNSFLSDKGRDGRAYRGLWEINPVGYGGGWSEMYRYCYGGINR
VNYVIDNVEKMIANASSEKTIKNLEGIIGECKLMRALVYFRLIMMWGDVPYIDWRVYDNSE
VENLPRTPLAEVKDHILDDLLDAFKKLPEKATVEGRFSQPAALALRGKVLLYWASWNHYG
WPELDTFTPSEEEARKAYKAAAEDFRTVIDDYGLTLFRNGEPGECDEPGKADKLPNYYDLF
LPTANGDAEFVLAFNHGGTNTGQGDQLMRDLAGRSVENSQCWVSPRFEIADKYQSTITGDF
CVPLVKLNPSSVPDARTRPNSAVNPESYKDRDYRMKASIMWDYEICQGLMSKKVTGWVPFI
YKMWGSEVVINGETYMSYNTDGTNSGYVFRKFVRNYPGEERADGDFNWPVIRLADVFLM
YAEADNAVNGPQPYAIELVNRVRHRGNLPVLASSKTSTPEAFFEAIKQERIVELLGEGQRAF
DTRRWREIETVWCEPGGRGVKMYDTYGAQVAEFYVNQNNLAYERCYIFQIPESERNRNPN
LTQNKPYR

1. A polypeptide comprising a xanthan lyase, wherein the xanthan lyase comprises an amino acid sequence having at least 70% sequence identity to SEQ ID NOs: 1-4.

2. (canceled)

3. The polypeptide of claim 1, wherein the polypeptide does not comprise SEQ ID NO: 5.

4. A polynucleotide comprising a nucleic acid sequence encoding the polypeptide of claim 1.

5. (canceled)

6. A host bacterial cell comprising the polynucleotide of claim 4.

7. (canceled)

8. A composition comprising the polypeptide of claim 1.

9. The composition of claim 8, wherein the composition is a cleaning composition, a well treatment composition, or a wellbore servicing composition.

10-11. (canceled)

12. A method of cleaning comprising contacting an object or a surface with the polypeptide claim 1, or a composition comprising thereof.

13-14. (canceled)

15. A method of degrading xanthan gums or xanthan gum oligosaccharides comprising:
contacting xanthan gum, xanthan gum oligosaccharides, or a composition comprising thereof with the polypeptide of claim 1, or a composition comprising thereof.

16. A genetically modified bacterium comprising a polynucleotide encoding at least one heterologous xanthan lyase comprising an amino acid sequence having at least 70% sequence identity to any of SEQ ID NOs: 1-5.

17-18. (canceled)

19. The genetically modified bacterium of claim 16, wherein the genetically modified bacterium further comprises one or more nucleic acids encoding one or more glycoside hydrolase, one or more carbohydrate uptake protein, one or more carbohydrate esterase, one or more polysaccharide lyase, or a combination thereof.

20. The genetically modified bacterium of claim 19, wherein the one or more glycoside hydrolase is, or is derived from, a glycoside hydrolase family 5 enzyme, a glycoside hydrolase family 88 enzyme, a glycoside hydrolase family 94 enzyme, a glycoside hydrolase family 38 enzyme, a glycoside hydrolase family 92 enzyme, a glycoside hydrolase family 3 enzyme, or a combination thereof.

21. The genetically modified bacterium of claim 20, wherein the glycoside hydrolase family 5 enzyme is or is derived from Ruminococcaceae UCG13.

22. The genetically modified bacterium of claim 20, wherein the glycoside hydrolase family 5 enzyme comprises an amino acid sequence having at least 70% sequence identity to SEQ ID NO: 7, 8, or 36.

23. The genetically modified bacterium of claim 20, wherein:
the glycoside hydrolase family 88 enzyme comprises an amino acid sequence having at least 70% identity to SEQ ID NO: 13, 21, or 41;
the glycoside hydrolase family 94 enzyme comprises an amino acid sequence having at least 70% identity to SEQ ID NO: 10;
the glycoside hydrolase family 38 enzyme independently comprises an amino acid sequence having at least 70% identity to SEQ ID NO: 11 or 12;
the glycoside hydrolase family 92 enzyme independently comprises an amino acid sequence having at least 70% identity to SEQ ID NO: 22 or 42; and/or the glycoside hydrolase family 3 enzyme independently comprises an amino acid sequence having at least 70% identity to SEQ ID NO: 18, 38 or 39.

24-27. (canceled)

28. The genetically modified bacterium of claim 19, wherein:
the one or more polysaccharide lyase family protein independently comprises an amino acid sequence having at least 70% identity to SEQ ID NO: 9, 19, or 45;
the one or more carbohydrate esterase independently comprises an amino acid sequence having at least 70% identity to SEQ ID NO: 14 or 15; and/or the one or more carbohydrate uptake protein independently comprises an amino acid sequence having at least 70% identity to SEQ ID NO: 16, 17, 46, or 47.

29-30. (canceled)

31. The genetically modified bacterium of claim 16, wherein the bacterium is from the genus Bacteroides, Parabacteroides, Alistipes, Prevotella, Clostridium, Faecalibacterium, Eubacterium, Ruminococcus, Peptococcus, Peptostreptococcus, Bifidobacteria, Escherichia, or Lactobacillus.

32. The genetically modified bacterium of claim 16, wherein the bacterium is a gram-positive gut commensal bacteria.

33-36. (canceled)

37. A composition comprising the genetically modified bacterium of claim 16.

38. A method for treating a disease or disorder in a subject comprising administering to the subject a genetically modified bacterium of claim 16, or a composition comprising thereof.

39. The method of claim 38, wherein the disease or disorder comprises a gastrointestinal disease or disorder.

40. (canceled)
